(19) United States
(12) Patent Application Publication
(10) Pub. No.: US 2026/0146014 A1
(43) Pub. Date: May 28, 2026

(54) METHODS FOR PRODUCING PHENOLIC COMPOUNDS INCLUDING SYRINGOL FROM BIOMASS AND USING SAME FOR 3D PRINTING

(71) Applicant: Align Technology, Inc., San Jose, CA (US)

(72) Inventors: Jian Chang, San Francisco, CA (US); Ming Ren, Belmont, CA (US); Daniel Yilin Jiang, Fremont, CA (US); Lance Robert Pickens, Campbell, CA (US); Xiance Wang, Santa Clara, CA (US); Jessica Kalay Su, San Jose, CA (US)

(21) Appl. No.: 19/090,026

(22) Filed: Mar. 25, 2025

Related U.S. Application Data
(60) Provisional application No. 63/570,115, filed on Mar. 26, 2024.
(60) Provisional application No. 63/719,823, filed on Nov. 13, 2024.
(60) Provisional application No. 63/723,749, filed on Nov. 22, 2024.

(51) Int. Cl. C07C 41/01; B33Y 70/00; C08F 20/30

(52) U.S. Cl. CPC C07C 41/01; B33Y 70/00; C08F 20/30

(57) ABSTRACT

Methods for the production of syringol from biomass are provided. Reactive diluents derived from syringol and their use in 3D printing are also provided.

### PRIORITY CLAIM AND CROSS-REFERENCE

This application claims the benefit of U.S. Provisional Patent Application No. 63/570,115, filed Mar. 26, 2024, U.S. Provisional Patent Application No. 63/719,823, filed Nov. 13, 2024, and U.S. Provisional Patent Application No. 63/723,749, filed Nov. 22, 2024, each of which is herein incorporated by reference in its entirety.

### BACKGROUND

Resins for additive manufacturing are complex formulations comprising photoinitiators, monomers, and oligomers, designed to achieve polymeric materials with tailored physical, chemical, and thermomechanical properties (e.g., elongation, stress relaxation, modulus, durability, and toughness) for applications such as dental aligners, hearing aids, and medical devices. A key challenge in this field is balancing resin components to ensure good processability while maintaining performance. Syringol-based monomers, such as syringyl (meth)acrylates, serve as reactive diluents in 3D printable resins, imparting low vapor pressure to reduce viscosity and improve processibility of the resins while preserving the desired physical properties of the resulting polymeric material.

Syringol (2,6-dimethoxyphenol), obtained from biomass-such as through lignin depolymerization-serves as a precursor to syringyl (meth)acrylate. Consequently, syringyl (meth)acrylate and other syringol-based monomers present a potentially sustainable and cost-effective approach to tailoring resin processability. However, commercially available syringol is primarily derived from tree charcoal oils, which are neither renewable nor sustainable. Therefore, there remains a need to produce syringol from renewable resources, particularly from non-food biomass feedstocks.

### BRIEF SUMMARY

The present disclosure provides economically feasible processes for producing syringol from renewal and sustainable feedstocks derived from biomass.

In one aspect, methods for the production of syringol from a biomass are provided.

In some embodiments, the method includes introducing a composition comprising the biomass and a solvent into a reactor, feeding an oxygen source into the reactor, heating the composition at a temperature below 200° C. while continuously feeding the oxygen source into the reactor to depolymerize the lignin in the biomass, thereby yielding a depolymerized lignin composition comprising syringol, and obtaining syringol from the depolymerized lignin composition. In some embodiments, the oxygen source is fed into the reactor at a rate of 0.2 L/min to 5 L/min. In some embodiments, the depolymerized lignin composition further comprises syringaldehyde, syringic acid, vanillin, and vanillic acid.

In some embodiments, the method includes introducing a composition comprising the biomass and a solvent into a reactor, feeding a first amount of an oxygen source into the reactor, heating the composition at a temperature below 200° C. until oxygen the first amount of the oxygen source is depleted to depolymerize lignin in the biomass, thereby yielding a depolymerized lignin composition comprising syringaldehyde and syringic acid, and converting syringaldehyde and syringic acid into syringol. In some embodiments, the method further comprises steps of: (a) feeding a second amount of the oxygen source into the reactor, (b) heating the composition at a temperature blow 200° C. until the second amount of oxygen in the oxygen source is depleted, and (c) optionally repeating steps (a) and (b) prior to converting syringaldehyde and syringic acid into syringol. In some embodiments, steps (a) and (b) are repeated one or more times. In some embodiments, steps (a) and (b) are repeated two times.

In some embodiments, the solvent comprises water, methanol, ethanol, acetone, acetonitrile, methylene chloride (MEC), perchloroethylene (PCE), trichloroethylene (TCE), polyethylene glycol (PEG-200 or PEG-400), sulfolane, γ-valerolactone (GVL), N-methylpyrrolidone (NMP), or a mixture thereof. In some embodiments, the solvent comprises supercritical water, supercritical methanol, or supercritical ethanol. In some embodiments, the oxygen source comprises an oxygen gas or air. In some embodiments, the oxygen gas is balanced with a nitrogen gas. In some embodiments, the oxygen source comprises at least 2 vol % of oxygen. In some embodiments, the depolymerization of lignin is carried out at a pressure below 600 psi. In some embodiments, the depolymerization of lignin is carried out at 190° C.

In some embodiments, the method includes treating the biomass with an enzyme composition to separate a lignin fraction from cellulose and hemicellulose fractions, subjecting the lignin fraction to a biological depolymerization process to depolymerize lignin in the biomass, thereby yielding a depolymerized lignin composition comprising syringaldehyde and syringic acid, and producing syringol from the depolymerized lignin composition. In some embodiments, the enzyme composition comprises one or more enzymes selected from laccases, cellobiohydrolases, endoglucanases, β-D-glucosidases, xylanases, arabinofuranosidases, acetyl xylan esterases, glucuronidases, mannanases, galactanases, arabinases, lignin peroxidases, manganese-dependent peroxidases, hybrid peroxidases, and ferulic acid esterases. In some embodiments, the enzyme composition further comprises a mediator. In some embodiments, the mediator comprises catechol, guaiacol, violuric acid, or 1-hydroxy-benzotriazole (HBT). In some embodiments, a weight ratio of the one or more enzymes to the biomass ranges from 1:10 to 1:100. In some embodiments, treating the biomass with the enzyme composition is conducted at a temperature ranging from 25° C. to 75° C. In some embodiments, the biological depolymerization process is carried out by contacting the lignin fraction with a biocatalyst. In some embodiments, the biocatalyst comprises a lignolytic microbe composed of fungi, bacteria, and achaca or an engineered microbe. In some embodiments, the bacteria comprises E. coli, bacillus, Streptomyces, or Rhodococcus species. In some embodiments, the method further comprises removing non-lignin cellulose and hemicellulose components from the biomass using a solvent. In some embodiments, the solvent comprises water, ethanol, isopropanol, acetone, and mixtures thereof.

In some embodiments, the method includes subjecting the biomass to a catalytic fractionation process to depolymerize lignin in the biomass, thereby yielding a depolymerized lignin composition comprising syringaldehyde and syringic acid, and producing syringol from the depolymerized lignin composition. In some embodiments, the catalytic fractionation process is carried out by contacting the pretreated biomass with a lignin depolymerization catalyst at a temperature below 200° C. In some embodiments, the catalytic fractionation process is carried out at 190° C. In some embodiments, the lignin depolymerization catalyst comprises a transition metal oxide supported by a support material. In some embodiments, the transition metal oxide comprises ZnO, CoO, Co₃O₄, CuO, MnO, or Fc₂O₃. In some embodiments, the support material comprises silica, alumina, titania, zirconia, magnesium oxide, silica-alumina, carbon black, zeolites, and mixtures thereof. In some embodiments, the lignin depolymerization catalyst has a Brunauer-Emmett-Teller (BET) surface area ranging from 8 m²/g to 250 m²/g. In some embodiments, the oxidation agent comprises oxygen gas, air, or oxygen-enriched air. In some embodiments, the lignin depolymerization catalyst includes a transition metal-based catalyst and a solvent. In some embodiments, the transition metal-based catalyst comprises zinc sulfate (ZnSO₄), zinc acetylacetonate (Zn(C₅H₇O₂)₂), zinc acetate (Zn(CH₃COO)₂), cobalt chloride (CoCl₂), cobalt acetylacetonate (Co(C₅H₇O₂)₂), cobalt acetate (Co(CH₃COO)₂), copper sulfate (CuSO₄), copper acetate (Cu(CH₃COO)₂), manganese sulfate (MnSO₄), manganese acetylacetonate (Mn(C₅H₇O₂)2), manganese acetate (Mn(CH₃COO)₂), Iron acetylacetonate (Fe(C₅H₇O₂)₃), iron chloride (FeCl₃), ferric citrate, ferric acetate (Fe(CH₃COO)₃), or iron phthalocyanine (FePc). In some embodiments, the solvent comprises water, methanol, ethanol, acetone, acetonitrile, anisole, cyclohexanone, methylene chloride (MEC), perchloroethylene (PCE), trichloroethylene (TCE), polyethylene glycol (PEG-200 or PEG-400), sulfolane, γ-valerolactone (GVL), N-methylpyrrolidone (NMP), or a mixture thereof. In some embodiments, the solvent comprises supercritical water, supercritical methanol, or supercritical ethanol.

In some embodiments, producing syringol from the depolymerized lignin composition comprises treating the depolymerized lignin composition with an oxidation agent, wherein the oxidation agent converts syringaldehyde to syringic acid; and performing a decarboxylation reaction to convert syringic acid to syringol, thereby affording a syringol-containing crude product. In some embodiments, the oxidation agent comprises an oxygen gas, air, or oxygen-enriched air. In some embodiments, the oxidation agent further comprises a carrier gas selected from nitrogen, argon, and helium. In some embodiments, treating the depolymerized lignin composition with an oxidation agent is carried out in the presence of bis(methoxypropyl) ether.

In some embodiments, performing the decarboxylation reaction comprises contacting the depolymerized lignin composition with a decarboxylation catalyst, wherein the decarboxylation catalyst converts syringic acid to syringol. In some embodiments, the decarboxylation catalyst comprises CuCl, Cu(NO₃)₂, Cu₂O, CuO, Cu, CuSO₄, Cu(OAc)₂, or CuCl₂. In some embodiments, the decarboxylation of syringic acid is carried out at a temperature of 120° C. or more, 150° C. or more, 180° C. or more, 200° C. or more, or 250° C. or more. In some embodiments, the method further comprises purifying the syringol-containing crude product to produce syringol.

In some embodiments, the method includes pyrolyzing the biomass under an inert atmosphere to decompose lignin in the biomass, thereby affording a syringol-containing crude product, and purifying the syringol-containing crude product to afford syringol. In some embodiments, pyrolyzing the biomass is carried out at a temperature ranging from 500° C. to 600° C. In some embodiments, purifying the syringol-containing crude product comprises performing a selective solvent extraction using a basic aqueous solution comprising a base and a polar solvent, thereby affording a syringol-enriched bio-oil. In some embodiments, the base is KOH or NaOH. In some embodiments, the polar solvent comprises methyl tert-butyl ether (MTBE). In some embodiments, purifying the syringol-containing crude product further comprises distilling the syringol-enriched bio-oil to afford syringol. In some embodiments, purifying the syringol-containing crude product further produces vanillin or guaiacol.

In some embodiments, the method further comprises pretreating the biomass by a mechanical means. In some embodiments, pretreating the biomass comprises milling or grinding the biomass to afford biomass particles. In some embodiments, an average size of the biomass particles is from 70 μm to 1,200 μm. In some embodiments, the biomass comprises nutshells. In some embodiments, the nutshells are selected from shells from walnuts, peanuts, pines, almonds, and cashews. In some embodiments, the nutshells are walnut shells. In some embodiments, the biomass comprises a woody part from a hardwood. In some embodiments, the hardwood is selected from oak, walnut, beech, maple, ash, and poplar. In some embodiments, the biomass comprises corn stover, wheat straw, rice stalk, barley straw, sugarcane bagasse, peanut shell, or soybean hull. In some embodiments, the biomass comprises a bioengineered source obtained from microbes.

In some embodiments, the method includes extracting a tannic acid extract from the biomass, hydrolyzing tannic acid in the tannic acid extract to provide gallic acid, decarboxylating gallic acid to provide pyrogallol, forming 1,2,3-trimethoxybenzene by reacting pyrogallol with a methylation reagent, preparing a first solution comprising 1,2,3-trimethoxybenzene and a solvent, adding a second solution comprising a demethylation reagent and the solvent to the first solution to form a reaction mixture, heating the reaction mixture at a reaction temperature above 0° C. for a period of time sufficient to convert the 1,2,3-trimethoxybenzene to syringol, and isolating the syringol. In some embodiments, the solvent comprises dimethylformamide, dichloromethane, chloroform, heptane, tetrahydrofuran, 2-methyltetrahydrofuran, diglyme, 1,4-dioxane, chlorobenzene, benzene, anisole, acetonitrile, 2-dichloroethane, or a mixture thereof. In some embodiments, the solvent is dichloromethane or heptane. In some embodiments, the demethylation reagent comprises ZnCl₂, LiCl, AlCl₃, BCl₃, FeCl₃, or a combination thereof. In some embodiments, the demethylation reagent is AlCl₃. In some embodiments, the demethylation reagent is BCl₃. In some embodiments, the demethylation reagent is AlCl₃ and BCl₃. In some embodiments, the demethylation reagent comprises a trifluoroboron ether complex. In some embodiments, the demethylation reagent is BF₃·OEt₂. In some embodiments, the demethylation reagent further comprises a quaternary ammonium salt. In some embodiments, the quaternary ammonium salt comprises tetra-n-butylammonium bromide or tetra-n-butylammonium iodide. In some embodiments, the demethylation reagent comprises a mixture of a BF₃·OEt₂ and tetra-n-butylammonium bromide. In some embodiments, the demethylation reagent comprises a Grignard reagent having the structure of RMgX, wherein R is C₁-C₆ alkyl or C₃-C₆ cycloalkyl, and X is halogen. In some embodiments, R is methyl, ethyl, isopropyl, butyl, tert-butyl, sec-butyl, and cyclopropyl. In some embodiments, X is Br, Cl, or I. In some embodiments, the demethylation reagent comprises MeMgBr, MeMgCl, EtMgBr, EtMgCl, iPrMgCl, iPrMgBr, or mixtures thereof. In some embodiments, the demethylation reagent is MeMgBr or MeMgCl. In some embodiments, the demethylation reagent comprises halosilane. In some embodiments, the demethylation reagent is iodotrimethylsilane (Me₃Sil) or chlorotrimethyl (Me₃SiCl). In some embodiments, a molar ratio of the demethylation reagent to 1,2,3-trimethoxybenzene ranges from 0.1:1.0 to 5.0:1.0, 0.1:1.0 to 4.0:1.0, 0.1:1.0 to 3.0:1.0, 0.1:1.0 to 2.0:1.0, or 1.0:1.0 to 2.0:1.0. In some embodiments, a molar ratio of the demethylation reagent to 1,2,3-trimethoxybenzene ranges from 1.1:1.0 to 1.5:1.0. In some embodiments, a molar ratio of the demethylation reagent to 1,2,3-trimethoxybenzene is about 1.5:1.0. In some embodiments, a concentration of the demethylation reagent is ranges from 1M to 5M. In some embodiments, a concentration of the demethylation reagent ranges from 1M to 3M. In some embodiments, a concentration of the demethylation reagent is about 3M. In some embodiments, the reaction temperature ranges from 40° C. to 50° C., from 5° C. to 20° C., from 10° C. to 20° C., from 10° C. to 30° C., from 10° C. to 40° C., or from 10° C. to 50° C. In some embodiments, the reaction temperature is about 45° C. In some embodiments, the second solution is added to the first solution over a period of time ranging from 10 to 20 minutes, from 10 to 30 minutes, from 10 to 40 minutes, from 10 to 50 minutes, or from 10 to 60 minutes. In some embodiments, the second solution is added to the first solution by a dropwise manner or in portions. In some embodiments, the second solution is added to the first solution in three or more portions over 10 to 20 minutes, from 10 to 30 minutes, from 10 to 40 minutes, from 10 to 50 minutes, or from 10 to 60 minutes. In some embodiments, the second solution is added to the first solution in ten or more portions over 10 to 20 minutes, from 10 to 30 minutes, from 10 to 40 minutes, from 10 to 50 minutes, or from 10 to 60 minutes. In some embodiments, the syringol is obtain with at least 92% purity. In some embodiments, the syringol is obtained with over 90% yield. In some embodiments, the method further comprises pretreating the biomass by a mechanical means. In some embodiments, pretreating the biomass comprises milling or grinding the biomass to afford biomass particles. In some embodiments, an average size of the biomass particles is from 70 μm to 1,200 μm. In some embodiments, the biomass comprises gallnuts, tree barks, leaves, husks, and pods from plants. In some embodiments, the biomass comprises oak gallnuts, tara tree barks, oak bark, sumac leaves, tea leaves, pomegranate husks, or tara pods. In some embodiments, extracting the tannic acid extract from the biomass comprises contacting the biomass with a solvent and isolating the tannic acid extract. In some embodiments, the solvent is water, acetone, methyl ethyl ketone, ethyl acetate, methyl acetate, ethanol, isopropanol, 1,4-dioxane, hexane, tetrahydrofuran, or a mixture thereof. In some embodiments, the extracting the tannic acid extract is carried out at 20° C. to 60° C. In some embodiments, hydrolyzing tannic acid is carried out using hot water or enzyme.

In still another aspect, a method for synthesizing syringol from 4-hydroxybenzoic acid is provided. In some embodiments, the method includes halogenating 4-hydroxybenzoic acid to form 3,5-dihalogenated 4-hydroxybenzoic acid, reacting the 3,5-dihalogenated 4-hydroxybenzoic acid with an alkoxide to form 3,5-dimethoxy-4-hydroxybenzoic acid, and decarboxylating the 3,5-dimethoxy-4-hydroxybenzoic acid to form syringol. In some embodiments, the 3,5-dihalogenated 4-hydroxybenzoic acid is 3,5-dibromo 4-hydroxybenzoic acid, 3,5-dichloro 4-hydroxybenzoic acid, 3,5-difluoro 4-hydroxybenzoic acid, or 3,5-diiodo 4-hydroxybenzoic acid. In some embodiments, the 3,5-dihalogenated 4-hydroxybenzoic acid is 3,5-dibromo 4-hydroxybenzoic acid. In some embodiments, the alkoxide is NaOCH₃, KOCH₃, or LiOCH₃. In some embodiments, the 3,5-dihalogenated 4-hydroxybenzoic acid is reacted with a metal halogen to form 2-hydroxyl 4,6-dimethoxy benzoic acid. In some embodiments, the metal halogen is CuBr, Cul, CuCl, or CuF. In some embodiments, the 3,5-dibromo 4-hydroxybenzoic acid is reacted with NaOCH₃ and CuBr to form 2-hydroxy 5,3-dimethoxy benzoic acid. In some embodiments, the 3,5-dichloro 4-hydroxybenzoic acid is reacted with NaOCH₃ and CuBr to form 2-hydroxy 3,5-dimethoxy benzoic acid. In some embodiments, the 3,5-dibromo 4-hydroxybenzoic acid is reacted with a metal halogen at a temperature from 80° C. to 120° C. to form 2-hydroxy 3,5-dimethoxy benzoic acid. In some embodiments, the 3,5-dichloro 4-hydroxybenzoic acid is reacted with a metal halogen at a temperature from 80° C. to 120° C. to form 2-hydroxy 3,5-dimethoxy benzoic acid. In some embodiments, the metal halogen is CuBr. In some embodiments, the 3,5-dibromo 4-hydroxybenzoic acid is reacted with NaOCH₃ and CuBr at a temperature from 80° C. to 120° C. to form 2-hydroxy 3,5-dimethoxy benzoic acid. In some embodiments, the 3,5-dichloro 4-hydroxybenzoic acid is reacted with NaOCH₃ and CuBr at a temperature from 80° C. to 120° C. to form 2-hydroxy 3,5-dimethoxy benzoic acid. In some embodiments, the 2-hydroxy 3,5-dimethoxy benzoic acid is decarboxylated with CuSO₄.

In still another aspect, a method for forming a reactive diluent comprising syringol methacrylate or syringol acrylate is provided. The method includes reacting the syringol disclosed herein with methacrylic anhydride or acrylic anhydride.

In still another aspect, a polymerizable composition is provided. The polymerizable composition includes a telechelic compound, a reactive diluent formed from the syringol disclosed herein, and an initiator. In some embodiments, the telechelic compound is present at a concentration of 30-70 wt % based on the total weight of the polymerizable composition. In some embodiments, the telechelic compound is present at a concentration of 40-60 wt % based on the total weight of the polymerizable composition. In some embodiments, the reactive diluent is present at a concentration of 30-70 wt % based on the total weight of the polymerizable composition. In some embodiments, the reactive diluent is present at a concentration of 40-60 wt % based on the total weight of the polymerizable composition. In some embodiments, the initiator is present at a concentration of 0.25-2.5 wt % based on the total weight of the polymerizable composition. In some embodiments, the initiator is present at a concentration of 0.5-2.0 wt % based on the total weight of the polymerizable composition. In some embodiments, the telechelic compound, reactive diluent, or both independently comprise methacrylate or acrylate reactive group. In some embodiments, the telechelic compound comprises polyurethane or polyester derivatives. In some embodiments, the reactive diluent is syringol methacrylate or syringol acrylate. In some embodiments, the initiator comprises a photoinitiator. In some embodiments, the photoinitiator comprises TPO-L (ethyl (2,4,6-trimethylbenzoyl)phenyl phosphinate).

In still another aspect, a polymeric material made from the polymerizable composition disclosed herein is provided.

In still another aspect, a method for forming an article by additive manufacturing is provided. The method includes providing a polymerizable composition disclosed herein, exposing the polymerizable composition to radiation to form a polymeric material, and fabricating the article with the polymeric material. In some embodiments, the method further comprises heating the polymerizable composition to a processing temperature. In some embodiments, the processing temperature is from about 50° C. to about 120° C. In some embodiments, the method further comprises receiving a file containing instructions for the fabrication of a dental appliance. In some embodiments, the additive manufacturing process is a 3D printing process. In some embodiments, the article is a medical device. In some embodiments, the medical device is a dental appliance. In some embodiments, the dental appliance comprises an oral sleep apnea appliance or a mouth guard. In some embodiments, the medical device is an orthodontic appliance. In some embodiments, the orthodontic appliance is a dental aligner, a palatal expander, an attachment, an attachment template, or a retainer.

In yet another aspect, a method of designing an orthodontic appliance disclosed herein is provided. The method includes determining a movement path to move one or more teeth from an initial arrangement to a target arrangement, determining a force system to produce movement of the one or more teeth along the movement path, determining a design for an orthodontic appliance configured to produce the force system, and generating instructions for fabrication of the orthodontic appliance incorporating the design.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1 is a flow diagram illustrating a first method for producing lignin-derived compounds from a biomass, in accordance with some embodiments of the present disclosure.

FIG. 2 is a flow diagram illustrating a second method for producing lignin-derived compounds from a biomass, in accordance with some embodiments of the present disclosure.

FIG. 3 is a flow diagram illustrating a third method for producing lignin-derived compounds from a biomass, in accordance with some embodiments of the present disclosure.

FIG. 4 is a flow diagram illustrating a fourth method for producing lignin-derived compounds from a biomass, in accordance with some embodiments of the present disclosure.

FIG. 5 is a flow diagram illustrating a fifth method for producing lignin-derived compounds from a biomass, in accordance with some embodiments.

FIG. 6 is a flow diagram illustrating a method for producing syringol from a tannic acid-rich biomass, in accordance with some embodiments.

FIG. 7 is a flow diagram providing a general overview of a method for fabricating and post-processing an additively manufactured object, in accordance with embodiments of the present disclosure.

FIG. 8 illustrates a representation example of an additive manufacturing device.

FIG. 9A shows a representative example of a tooth repositing appliance.

FIG. 9B depicts a tooth repositioning system including a plurality of appliances.

FIG. 9C is a flow diagram illustrating a method of orthodontic treatment using a plurality of appliances.

FIG. 10 is a flow diagram illustrating a method for designing an orthodontic appliance, in accordance with embodiments of the present disclosure.

FIG. 11 is a flow diagram illustrating a method for digitally planning an orthodontic treatment and/or design or fabrication of an appliance, in accordance with embodiments of the present disclosure.

FIG. 12 illustrates the effect of continuous oxygen feed on the yield of aromatic compounds during the depolymerization of lignin in walnut shells.

### DETAILED DESCRIPTION

Syringol can be produced from lignin and hydrolysable tannins. Due to their phenolic nature and widespread natural abundance, lignin and hydrolysable tannins are promising sustainable raw materials for syringol production.

Lignin, along with cellulose and hemicellulose, is one of the three primary components of lignocellulosic biomass, the most abundant renewable biomass on earth. Lignin is composed of guaiacyl, syringyl, and p-hydroxyphenyl monomers linked by C—O and C—C bonds, forming a complex three-dimensional structure. Upon depolymerization, lignin can directly produce syringol or yield syringaldehyde and syringic acid, which serve as platform chemicals for the production of syringol. Additionally, lignin depolymerization generates vanillin and vanillic acid, key fragrances used in the food, beverage, cosmetic, and pharmaceutical industries, as well as guaiacol, an important feedstock for various high-value chemicals. Lignin accounts for up to 60% by weight of typical biomass, making it a sustainable and promising feedstock for syringol production. Thus, utilizing lignocellulosic biomass as raw materials for the cost-effective production of syringol is highly desirable.

Similar to lignin, tannins are a family of naturally abundant polyphenols that can be extracted from biomass. There are three major classes of tannins, including hydrolysable tannins (also known as tannic acids), condensed tannins, and phlorotannins, which contain gallic acid, flavone, and phloroglucinol, respectively, as the base unit. Gallic acid is a known precursor for syringol synthesis. Therefore, utilizing tannic acid-rich biomass as a raw material presents a cost-effective approach for syringol production.

Preparation Syringol from Lignin-Rich Biomass

In one aspect, the present disclosure provides economically viable methods for producing lignin-derived compounds including one or more of syringol, vanillin, and guaiacol from lignin-rich biomass (i.e., lignocellulosic biomass). The methods may include providing biomass, pretreating the biomass, extracting lignin from the biomass, and depolymerizing lignin to yield high-valued phenolic compounds including one or more of syringol, vanillin, and guaiacol. Suitable lignin depolymerization processes include, but are not limited to, biological depolymerization, direct pyrolysis depolymerization, metal-catalyzed depolymerization, enzymatic depolymerization, base-catalyzed depolymerization, acid-catalyzed depolymerization, ionic liquids-assisted depolymerization, supercritical fluids-assisted depolymerization, electrochemical depolymerization, thermochemical depolymerization, and microwave-assisted depolymerization.

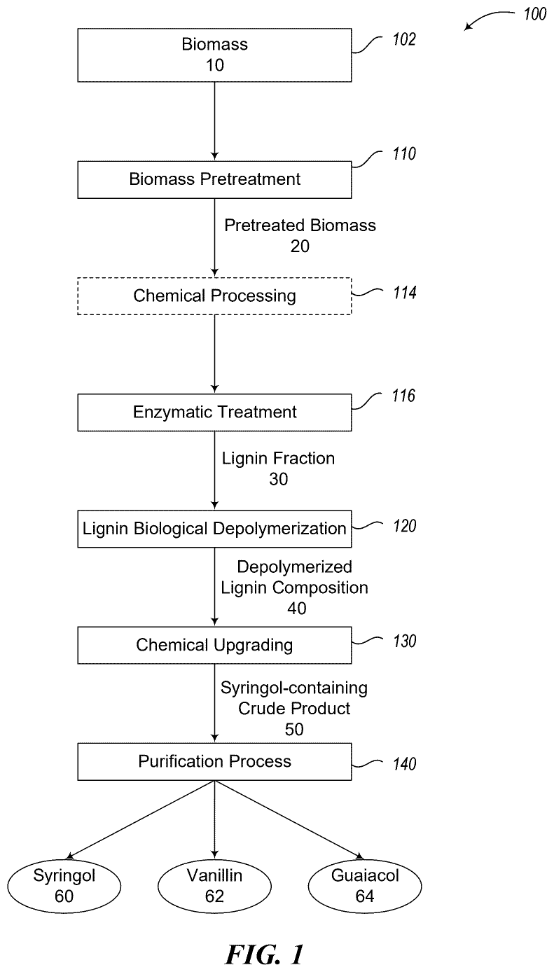

FIG. 1 illustrates a first method 100 for producing lignin-derived compounds including syringol 60, vanillin 62, and guaiacol 64 from a biomass 10, in accordance with some embodiments of the present disclosure. It is understood that the method 100 is merely an example and is not intended to limit the present disclosure beyond what is explicitly recited in the claims. Additional operations can be provided before, during, and after the method 100, and some operations described can be replaced, eliminated, or moved around for additional embodiments of the method.

Referring to FIG. 1, the method 100 includes operation 102, at which a biomass 10 is obtained for processing. The biomass 10 may be obtained from any common and/or high volume biomass source characterized by a high content of lignin. The biomass rich in lignin is also referred to as lignocellulose or lignocellulosic biomass. In some embodiments, the biomass 10 may have a lignin content of at least about 15%, at least about 20%, at least about 25%, at least about 30%, at least about 35%, at least about 40%, at least about 45%, or at least about 50%, by weight. In some embodiments, the biomass 10 may have a lignin content from about 30% to about 50%, by weight.

The biomass 10 may be received in any forms, including chips, pellets, cubes, and sawdusts. In some embodiments, the biomass 10 comprises a woody biomass obtained from limbs, tops, needles, leaves, barks, and other woody parts of hardwoods. Examples of hardwoods include, but are not limited to, oak, walnut, beech, maple, ash, and poplar. The woody biomass may include residues from food processing industries such as seed hulls and nutshells. The woody biomass may also be a by-product, residue or waste product of woody biomass, including woody biomass by-products, residues, and wastes from industries such as cellulosic bioethanol refineries, sawmills, timber harvest, construction, pulp and paper mills, and nut processing. In some embodiments, the woody biomass includes a nutshell. Examples of nutshells rich in lignin include, but are not limited to, shells from walnuts, pines, almonds, cashews, and the like. In some embodiments, the woody biomass includes walnut shells having a lignin content ranging from about 38% to 44%. In some embodiments, the biomass 10 comprises a non-woody biomass, for example, agricultural residue from annual crops. Examples of the non-woody biomasses include, but are not limited to, corn stover, wheat straw, rice stalk, barley straw, sugarcane bagasse, peanut shell, and soybean hull. In some embodiments, the biomass 10 comprises a bioengineered source obtained from microbes such as algae.

Next, at operation 110 of the method 100, the biomass 10 is pretreated by a mechanical means to reduce the size of the biomass 10 for ease of handling and increased surface volume ratio. The mechanical pretreatment facilitates more efficient and economical processing of downstream processes (e.g., lignin extraction, lignin depolymerization, etc.), but does not substantially affect the lignin, cellulose, and hemicellulose compositions of the biomass 10. In some embodiments, the biomass 10 is debarked, chipped, grinded, and/or milled to obtain a pretreated biomass 20. In some embodiments, the biomass 10 may be milled and/or ground using, for example, a hammer-mill and/or knife-mill. The pretreated biomass 20 comprises biomass particles having an average size of less than 10,000 μm, less than 9,000 μm, less than 8,000 μm, less than 7,000 μm, less than 6,000 μm, less than 5,000 μm, less than 4,000 μm, less than 3,000 μm, less than 1,000 μm, less than 400 μm, or less than 100 μm. In some embodiments, the average size of the biomass particles is in the range from 50 to 10,000 μm, for example, from 50 μm to 15.00 μm, from 70 μm to 1,200 μm, from 400 μm to 1,000 μm, or from 1,000 μm to 3,000 μm. In some embodiments, the average size of the biomass particles is from 70 μm to 1,200 μm, although particles outside this range are also contemplated. Various sizes of the biomass particles could be used depending on the scale of the reaction. In some embodiments, the size of the biomass particles is from 100 μm to 1,000 μm, from 100 μm to 800 μm, or from 200 μm to 600 μm.

In some embodiments, the pretreated biomass 20 may be dried to lower its moisture content. In some embodiments, after drying, the moisture content of the pretreated biomass 20 may be between 0 to 10% by weight. In some embodiments, the pretreated biomass 20 may contain less than 5% of moisture by weight.

In some embodiments, following the mechanical pretreatment, one or more operations (e.g., operation 114 (chemical processing) and operation 116 (enzymatic treatment)) may be performed to separate lignin from cellulose, hemicellulose and any non-cellulose and hemicellulose components in the pretreated biomass 20, producing a lignin fraction 30. The lignin fraction 30 is a purified source of lignin and, may in certain embodiments, comprise greater than 40%, 50%, 60%, 70%, 80%, 90%, 95%, or even 100% of pure lignin. The lignin fraction 30 may be subsequently directed through various processing operations that may include lignin depolymerization, chemical upgrading, and purification to afford syringol 60.

At operation 114 of the method 100, the pretreated biomass 20 is subject to a chemical processing, during which the pretreated biomass 20 may be extracted with one or more solvents to remove fats, oils, resins, waxes, and other extractables. Examples of suitable solvents includes, but are not limited to, water, ethanol, isopropanol, acetone, and mixtures thereof. Following the solvent extraction, the solvent(s) is removed and the pretreated biomass 20 is dried to give a solvent-free pretreated biomass. Operation 114 is optional, and may be omitted in some embodiments.

At operation 116 of the method 100, an enzymatic treatment is performed to extract lignin from the pretreated biomass 20. During the enzymatic treatment, the pretreated biomass 20 may be contacted with an enzyme composition under conditions effective to degrade lignocellulose. In some embodiments, the contacting can comprise soaking the pretreated biomass 20 into the enzyme composition. In some embodiments, the contacting can comprise spraying the enzyme composition onto the pretreated biomass 20. In some embodiments, the mixture of the pretreated biomass 20 and the enzyme composition may be stirred and agitated to ensure a complete mixing of the two.

The enzyme is capable of disrupting or degrading lignocellulose in the pretreated biomass 20, which leads to the separation of lignin from the other components of the lignocellulose such as cellulose and hemicellulose. Examples of enzymes that are suitable to break down the lignocellulose include, but are not limited to, laccases, cellobiohydrolases, endoglucanases, β-D-glucosidases, xylanases, arabinofuranosidases, acetyl xylan esterases, glucuronidases, mannanases, galactanases, arabinases, lignin peroxidases, manganese-dependent peroxidases, hybrid peroxidases, and ferulic acid esterases. In some embodiments, the weight ratio of the enzyme to the pretreated biomass 20 may be from 1:10 to 1:10,000, from 1:10 to 1:1,000, or from 1:10 to 1:100.

In some embodiments, the enzyme composition may include a mediator. In some embodiments, the enzyme is a laccase. Any mediators of laccase may be included to aid reactions catalyzed by the laccase leading to degradation of lignocellulose in the pretreated biomass 20. Examples of mediators include, but are not limited to, catechol, guaiacol, violuric acid, and 1-hydroxy-benzotriazole (HBT).

In some embodiments, the enzymatic treatment may be conducted at a temperature ranging from 25° C. to 75° C., for example, from 30° C. to 70° C., from 35° C. to 65° C., from 40° C. to 60° C., or from 45° C. to 55° C.

The enzymatic treatment may be performed for a period of time sufficient to separate lignin from cellulose and hemicellulose. In some embodiments, the enzymatic treatment may be performed for a period of time of 6 hours or more, 12 hours or more, 18 hours or more, 24 hours or more, 20 hours or more, or 36 hours or more. The lignin fraction 30 formed after the enzymatic treatment may be in the form of suspension which contains water, acid such as formic acid, acetic acid or sulfuric acid, alcohol or other liquid, or in the form of cake, lump or the like. In some embodiments, the lignin fraction may be washed to remove the impurities.

Although enzyme extraction is described above, in some embodiments, a solvent-such as an ionic liquid—may be used to extract lignin from the pretreated biomass 20. The ionic liquid may be 1-ethyl-3-methylimidazolium acetate ([C2mim] [OAc]). In some embodiments, the solvent extraction of lignin may be facilitated by ultrasound or microwave.

Following the enzymatic treatment, at operation 120 of the method 100, the lignin fraction 30 undergoes a lignin depolymerization process, during which lignin is depolymerized to produce a depolymerized lignin composition 40 comprising a mixture of low-molecular-weight aromatic compounds. In some embodiments, the depolymerized lignin composition 40 may comprise syringol, syringaldehyde, syringic acid, vanillin, vanillic acid, guaiacol, p-hydroxybenzaldehyde, and p-hydroxybenzoic acid.

In some embodiments, the lignin depolymerization is a biological depolymerization process using a biocatalyst. In some embodiments, the biocatalyst comprises a lignolytic microbe, including fungi, bacteria, and achaca, and engineered microbes that are designed to depolymerize lignin. In some embodiments, the lignolytic bacteria include E. coli, bacillus, Streptomyces, and Rhodococcus species. In some embodiments, the lignolytic bacteria includes anaerobic microorganisms selected from Neocallimastigomycetes, Aerobacter, Aeromonas, Alcaligenes, Bacillus, Bacteroides, Clostridium, Escherichia, Klebsiella, Leptospira, Micrococcus, Neisseria, Paracolobacterium, Proteus, Pseudomonas, Rhodopseudomonas, Sarcina, Serratia, and Streptococcus.

In some embodiments, the biological depolymerization process may be conducted at temperatures between 10° C. to 70° C., between 35° C. to 65° C., or between 37° C. to 60° C.

Following the lignin depolymerization, at operation 130 of the method 100, the depolymerized lignin composition 40 is subject to a chemical upgrading process to produce a syringol-containing crude product 50.

In some embodiments, the chemical upgrading process is a two-step process, during which the syringaldehyde (3,5-dimethoxy-4-hydroxybenzaldehyde) is first oxidized to produce syringic acid, which is in turn transformed into syringol through decarboxylation.

In some embodiments, syringaldehyde is oxidized by oxygen with bis(methoxypropyl) ether as a promotor and solvent. In some embodiments, oxidation of syringaldehyde may be accomplished by first dissolving the depolymerized lignin composition 40 in bis(methoxypropyl) ether, and introducing an oxidation agent into the resulting solution. In some embodiments, the oxidation agent can include oxygen gas, air, or oxygen-enriched air. The oxidation gas can be introduced alone or mixed with one or more carrier gases such as nitrogen, argon, or helium. In some embodiments, the oxidation agent includes a mixture of oxygen and nitrogen gas with an amount of oxygen gas ranging from 5% to 10% by weight. In some embodiments, the oxygen gas in the oxidation agent is about 8% by weight. The pressure of the oxidation agent may range from 100 psi to 300 psi. In some embodiments, the pressure of the oxidation agent is about 200 psi. Other examples of oxidation agents that may be used to convert syringaldehyde to syringic acid include, but are not limited to, a halite such as sodium chlorite, sodium bromite, or calcium chlorite, a hypohalite such as sodium hypochlorite or sodium hypobromite, sodium bromate, silver nitrate, Mn(OAc)₃, Mn₂O₃, MnO₂, Mn(NO₃)₂, Mg(OAc)₂, iodobenzene diacetate [PhI(OAc)₂], t-BuOCl, trichloroisocyanuric acid, and tert-butyl hydroperoxide

The decarboxylation of syringic acid may be performed with or without a decarboxylation catalyst. In some embodiments, the decarboxylation reaction that converts syringic acid into syringol may be carried out by contacting the depolymerized lignin composition 40 with a decarboxylation catalyst. In some embodiments, the decarboxylation catalyst is a copper-based catalyst selected from CuCl, Cu(NO₃)₂, Cu₂O, CuO, Cu, CuSO₄, Cu(OAc)₂, and CuCl₂. The amount of decarboxylation catalyst may range from 0.05 to 3 eq. relative to syringic acid. In some embodiments, the amount of decarboxylation catalyst is about 0.1, 1.1, or 2.5 eq. relative to syringic aid. The reaction may be carried out at an elevated temperature of about 120° C. or more, 150° C. or more, 180° C. or more, 200° C. or more, or 250° C. or more. The reaction times may be about 24 hours or less, 20 hours or less, 15 hours or less, 10 hours or less, 8 hours or less, or 6 hours or less. In some embodiments, the decarboxylation reaction is conducted at 190° C. or 200° C. for about 6 hours. In some embodiments, the decarboxylation reaction is conducted at 170° C. for about 24 hours.

In some embodiments, the chemical upgrading process is a single step process, where the depolymerized lignin composition 40 is directly treated with a decarboxylation catalyst. In some embodiments, the depolymerized lignin composition 40 is treated with CuSO₄ at 170° C. for 24 hours to provide a syringol-containing crude product 50.

After chemical upgrading, at operation 140 of the method 100, the syringol-containing crude product 50 is subject to a purification step, yielding syringol 60. In some embodiments, after the purification step, vanillin 62 and guaiacol 64 can also be produced.

In some embodiments, the purification step may encompass a selective solvent extraction process using a basic aqueous solution comprising a base and an immiscible organic solvent such as methyl tert-butyl ether (MTBE), heptane, ethyl acetate, methylcyclohexane, toluene, ethyl ether, pentane, or the like. The pH value of the basic aqueous solution may be adjusted by potassium oxide (KOH) or sodium oxide (NaOH). In some embodiments, the selective solvent extraction may be carried out in two stages using the above basic aqueous solutions of different pH values, yielding a syringol-enriched bio-oil. For example, in the first stage, the syringol-containing crude product 50 is extracted with a first basic aqueous solution having a pH of 13.0 to 13.5. At this pH range, the organic phase extracts a portion of impurities which typically consist of hydrocarbon aromatics, while syringol stays in the aqueous phase. Subsequently, the aqueous phase is collected and extracted with a second basic aqueous solution having a pH of 9.5 in the second stage. At the pH of 9.5, syringol is repelled from the aqueous phase and extracted into the organic phase. The organic phase is then separated, dried, and concentrated, obtaining the syringol-enriched bio-oil.

Subsequently, the syringol-enriched bio-oil is further purified by fraction distillation using a distillation column. In some embodiments, the distillation column has ten plates. In some embodiments, the distillation may be carried out with a temperature at the bottom of the distillation column ranging from about 50° C. to 250° C. or from about 115° C. to 140° C. The distillation can be carried out under atmospheric pressure or reduced pressure. In some embodiments, the pressure is from 0.02 psi to 15 psi, or from 0.05 psi to 10 psi. The fraction distillation separates syringol from the other phenolic compounds in the syringol-enriched bio-oil, yielding syringol 60. In some embodiments, the fraction distillation also separates vanillin 62 and guaiacol 64 from other phenolic compounds in the syringol-enriched bio-oil.

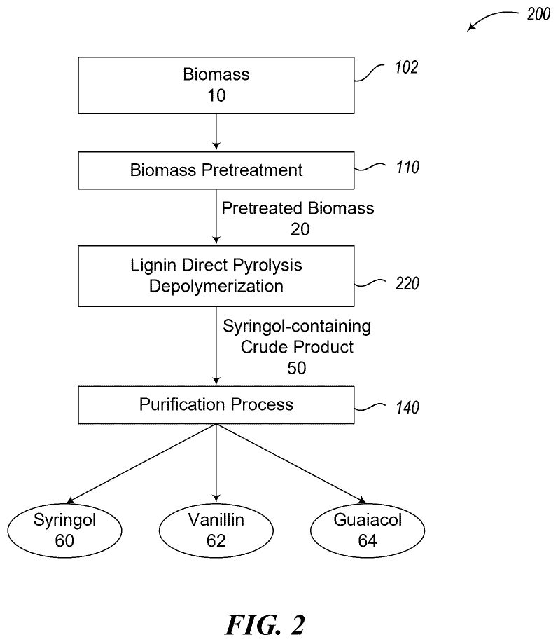

FIG. 2 is a second method 200 for producing lignin-derived compounds including syringol 60, vanillin 62, and guaiacol 64 from a biomass 10, in accordance with some embodiments of the present disclosure. It is understood that the method 200 is merely an example and is not intended to limit the present disclosure beyond what is explicitly recited in the claims. Additional operations can be provided before, during, and after the method 200, and some operations described can be replaced, eliminated, or moved around for additional embodiments of the method.

Compared to the first method 100, in the second method 200, the enzymatic treatment step (operation 116) that separates the lignin from the cellulose and/or hemicellulose in the pretreated biomass 20 is omitted. Accordingly, in the method 200, after performing operations 102 and 110, the pretreated biomass 20 directly undergoes a depolymerization process by direct pyrolysis at operation 220. Eliminating the lignin separation operation prior to the lignin depolymerization operation provides substantial benefits as it eliminates a significant portion of the waste component from the process and eliminates the need to purify the lignin fraction before the lignin depolymerization process. As a result, the manufacturing cost can be reduced.

Additionally, unlike method 100, where lignin decomposition occurs through biological catalysts at operation 120, method 200 employs a direct pyrolysis depolymerization process at operation 220 to break down the lignin in the pretreated biomass 20, directly producing syringol as the major component. Consequently, method 200 eliminates the need for any chemical upgrading steps.

In some embodiments, the lignin direct pyrolysis depolymerization process is carried out at elevated temperatures under an inert atmosphere, with the absence of oxygen. This inert atmosphere can be established, for example, by flowing an inert gas, such as nitrogen or argon, through the reactor. The pyrolysis is carried out at high temperatures sufficient to cause cracking of the ether and carbon-carbon bonds in the lignin, resulting in the syringol-containing crude product 50. Consequently, the chemical upgrading operation (i.e., operation 130) in method 100 can be omitted.

In some embodiments, the direct pyrolysis of the pretreated biomass 20 is carried out at temperatures ranging from 500° C. to 600° C. The direct pyrolysis is performed for a duration long enough to convert the majority or substantially all of lignin in the pretreated biomass 20 into the syringol-containing crude product 50. In some embodiments, the direct pyrolysis is performed from about 2 minutes to about 40 minutes, from about 5 minutes to about 40 minutes, or from about 5 minutes to about 30 minutes. In other embodiments, the direct pyrolysis is performed for a period of less than about 20 minutes.

Following the direct pyrolysis depolymerization process, the resulting syringol-containing crude product 50 is purified by performing operation 140 of the method 100, producing syringol 60, vanillin 62, and guaiacol 64.

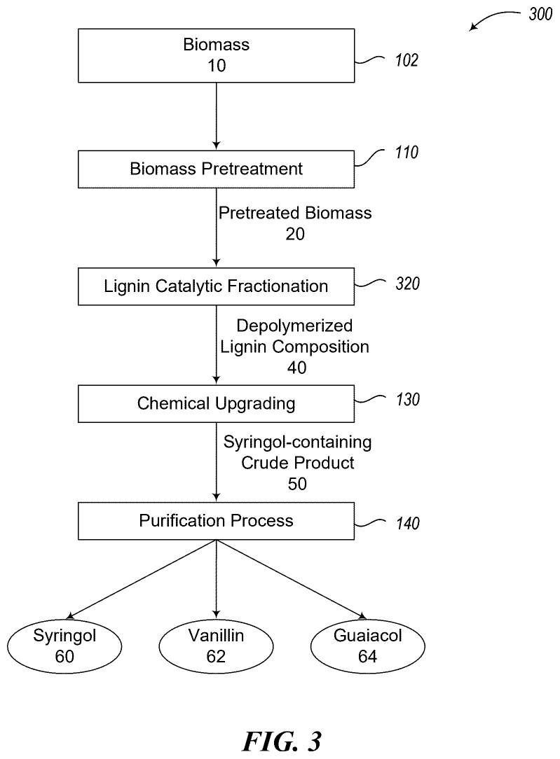

FIG. 3 is a third method 300 for producing lignin-derived compounds including syringol 60, vanillin 62, and guaiacol 64 from a biomass 10, in accordance with some embodiments of the present disclosure. It is understood that the method 300 is merely an example and is not intended to limit the present disclosure beyond what is explicitly recited in the claims. Additional operations can be provided before, during, and after the method 300, and some operations described can be replaced, eliminated, or moved around for additional embodiments of the method.

Compared to the second method 200, which employs a direct pyrolysis depolymerization process to decompose lignin in the pretreated biomass 20 at operation 220, the third method 300 utilizes a catalytic fractionation process for lignin depolymerization at operation 320. In method 300, after performing operations 102 and 110 to provide the pretreated biomass 20, the lignin in the pretreated biomass 20 is depolymerized by a catalytic fractionation process (operation 320).

Operation 320 generates syringic acid and syringaldehyde, which then undergo chemical upgrading to produce syringol.

In some embodiments, the process is a heterogeneously catalyzed lignin depolymerization process, where the lignin catalytic fractionation may be carried out in the presence of a catalyst supported on a support material. The lignin depolymerization catalyst can be a transition metal such as zinc (Zn), iron (Fe), cobalt (Co), copper (Cu), manganese (Mn), magnesium (Mg), titanium (Ti), chromium (Cr), and nickel (Ni) or oxides thereof. In some embodiments, the lignin depolymerization catalyst includes a transition metal oxide such as, for example, ZnO, CoO, Co₃O₄, CuO, MnO, or Fe₂O₃. The support material may include a metal oxide. In some embodiments, the support material may include a silica, alumina, titania, zirconia, magnesium oxide, silica-alumina, carbon black, zeolites, and mixtures thereof. In some embodiments, the depolymerization catalyst may contain from about 0.1% to about 30% by weight or from about 3% to about 20% by weight, of metal, based on the total weight of the catalyst.

To increase the surface areas of the depolymerization catalyst, a porous support material having a high surface area is used. In some embodiments, the lignin depolymerization catalyst may be characterized by a Brunauer-Emmett-Teller (BET) surface area ranging from about 1.0 m²/g to about 500 m²/g as measured by gas adsorption analysis. Alternatively, the lignin depolymerization catalyst may be characterized by a BET surface area ranging from about 8 m²/g to about 250 m²/g. In still further examples, the lignin depolymerization catalyst may be characterized by a BET surface area of about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, about 110 m²/g, about 120 m²/g, about 130 m²/g, about 140 m²/g, about 150 m²/g, about 180 m²/g, or about 200 m²/g.

In some other embodiments, a transition metal-based catalyst can be dissolved in a solvent to act as a homogenous catalyst, facilitating lignin depolymerization. Examples of suitable lignin depolymerization catalysts include, but are not limited to, zinc sulfate (ZnSO₄), zinc acetylacetonate (Zn(C₅H₇O₂)₂), zinc acetate (Zn(CH₃COO)₂), cobalt chloride (CoCl₂), cobalt acetylacetonate (Co(C₅H₇O₂)₂), cobalt acetate (Co(CH₃COO)₂), copper sulfate (CuSO₄), copper acetate (Cu(CH₃COO)₂), manganese sulfate (MnSO₄), manganese acetylacetonate (Mn(C₅H₇O₂)₂), manganese acetate (Mn(CH₃COO)₂), iron acetylacetonate (Fe(C₅H₇O₂)₃), iron chloride (FeCl₃), ferric citrate, ferric acetate (Fe(CH₃COO)₃), and iron phthalocyanine (FePc). The solvent may be any polar solvent including, but not limited to, water, alcohols such as methanol or ethanol, acetone, acetonitrile, anisole, cyclic ketones such as cyclopentanone, cyclohexanone, cycloheptanone or cyclooctenone, methylene chloride (MEC), perchloroethylene (PCE), trichloroethylene (TCE), polyethylene glycol (PEG-200 or PEG-400), sulfolane, γ-valerolactone (GVL), and N-methylpyrrolidone (NMP). In some embodiments, the solvent is a mixed solvent comprising water and acetonitrile. In some embodiments, a supercritical solvent such as supercritical water, methanol, ethanol, or propanol may be used.

The lignin depolymerization catalyst may be added in at least catalyst amounts. In some embodiments, the amount of the lignin depolymerization catalyst is 0.05 mol % or more, 0.1 mol % or more, 0.2 mol % or more, 0.3 mol % or more, 0.4 mol % or more, 0.5 mol % or more, 1 mol % or more, 2 mol % or more, 3 mol % or more, 4 mol % or more, 5 mol % or more, 6 mol % or more, 7 mol % or more, or 10 mol % or more, based on the total weight of the pretreated biomass 20. In some embodiments, the amount of the lignin depolymerization catalyst is 90 mol % or less, 50 mol % or less, 25 mol % or less, 15 mol % or less, or 12 mol % or less, based on the total weight of the pretreated biomass 20. In some embodiments, the amount of lignin depolymerization catalyst is from 0.05 mol % to 2 mol % based on the total weight of the pretreated biomass 20.

Compared to the direct pyrolysis depolymerization process, the catalytic fractionation process can be conducted at much milder reaction conditions with a reaction temperature below 200° C. and a pressure below 600 psi. In some embodiments, the catalytic depolymerization may be conducted by mixing the pretreated biomass 20 and the lignin depolymerization catalyst in a solvent (e.g., acetone, acetonitrile, MEC, PCE, and/or TCE, etc.). The resulting mixture is heated to 190° C. and maintained at 190° C. for a period of time in the presence of oxygen. The reaction time may range from 1 hour or more, from 2 hours or more, from 3 hours or more, from 4 hours or more, from 5 hours or more, from 6 hours or more, or from 7 hours or more. In some embodiments, the mixture of the pretreated biomass 20 and the catalyst may be heated to 190° C. and then maintained at 190° C. and 500 psi for 6 h in the presence of oxygen, yielding the depolymerized lignin composition 40. In some embodiments, the oxygen is from an oxygen source consisting of O₂ gas balanced with N₂ gas, while in other embodiments, the oxygen source is air. The O₂ concentration in the oxygen source can vary, influencing product selectivity. Higher O₂ content favors syringic acid production, whereas lower O₂ content favors syringaldehyde formation. In some embodiments, the oxygen source contains 2 vol %, 4%, 6 vol %, 8 vol %, or 10 vol % oxygen gas. In some embodiments, the oxygen source may be supplied to the reactor in a batch, such that when oxygen in the reactor is depleted, the lignin catalytic depolymerization reaction stops. In some embodiments, the oxygen source can be fed continuously or semi-continuously, controlled by a regulator and valves, into the reactor. High yields are achieved when oxygen in the reactor can be replenished. The lignin catalytic depolymerization generates the depolymerized lignin composition 40, which contains syringic acid and vanillin. In some embodiments, the yield for syringic acid is 1 wt %, 2 wt %, 3 wt %, 4 wt %, or 5 wt %, and the yield for vanillin is 1 wt %, 2 wt %, 3 wt %, 4 wt %, or 5 wt %.

Following the lignin catalytic depolymerization, the depolymerized lignin composition 40 may undergo chemical upgrading (operation 130) and purification (operation 140) of the method 100 to obtain syringol 60, vanillin 62 and guaiacol 64.

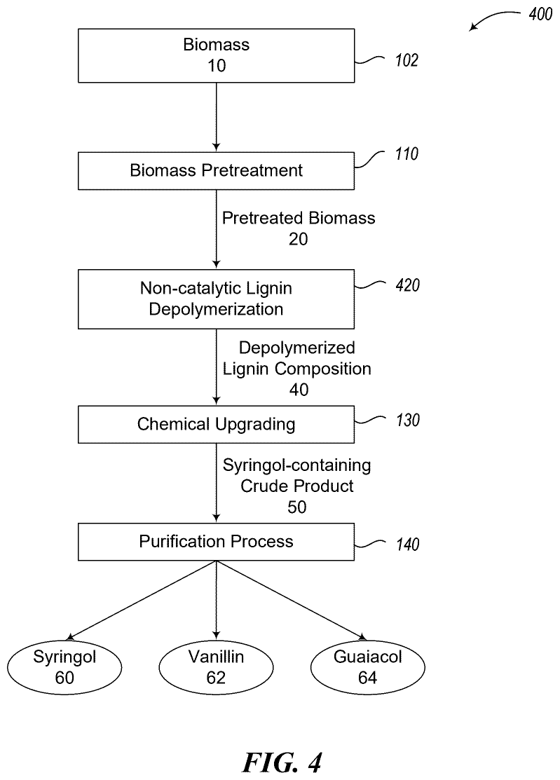

FIG. 4 is a fourth method 400 for producing lignin-derived compounds including syringol 60, vanillin 62, and guaiacol 64 from a biomass 10, in accordance with some embodiments of the present disclosure. It is understood that the method 400 is merely an example and is not intended to limit the present disclosure beyond what is explicitly recited in the claims. Additional operations can be provided before, during, and after the method 400, and some operations described can be replaced, eliminated, or moved around for additional embodiments of the method.

Compared to the third method 300, which utilizes a lignin depolymerization catalyst to break down lignin in the pretreated biomass 20 in the presence of oxygen, the fourth method 400 employs a non-catalytic approach. In method 400, lignin in the pretreated biomass 20 undergoes depolymerization at a temperature below its typical pyrolysis range of 500° C. to 600° C., in the presence of oxygen, without the need for a depolymerization catalyst.

In method 400, after providing a biomass 10 at operation 102, operation 110 is performed to produce pretreated biomass 20. The method 400 then proceeds to operation 420, where non-catalytic lignin depolymerization is carried out in the presence of oxygen at an elevated temperature. In some embodiments, at operation 420, the non-catalytic depolymerization of lignin may be conducted by treating the pretreated biomass 20 under reaction conditions similar to the conditions described above in operation 320. For example, in some embodiments, the non-catalytic depolymerization of lignin is carried out at a reaction temperature below 200° C., for example, at 190° C., 180° C. or 170° C. and a pressure below 600 psi, for example, at 500 psi, 400 psi or 300 psi, in the presence of oxygen. In some embodiments, the non-catalytic depolymerization may be conducted by mixing the pretreated biomass 20 with a solvent, and the resulting mixture is heated to 190° C. in the presence of an oxygen source. The solvent may be any polar solvent including, but not limited to, water, alcohols such as methanol or ethanol, acetone, acetonitrile, anisole, cyclic ketones such as cyclopentanone, cyclohexanone, cycloheptanone or cyclooctenone, methylene chloride (MEC), perchloroethylene (PCE), trichloroethylene (TCE), polyethylene glycol (PEG-200 or PEG-400), sulfolane, γ-valerolactone (GVL), and N-methylpyrrolidone (NMP). In some embodiments, the solvent is a mixed solvent comprising water and acetonitrile. In some embodiments, a supercritical solvent such as supercritical water, methanol, ethanol, or propanol may be used. The reaction time may range from 1 hour or more, from 2 hours or more, from 3 hours or more, from 4 hours or more, from 5 hours or more, from 6 hours or more, or from 7 hours or more. In some embodiments, the pretreated biomass 20 may be heated to 190° C. and then maintained at 190° C. and 500 psi for 6 h in the presence of oxygen, yielding the depolymerized lignin composition 40. The depolymerized lignin composition 40 primarily contains syringic acid and syringaldehyde as the major components, while syringol is not present. In some embodiments, the oxygen source of the reaction is O₂ gas balanced with N₂ gas. The concentration of the O₂ gas can vary. Depending on the amount of O₂ present, the product selectivity can change accordingly. More O₂ content favors syringic acid production while less O₂ favors syringaldehyde. In some embodiments, the oxygen source contains 2 vol %, 4%, 6 vol %, 8 vol %, or 10 vol % oxygen gas. In some embodiments, the oxygen source is air. In some embodiments, the oxygen source is introduced into the reactor in a batch-wise manner, with additional oxygen added once the oxygen in the initial supply is depleted and the reaction slows or stops. In some embodiments, as oxygen is consumed during a reaction cycle, the reactor is opened, and the headspace of the reactor is replenished with the oxygen source to start the next reaction cycle. The reaction may be carried out for at least four cycles, with oxygen being added at least four times throughout the process. In some embodiments, operation 420 produces syringic acid with a yield of 1 wt %, 2 wt %, 3 wt %, 4 wt %, or 5 wt %; syringaldehyde with a yield of 0.5 wt %, 1 wt %, 2 wt %, 3 wt %, or 4 wt %; vanillic acid with a yield of 0.5 wt %, 1 wt %, 2 wt %, 3 wt %, or 4 wt %; and vanillin with a yield of 0.5 wt %, 1 wt %, 2 wt %, 3 wt %, or 4 wt %. A higher oxygen content in air, compared to a nitrogen-balanced oxygen source, results in faster kinetics and increased yields of syringic acid, syringaldehyde, vanillic acid, and aniline in the batch operation. In some embodiments, after four reaction cycles using a nitrogen-balanced gas containing 8 vol % of oxygen is used as the oxygen source, the depolymerized lignin composition 40 comprises 1.3 wt % of syringic acid, 0.9 wt % of syringaldehyde, 0.6 wt % of vanillic acid, and 0.6 wt % of vanillin. When air that contains approximately 21 vol % oxygen is used as the oxygen source, the depolymerized lignin composition 40 comprises 2.3 wt % of syringic acid, 1.0 wt % of syringaldehyde, 1.5 wt % of vanillic acid, and 0.75 wt % of vanillin.

Following the lignin non-catalytic depolymerization, the depolymerized lignin composition 40 may undergo chemical upgrading (operation 130) and purification (operation 140) of the method 100 to obtain syringol 60, vanillin 62 and guaiacol 64.

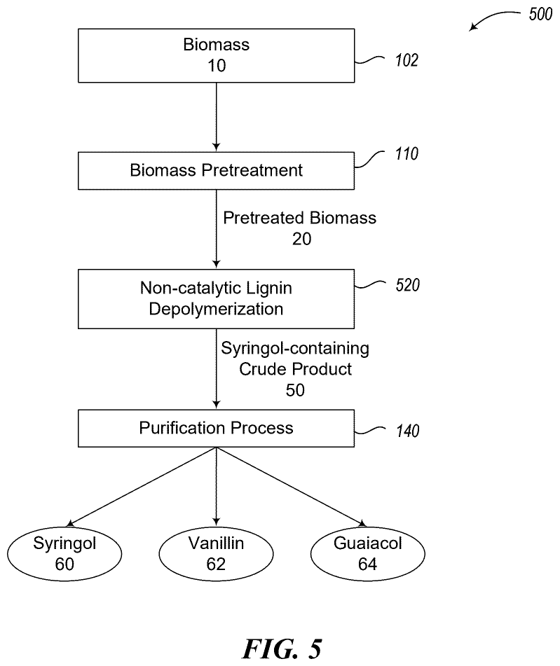

FIG. 5 is a fifth method 500 for producing lignin-derived compounds including syringol 60, vanillin 62, and guaiacol 64 from a biomass 10, in accordance with some embodiments of the present disclosure. It is understood that the method 500 is merely an example and is not intended to limit the present disclosure beyond what is explicitly recited in the claims. Additional operations can be provided before, during, and after the method 500, and some operations described can be replaced, eliminated, or moved around for additional embodiments of the method.

Compared to the fourth method 400, where the oxygen source in the non-catalytic lignin depolymerization process is introduced to the reactor in a batch-wise manner, producing syringic acid and syringaldehyde as the major components instead of syringol, the fifth method 500 introduces the oxygen source in a continuous manner during the non-catalytic lignin depolymerization process (operation 520), resulting in syringol as the primary product.

In method 500, after providing a biomass 10 at operation 102, operation 110 is performed to produce pretreated biomass 20. The method 500 then proceeds to operation 520, where non-catalytic lignin depolymerization is carried out with continuous flow of oxygen into the reactor.

In some embodiments, at operation 520, the non-catalytic depolymerization of lignin may be carried out at elevated temperatures and above ambient pressures with the continuous follow of oxygen. For example, in some embodiments, the non-catalytic depolymerization of lignin is carried out at a reaction temperature below 200° C., for example, at 190° C., 180° C. or 170° C. and a pressure below 600 psi, for example, at 500 psi, 400 psi or 300 psi, with the continuous follow of oxygen. In some embodiments, the non-catalytic depolymerization may be conducted by mixing the pretreated biomass 20 with a solvent, and the resulting mixture is heated to 190° C. with the continuous flow of oxygen by supplying an oxygen source in the reactor. The solvent may be any polar solvent including, but not limited to, water, alcohols such as methanol or ethanol, acetone, acetonitrile, anisole, cyclic ketones such as cyclopentanone, cyclohexanone, cycloheptanone or cyclooctenone, methylene chloride (MEC), perchloroethylene (PCE), trichloroethylene (TCE), polyethylene glycol (PEG-200 or PEG-400), sulfolane, γ-valerolactone (GVL), and N-methylpyrrolidone (NMP). In some embodiments, the solvent is a mixed solvent comprising water and acetonitrile. In some embodiments, a supercritical solvent such as supercritical water, methanol, ethanol, or propanol may be used. The reaction time may range from 1 hour or more, from 2 hours or more, from 3 hours or more, from 4 hours or more, from 5 hours or more, from 6 hours or more, from 7 hours or more, from 8 hours or more, from 10 hours or more, from 12 hours or more, from 14 hours or more, or from 16 hours or more. In some embodiments, the pretreated biomass 20 may be heated to 190° C. and then reacted at 190° C. and 500 psi for 16 h, yielding a syringol-containing crude product 50 with syringol as the primary component. In some embodiments, the syringol-containing crude product 50 may include 0.5 wt % to 20 wt % of syringol. In some embodiments, the syringol-containing crude product 50 may further comprise syringic acid, syringaldehyde, vanillic acid and vanillin. In some embodiments, the oxygen source of the reaction is O₂ gas balanced with N₂ gas. In some embodiments, the oxygen source contains 2 vol %, 4%, 6 vol %, 8 vol %, or 10 vol % oxygen gas. In some embodiments, the oxygen source is air. In some embodiments, the oxygen source is continuously flowed through the reactor at a rate ranging from 0.2 L/min to 5 L/min, for example, from 0.3 L/min to 1 L/min. In some embodiments, the oxygen source flow rate is 0.2 L/min, 0.3 L/min, 0.4 L/min, 0.5 L/min, 0.6 L/min, 0.7 L/min, 0.8 L/min, 0.9 L/min, 1 L/min, 2 L/min, 3 L/min, 4 L/min, or 5 L/min.

In method 500, because operation 520 breaks down the lignin in the pretreated biomass 20, directly producing syringol as the primary component. Consequently, method 500 eliminates the need for any chemical upgrading steps. Accordingly, following the non-catalytic lignin depolymerization process, the resulting syringol-containing crude product 50 is purified by performing operation 140 of the method 500, producing syringol 60, vanillin 62, and guaiacol 64.

Preparation Syringol from Tannic Acid-Rich Biomass

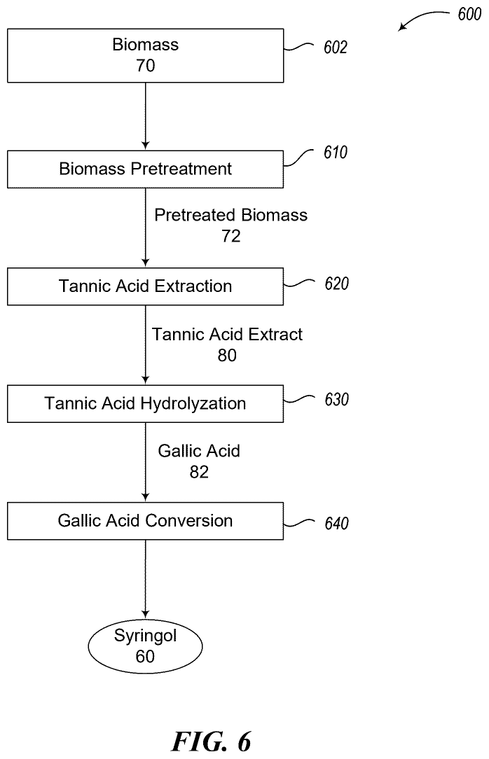

In one aspect, the present disclosure provides methods for producing syringol from a tannic acid-rich biomass. FIG. 6 illustrates a method 600 for producing syringol from a tannic acid-rich biomass 70, in accordance with some embodiments of the present disclosure. It is understood that the method 600 is merely an example and is not intended to limit the present disclosure beyond what is explicitly recited in the claims. Additional operations can be provided before, during, and after the method 600, and some operations described can be replaced, eliminated, or moved around for additional embodiments of the method.

Referring to FIG. 6, the method includes operation 602, where a tannic acid-rich biomass 70 is provided for processing. The tannic acid-rich biomass 70 may include gallnuts (e.g., oak gallnuts), tree barks (e.g., tara tree barks or oak barks), leaves (e.g., sumac leaves or tea leaves), husks (e.g., pomegranate husks), and pods (e.g., tara pods) from plants. The tannic acid-rich biomass 70 may contain tannic acids having 4-12 galloyl moieties, for example, e.g., 4-10 galloyl moieties, 5-10 galloyl moieties, 5-12 galloyl moieties, or 8-12 galloyl moieties. In some embodiments, the tannic acid-rich biomass 70 may be gallnuts containing a tannic acid with 4 galloyl moiety.

Next, at operation 610 of the method 600, the biomass is pretreated by a mechanical means to reduce the size of the biomass 70. The mechanical pretreatment facilitates more efficient and economical processing of tannic acid extraction. In some embodiments, the biomass 70 is grinded to afford a pretreated biomass 72. The pretreated biomass 72 comprises biomass particles having an average size less than 10,000 μm, 9,000 μm, 8,000 μm, 7,000 μm, 6,000 μm, 5,000 μm, 4,000 μm, 3,000 μm, 1,000 μm, 400 μm, or 100 μm. In some embodiments, the average size of the biomass particles is in the range from 50 to 10,000 μm, for example, from 50 to 15.00 μm, from 70 to 1,200 μm, from 400 to 1,000 μm, or from 1,000 to 3,000 μm. In some embodiments, the average size of the biomass particles is from 70 μm to 1,200 μm, although particles outside this range are also contemplated. Various sizes of the biomass particles could be used depending on the scale of the reaction. In some embodiments, the size of the biomass particles is from 100 μm to 1,000 μm, from 100 μm to 800 μm, or from 200 μm to 600 μm.

In some embodiments, the pretreated biomass 72 may be dried to lower its moisture content. In some embodiments, after drying, the moisture content of the pretreated biomass 72 may be between 0 to 10% by weight. In some embodiments, the pretreated biomass 72 may contain less than 5% of moisture by weight.

Next, at operation 620, a tannic acid extract 80 is obtained from the pretreated biomass 72. In some embodiments, the tannic acid extract 80 is extracted from the pretreated biomass 72 by contacting the pretreated biomass 72 with a solvent. Examples of solvents include, but are not limited to, water, acetone, methyl ethyl ketone, ethyl acetate, methyl acetate, ethanol, isopropanol, 1,4-dioxane, hexane, and tetrahydrofuran. In some embodiments, to improve the exaction yield, operation 620 may be performed at room temperature or at elevated temperatures. In some embodiments, operation 620 is carried out at a temperature ranging from 20° C. to 60° C. After extraction, the tannic acid extract 80 is separated from the solid phase by filtration, centrifuge, or other suitable liquid-solid separation techniques. The solvent is then removed, for example, by distillation, providing the tannic acid extract 80.

Next, at operation 630, tannic acid in the tannic acid extract 80 may be hydrolyzed to afford gallic acid 82. In some embodiments, the hydrolysis of the tannic acid may be carried out using hot water, without the addition of other substances or chemicals, or using weak acids having pH values of 4 to less than 7 or weak bases having pH values of greater than 7 to 9. In some embodiments, the hydrolysis of the tannic acid may be performed by enzymes such as tannase (i.e., tannin acyl hydrolase or polyphenol oxidase), which specifically break down the tannic acid extract 80 into gallic acid 82. The gallic acid 82 is then separated from the tannic acid extract 80 using distillation.

Next, at operation 640, the gallic acid 82 is converted to syringol 60 by decarboxylation, which then undergoes methylation. In some embodiments, synthesis of syringol 60 using gallic acid 82 as the starting material is illustrated in Scheme 1.

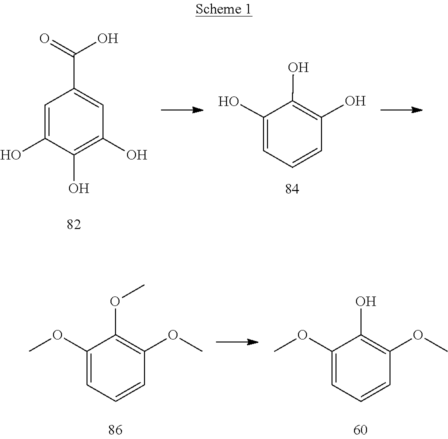

First, gallic acid 82 is decarboxylated to form pyrogallol 84. In some embodiments, the decarboxylation of gallic acid 82 may be performed with or without a decarboxylation catalyst. In some embodiments, the decarboxylation reaction that converts gallic acid 82 into pyrogallol 84 may be carried out by contacting the gallic acid 82 with a decarboxylation catalyst. In some embodiments, the decarboxylation catalyst is a copper-based catalyst selected from CuCl, Cu(NO₃)₂, Cu₂O, CuO, Cu, CuSO₄, Cu(OAc)₂, and CuCl₂. The amount of decarboxylation catalyst may range from 0.05 to 3 eq. relative to gallic acid 82. In some embodiments, the amount of decarboxylation catalyst is about 0.1, 1.1, or 2.5 eq. relative to gallic acid 82. The reaction may be carried out at elevated temperatures of about 120° C. or more, 150° C. or more, 180° C. or more, 200° C. or more, or 250° C. or more. The reaction times may be about 24 hours or less, 20 hours or less, 15 hours or less, about 10 hours or less, 8 hours or less, or 6 hours or less. In some embodiments, the decarboxylation reaction is conducted at 190° C. or 200° C. for about 6 hours. In some embodiments, the decarboxylation reaction is conducted at 170° C. for about 24 hours.

Next, 1,2,3-trimethoxybenzene 86 is synthesized from pyrogallol 84 via methylation. In some embodiments, the methylation of pyrogallol 84 is carried out by reacting pyrogallol 84 with a methylation reagent, for example, methyl iodide, and K₂CO₃ in a polar solvent, such as acetone, dimethylsulfate, methyl tosylate, dimethyl carbonate, or trimethyl phosphate, at 55° C. for over 90% yield. It is noted that other reagents and reaction conditions can be used to afford 1,2,3-trimethoxybenzene 86 from pyrogallol 84. For example, Li₂CO₃, NaH, KHCO₃, Cs₂CO₃, or the like can be used as a base. Any alkyl halides can be also used such as CH₃Br or CH₃Cl. Further, alkyl sulfonates can be used such as methyl p-toluenesulfonate, methyl methanesulfonate, or the like.

Subsequently, 1,2,3-trimethoxybenzene 86 undergoes selective demethylation to remove the methyl group at the 1-position, yielding syringol 60. In some embodiments, operation 640 includes the following steps:
i) preparing a first solution comprising 1,2,3-trimethoxybenzene 86 and a solvent;
ii) adding a second solution comprising a demethylation reagent and the solvent to the first solution to form a reaction mixture;
iii) heating the reaction mixture at a reaction temperature above 0° C. for a period of time sufficient to convert the 1,2,3-trimethoxybenzene 86 to syringol 60; and
iv) isolating the syringol.

In some embodiments, the solvent is dimethylformamide, dichloromethane (DCM), chloroform, heptane, tetrahydrofuran, 2-methyltetrahydrofuran, diglyme, 1,4-dioxane, chlorobenzene, benzene, anisole, acetonitrile (MeCN), 2-dichloroethane (DCE), or a mixture thereof. In some embodiments, the solvent is dimethylformamide. In some embodiments, the solvent is dichloromethane. In some embodiments, the solvent is chloroform. In some embodiments, the solvent is heptane. In some embodiments, the solvent is tetrahydrofuran. In some embodiments, the solvent is 2-methyltetrahydrofuran. In some embodiments, the solvent is diglyme. In some embodiments, the solvent is 1,4-dioxane. In some embodiments, the solvent is dichloromethane or heptane.

In some embodiments, the demethylation reagent comprises a Lewis acid including, but not limited to, ZnCl₂, LiCl, AlCl₃, BCl₃, FeCl₃, or a combination thereof. In some embodiments, the demethylation reagent is ZnCl₂. In some embodiments, the demethylation reagent is LiCl. In some embodiments, the demethylation reagent is AlCl₃. In some embodiments, the demethylation reagent is BCl₃. In some embodiments, the demethylation reagent comprises a mixture of AlCl₃ and BCl₃.

In some embodiments, the demethylation reagent comprises a trifluoroboron ether complex. In some embodiments, the demethylation reagent is a trifluoroboron diethyl ether complex (BF₃·OEt₂). In some embodiments, the demethylation reaction is enhanced by a quaternary ammonium salt, such as tetra-n-butylammonium bromide or tetra-n-butylammonium iodide. In some embodiments, the demethylation reagent is a mixture of a BF₃·OEt₂ and tetra-n-butylammonium bromide.

In some embodiments, the demethylation reagent comprises a Grignard reagent having the following structure: RMgX, wherein R is C₁-C₆ alkyl or C₃-C₆ cycloalkyl, and X is halogen. In some embodiments, R is methyl, ethyl, isopropyl, butyl, tert-butyl, sec-butyl, and cyclopropyl. In some embodiments, X is Br, Cl, or I. In some embodiments, the demethylation reagent may include MeMgBr, MeMgCl, EtMgBr, EtMgCl, iPrMgCl, iPrMgBr, or mixtures thereof. In some embodiments, the demethylation reagent is MeMgBr or MeMgCl.

In some embodiments, the demethylation reagent is halosilane. In some embodiments, the demethylation reagent is iodotrimethylsilane (Me₃Sil) or chlorotrimethyl (Me₃SiCl).

In some embodiments, a molar ratio of the demethylation reagent to 1,2,3-trimethoxybenzene 86 ranges from 0.1:1.0 to 5.0:1.0. In some embodiments, molar ratio of the demethylation reagent to 1,2,3-trimethoxybenzene 86 ranges from 0.1:1.0 to 4.0:1.0. In some embodiments, molar ratio of the demethylation reagent to 1,2,3-trimethoxybenzene 86 ranges from 0.1:1.0 to 3.0:1.0. In some embodiments, molar ratio of the demethylation reagent to 1,2,3-trimethoxybenzene 86 ranges from 0.1:1.0 to 2.0:1.0. In some embodiments, molar ratio of the demethylation reagent to 1,2,3-trimethoxybenzene 86 ranges from 1.0:1.0 to 2.0:1.0. In some embodiments, a molar ratio of the demethylation reagent to 1,2,3-trimethoxybenzene 86 ranges from 1.1:1.0 to 1.5:1.0. In some embodiments, a molar ratio of the demethylation reagent to 1,2,3-trimethoxybenzene 86 is about 1.5:1.0. In some embodiments, a molar ratio of the demethylation reagent to 1,2,3-trimethoxybenzene 86 is about 0.1:1.0. In some embodiments, a molar ratio of the demethylation reagent to 1,2,3-trimethoxybenzene 86 is about 0.2:1.0. In some embodiments, a molar ratio of the demethylation reagent to 1,2,3-trimethoxybenzene 86 is about 0.3:1.0. In some embodiments, a molar ratio of the demethylation reagent to 1,2,3-trimethoxybenzene 86 is about 0.4:1.0. In some embodiments, a molar ratio of the demethylation reagent to 1,2,3-trimethoxybenzene 86 is about 0.5:1.0. In some embodiments, a molar ratio of the demethylation reagent to 1,2,3-trimethoxybenzene 86 is about 0.6:1.0. In some embodiments, a molar ratio of the demethylation reagent to 1,2,3-trimethoxybenzene 86 is about 0.7:1.0. In some embodiments, a molar ratio of the demethylation reagent to 1,2,3-trimethoxybenzene 86 is about 0.8:1.0. In some embodiments, a molar ratio of the demethylation reagent to 1,2,3-trimethoxybenzene 86 is about 0.9:1.0. In some embodiments, a molar ratio of the demethylation reagent to 1,2,3-trimethoxybenzene 86 is about 1.0:1.0.

In some embodiments, a concentration of the demethylation reagent ranges from 1M to 5M. In some embodiments, a concentration of the demethylation reagent ranges from 1M to 3M. In some more specific embodiments, a concentration of the demethylation reagent is about 3M. In some embodiments, the reaction temperature is above 0° C. In some embodiments, the reaction temperature is above 5° C. In some embodiments, the reaction temperature is above 10° C. In some embodiments, the reaction temperature is above 15° C. In some embodiments, the reaction temperature is above 20° C. In some embodiments, the reaction temperature is above 25° C. In some embodiments, the reaction temperature is above 30° C. In some embodiments, the reaction temperature ranges from 5° C. to 20° C. In some embodiments, the reaction temperature ranges from 10° C. to 20° C. In some embodiments, the reaction temperature ranges from 10° C. to 30° C. In some embodiments, the reaction temperature ranges from 10° C. to 40° C. In some embodiments, the reaction temperature ranges from 10° C. to 50° C. In some embodiments, the reaction temperature ranges from 40° C. to 50° C. In some more specific embodiments, the reaction temperature is about 45° C.

In some embodiments, the second solution is added to the first solution over a period of time ranging from 5 to 60 minutes. In some embodiments, the second solution is added to the first solution over a period of time ranging from 5 to 50 minutes. In some embodiments, the second solution is added to the first solution over a period of time ranging from 5 to 40 minutes. In some embodiments, the second solution is added to the first solution over a period of time ranging from 5 to 30 minutes. In some embodiments, the second solution is added to the first solution over a period of time ranging from 5 to 20 minutes. In some embodiments, the second solution is added to the first solution over a period of time ranging from 10 to 20 minutes. In some embodiments, the second solution is added to the first solution over a period of time ranging from 10 to 30 minutes. In some embodiments, the second solution is added to the first solution over a period of time ranging from 10 to 40 minutes. In some embodiments, the second solution is added to the first solution over a period of time ranging from 10 to 50 minutes. In some embodiments, the second solution is added to the first solution over a period of time ranging from 10 to 60 minutes. In some embodiments, the second solution is added to the first solution over 1 hour.

In some embodiments, the second solution is added to the first solution by a dropwise manner or in portions. In some embodiments, the second solution is added to the first solution in three or more portions. In some embodiments, the second solution is added to the first solution in ten or more portions. In some embodiments, the second solution is added to the first solution in three or more portions over 5 to 60 minutes. In some embodiments, the second solution is added to the first solution in three or more portions over 5 to 50 minutes. In some embodiments, the second solution is added to the first solution in three or more portions over 5 to 40 minutes. In some embodiments, the second solution is added to the first solution in three or more portions over 5 to 30 minutes. In some embodiments, the second solution is added to the first solution in ten or more portions over 5 to 60 minutes. In some embodiments, the second solution is added to the first solution in ten or more portions over 5 to 50 minutes. In some embodiments, the second solution is added to the first solution in ten or more portions over 5 to 40 minutes. In some embodiments, the second solution is added to the first solution in ten or more portions over 5 to 30 minutes. In some embodiments, the second solution is added to the first solution in ten or more portions over 1 hour.

The crude syringol may then be purified using any of the techniques known in the field of organic chemistry, including liquid-liquid extraction, concentration (removal of solvent, by distillation for example), fractionation, precipitation, recrystallization, distillation, and chromatography. In some embodiments, the crude syringol may be purified by recrystallization in a mixed solvent of methyl tertiary butyl ether (MTBE) and heptane or a mixed solvent of DCM and heptane to afford syringol with a high purity over 99%. In some embodiments, after purification, the syringol can have a purity of at least 92%, for example, at least 93%, at least 94%, at least 95%, or at least 98%.

In some embodiments, the syringol 60 is obtained with over 91% yield. In some embodiments, the syringol 60 is obtained with over 80% yield. In some embodiments, the syringol 60 is obtained with over 90% yield. In some embodiments, the syringol 60 is obtained with over 95% yield. In some embodiments, the syringol 60 is obtained with over 96% yield. In some embodiments, the syringol 60 is obtained with over 97% yield. In some embodiments, the syringol 60 is obtained with over 98% yield. In some embodiments, the syringol 60 is obtained with over 99% yield.

Alternatively, instead of producing syringol 60 from gallic acid 82 derived from renewal tannic acid sources, gallic acid or pyrogallol can be obtained directly from commercial sources and used to produce syringol 60 according to Scheme 1.

Preparation Syringol from 4-Hydroxybenzoic Acid

Scheme 2 illustrates another exemplary synthetic route to obtain syringol 60 using 4-hydroxybenzoic acid 90 as a starting material. 4-Hydroxybenzoic acid 90 can be derived from renewal biomass or from commercial sources. In some embodiments, the biomass is lignocellulosic biomass, and 4-hydroxybenzoic acid 90 is obtained from lignin depolymerization described above according to methods 100-500.

According to Scheme 2, syringic acid can be synthesized from 4-hydroxybenzoic acid 90 via halogenation, followed by methoxylation and decarboxylation, to afford syringol 60.

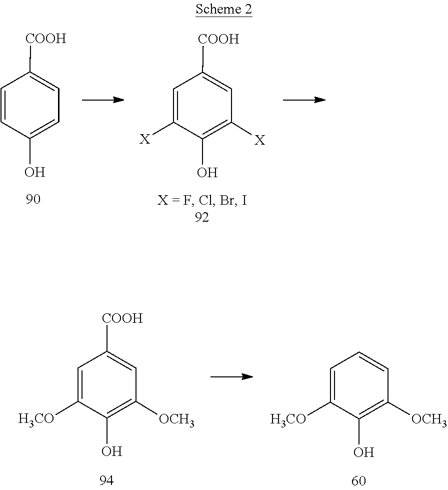

In one embodiment, a method for synthesizing syringol from 4-hydroxybenzoic acid is described. The method comprises halogenating 4-hydroxybenzoic acid 90 to form 3,5-dihalogenated 4-hydroxybenzoic acid 92; reacting the 3,5-dihalogenated 4-hydroxybenzoic acid 92 with an alkoxide to form 3,5-dimethoxy-4-hydroxybenzoic acid 94; and decarboxylating the 3,5-dimethoxy-4-hydroxybenzoic acid to form syringol 60.

In some embodiments, the 3,5-dihalogenated 4-hydroxybenzoic acid 92 is 3,5-dibromo 4-hydroxybenzoic acid or 3,5-dichloro 4-hydroxybenzoic acid. In some embodiments, the 3,5-dihalogenated 4-hydroxybenzoic acid 92 is 3,5-dibromo 4-hydroxybenzoic acid. In some embodiments, the 3,5-dihalogenated 4-hydroxybenzoic acid 92 is 3,5-difluoro 4-hydroxybenzoic acid. In some embodiments, the 3,5-dihalogenated 4-hydroxybenzoic acid 92 is 3,5-diiodo 4-hydroxybenzoic acid.

In some embodiments, the alkoxide is NaOCH₃. In some embodiments, the alkoxide is KOCH₃. In some embodiments, the alkoxide is LiOCH₃. In some embodiments, the alkoxide is KOCH₃,

In some embodiments, the 3,5-dihalogenated 4-hydroxybenzoic acid 92 is reacted with a metal halogen to form 2-hydroxyl 4,6-dimethoxy benzoic acid 94. The metal halogen is CuBr. In some embodiments, the metal halogen is Cul. In some embodiments, the metal halogen is CuCl. In some embodiments, the metal halogen is CuF.

In some embodiments, the 3,5-dibromo 4-hydroxybenzoic acid is reacted with NaOCH₃ and CuBr to form 2-hydroxy 5,3-dimethoxy benzoic acid 94. In some embodiments, the 3,5-dichloro 4-hydroxybenzoic acid is reacted with NaOCH₃ and CuBr to form 2-hydroxy 3,5-dimethoxy benzoic acid 94. In some embodiments, the 3,5-dibromo 4-hydroxybenzoic acid is reacted with a metal halogen at a temperature from 80° C. to 120° C. to form 2-hydroxy 3,5-dimethoxy benzoic acid 94. In some embodiments, the 3,5-dichloro 4-hydroxybenzoic acid is reacted with a metal halogen at a temperature from 80° C. to 120° C. to form 2-hydroxy 3,5-dimethoxy benzoic acid 94. The metal halogen is NaOCH₃ and CuBr.

In some embodiments, the 3,5-dibromo 4-hydroxybenzoic acid is reacted with NaOCH₃ and CuBr at a temperature from 80° C. to 120° C. to form 2-hydroxy 3,5-dimethoxy benzoic acid 94. In some embodiments, the 3,5-dichloro 4-hydroxybenzoic acid is reacted with NaOCH₃ and CuBr at a temperature from 80° C. to 120° C. to form 2-hydroxy 3,5-dimethoxy benzoic acid 94.

In some embodiments, the 2-hydroxy 3,5-dimethoxy benzoic acid 94 is decarboxylated with CuSO₄.

### Preparation of Polymerizable Syringol

Syringol 60 can react with (meth)acrylic anhydride or other reactive functional groups to form syringyl (meth)acrylate and other syringol-derived monomers to be used for 3D printing.

### Curable Resin Composition

The syringyl (meth)acrylate and other syringol-derived monomers according to the present disclosure can serve as reactive diluents to reduce the viscosity of viscous or highly viscous curable resins in 3D printing and can result in polymeric materials with favorable thermomechanical properties, such as stiffness and stress resistance that are suitable for use in orthodontic appliances, for example, for moving one or more teeth of a patient. The curable resin disclosed herein can be a photo-curable resin, a thermo-curable resin, or a combination thereof.

In some embodiments, the curable resin comprises 10-80 wt % of syringol-derived monomers, based on the total weight of the composition. In some embodiments, the curable resin comprises syringol-derived monomers at concentrations ranging from 15-45 wt % or 25-35 wt %, based on the total weight of the composition.

The curable resin of the present disclosure can comprise one or more photo-polymerizable components in addition to the syringol-derived monomers. Such one or more photo-polymerizable components can include one or more telechelic oligomers, one or more telechelic polymers, or a combination thereof. In such instances, a telechelic oligomer can have a number-average molecular weight of greater than 500 Da (0.5 kDa) but less than 5 kDa. A telechelic polymer can have a number-average molecular weight of greater than 10 kDa but less than 50 kDa. A telechelic polymer can have a number-average molecular weight of greater than 5 kDa but less than 50 kDa. A telechelic polymer can have a number-average molecular weight of greater than 5 kDa but less than 300 kDa. The telechelic oligomer(s) and/or polymer(s) can comprise photoreactive moieties at their termini. In some cases, the photoreactive moiety can be an acrylate, methacrylate, vinyl acrylate, vinyl methacrylate, allyl ether, silene, alkyne, alkene, vinyl ether, maleimide, fumarate, maleate, itoconate, or styrenyl moiety. In some cases, the photoreactive moiety can be an acrylate or a methacrylate. A telechelic polymer herein can include polyurethanes, polyesters, block copolymers or any other commercial polymers with reactive (e.g., photo-reactive) end groups. Thus, in various instances, a telechelic block copolymer of the present disclosure is capable of undergoing photopolymerization with one or more other telechelic polymers, telechelic block copolymers, telechelic oligomers, or syringol-derived monomers via its terminal monomers. In various cases, the terminal monomers comprise a photo-reactive moiety enabling further photo-polymerization reactions. Such photo-polymerization reaction of a telechelic block copolymer with other polymers, oligomers and/or monomers can occur during photo-curing, e.g., in instances where these components are part of a photo-curable resin. In some instances, a telechelic polymer can have one or more glass transition temperatures, wherein at least one glass transition temperature is at 0° C., or lower.

The curable resin disclosed herein can comprise about 10 to 70 wt %, 10 to 60 wt %, 10 to 50 wt %, 10 to 40 wt %, 10 to 30 wt %, 10 to 25 wt %, 20 to 60 wt %, 20 to 50 wt %, 20 to 40 wt %, 20 to 35 wt %, 20 to 30 wt %, 25 to 60 wt %, 25 to 50 wt %, 25 to 45 wt %, 25 to 40 wt %, or 25 to 35 wt %, of a telechelic polymer and/or oligomer, or any combination thereof, based on the total weight of the composition.

The curable resin described herein can further comprise an initiator that is a photoinitiator. Such photoinitiator, when activated with light of an appropriate wavelength (e.g., UV/VIS) can initiate a polymerization reaction (e.g., during photo-curing) between the telechelic polymers, syringol-derived monomers, and other potentially polymerizable components that may be present in the curable resin, to form a polymeric material as further described herein. Generally, photoinitiators described in the present disclosure can include those that can be activated with light and initiate polymerization of the polymerizable components of the formulation.

In some embodiments, the photoinitiator is a free radical photoinitiator. In certain embodiments, the free radical photoinitiator comprises an alpha hydroxy ketone moiety (e.g., 2-hydroxy-2-methylpropiophenone or 1-hydroxycyclohexyl phenyl ketone), an alpha-amino ketone (e.g., 2-benzyl-2-(dimethylamino)-4′-morpholinobutyrophenone or 2-methyl-1-[4-(methylthio)phenyl]-2-morpholinopropan-1-one), 4-methyl benzophenone, an azo compound (e.g., 4,4′-Azobis (4-cyanovaleric acid), 1,1′-Azobis (cyclohexanecarbonitrile), Azobisisobutyronitrile, 2,2′-Azobis (2-methylpropionitrile), or 2,2′-Azobis (2-methylpropionitrile)), an inorganic peroxide, an organic peroxide, or any combination thereof. In some embodiments, the composition comprises a photoinitiator comprising SpeedCure TPO-L (ethyl (2,4,6-trimethylbenzoyl)phenyl phosphinate). In some embodiments, the curable resin comprises a photoinitiator selected from a benzophenone, a mixture of benzophenone and a tertiary amine containing a carbonyl group which is directly bonded to at least one aromatic ring, and an Irgacure (e.g., Irgacure 907 (2-methyl-1-[4-(methylthio)-phenyl]-2-morpholino-propanone-1) or Irgacure 651 (2,2-dimethoxy-1,2-diphenylethan-1-one). In some embodiments, the photoinitiator comprises an acetophenone photoinitiator (e.g., 4′-hydroxyacetophenone, 4′-phenoxyacetophenone, 4′-ethoxyaceto-phenone), a benzoin, a benzoin derivative, a benzil, a benzil derivative, a benzophenone (e.g., 4-benzoylbiphenyl, 3,4-(dimethylamino)benzophenone, 2-methylbenzophenone), a cationic photoinitiator (e.g., diphenyliodonium nitrate, (4-iodophenyl)diphenylsulfonium triflate, triphenylsulfonium triflate), an anthraquinone, a quinone (e.g., camphorquinone), a phosphine oxide, a phosphinate, 9,10-phenanthrenequinone, a thioxanthone, any combination thereof, or any derivative thereof.

In some embodiments, the photoinitiator can have a maximum wavelength absorbance between 200 and 300 nm, between 300 and 400 nm, between 400 and 500 nm, between 500 and 600 nm, between 600 and 700 nm, between 700 and 800 nm, between 800 and 900 nm, between 150 and 200 nm, between 200 and 250 nm, between 250 and 300 nm, between 300 and 350 nm, between 350 and 400 nm, between 400 and 450 nm, between 450 and 500 nm, between 500 and 550 nm, between 550 and 600 nm, between 600 and 650 nm, between 650 and 700 nm, or between 700 and 750 nm. In some embodiments, the photoinitiator has a maximum wavelength absorbance between 300 to 500 nm.

In some embodiments, the curable resin of the present disclosure comprises more than one initiator (e.g., 2, 3, 4, 5, or more than 5 initiators). In some embodiments, the curable resin comprises an initiator that is a thermal initiator. In certain embodiments, the thermal initiator comprises an organic peroxide. In some embodiments, the thermal initiator comprises an azo compound, an inorganic peroxide, an organic peroxide, or any combination thereof. In some embodiments, the thermal initiator is selected from the group consisting of tert-amyl peroxybenzoate, 4,4-azobis (4-cyanovaleric acid), 1,1′-azobis(cyclohexanecarbonitrile), 2,2′-azobisisobutyronitrile (AIBN), benzoyl peroxide, 2,2-bis(tert-butylperoxy) butane, 1,1-bis(tert-butylperoxy)cyclohexane, 2,5-bis(tert-butylperoxy) 2,5-dimethylhexane, 2,5-bis(tert-butylperoxy)-2,5-dimethyl-3-hexyne, bis(1-(tert-butylperoxy)-3,3,5-trimethylcyclohexane, tert-butyl hydroxyperoxide, tert-butyl peracetate, tert-butyl peroxide, tert-butyl peroxybenzoate, tert-butylperoxy isopropyl carbonate, cumene hydroperoxide, cyclohexanone peroxide, dicumyl peroxide, lauroyl peroxide, 2,4-pentanedione peroxide, peracetic acid, potassium persulfate, a derivative thereof, and a combination thereof. In preferred embodiments, the thermal initiator comprises azobisisobutyronitrile, 2,2′-azodi(2-methylbutyronitrile), or a combination thereof.

In some embodiments, the curable resin comprises 0.01-10 wt %, 0.02-5 wt %, 0.05-4 wt %, 0.1-3 wt %, 0.1-2 wt %, or 0.1-1 wt % of the initiator, based on the total weight of the composition. In preferred embodiments, the curable resin comprises 0.1-2 wt % of the initiator. In some embodiments, the curable resin comprises 0.05 to 1 wt %, 0.05 to 2 wt %, 0.05 to 3 wt %, 0.05 to 4 wt %, 0.05 to 5 wt %, 0.1 to 1 wt %, 0.1 to 2 wt %, 0.1 to 3 wt %, 0.1 to 4 wt %, 0.1 to 5 wt %, 0.1 to 6 wt %, 0.1 to 7 wt %, 0.1 to 8 wt %, 0.1 to 9 wt %, or 0.1 to 10 wt % of the photoinitiator. In preferred embodiments, the curable resin comprises 0.1-2 wt % of the photoinitiator. In some embodiments, the curable resin comprises from 0 to 10 wt %, from 0 to 9 wt %, from 0 to 8 wt %, from 0 to 7 wt %, from 0 to 6 wt %, from 0 to 5 wt %, from 0 to 4 wt %, from 0 to 3 wt %, from 0 to 2 wt %, from 0 to 1 wt %, or from 0 to 0.5 wt % of the thermal initiator. In preferred embodiments, the curable resin comprises from 0 to 0.5 wt % of the thermal initiator.

In some embodiments, the curable resin of the present disclosure can comprise a crosslinking modifier (e.g., in addition to a polymerizable monomer that can act as a cross-linker, or in instances where the polymerizable monomer does not act as a cross-linker), a solvent, a glass transition temperature modifier, a toughness modifier, a polymerization catalyst, a polymerization inhibitor, a light blocker, a plasticizer, a surface energy modifier, a pigment, a dye, a filler, a biologically significant chemical, or a combination thereof.

In some aspects, the curable resin comprises a crosslinking modifier. A “crosslinking modifier” as used herein refers to a substance that bonds one oligomer or polymer chain to another oligomer or polymer chain, thereby forming a crosslink. A crosslinking modifier may become part of another substance, such as a crosslink in a polymer material obtained by a polymerization process. In some embodiments, a crosslinking modifier is a curable unit that, when mixed with the curable resin, is incorporated as a crosslink into the polymeric material that results from polymerization of the formulation. In certain embodiments, the curable resin comprises 0-25 wt % of the crosslinking modifier, based on the total weight of the composition. In some embodiments, the crosslinking modifier can have a number-average molecular weight equal to or less than 3 kDa, equal to or less than 2.5 kDa, equal to or less than 2 kDa, equal to or less than 1.5 kDa, equal to or less than 1.25 kDa, equal to or less than 1 kDa, equal to or less than 800 Da, equal to or less than 600 Da, or equal to or less than 400 Da. In some embodiments, the crosslinking modifier can have a high glass transition temperature (T_{g}), which leads to a high heat deflection temperature. In some embodiments, the crosslinking modifier has a glass transition temperature greater than −10° C., greater than −5° C., greater than 0° C., greater than 5° C., greater than 10° C., greater than 15° C., greater than 20° C., or greater than 25° C. In some embodiments, the crosslinking modifier comprises a (meth)acrylate-terminated polyester, a tricyclodecanediol di(meth)acrylate, a vinyl ester-terminated polyester, a tricyclodecanediol vinyl ester, a derivative thereof, or a combination thereof.

In some embodiments, the curable resin comprises a solvent. In some embodiments, the solvent comprises a nonpolar solvent. In certain embodiments, the nonpolar solvent comprises pentane, cyclopentane, hexane, cyclohexane, benzene, toluene, 1,4-dioxane, chloroform, diethyl ether, dichloromethane, a derivative thereof, or a combination thereof. In some embodiments, the solvent comprises a polar aprotic solvent. In certain embodiments, the polar aprotic solvent comprises tetrahydrofuran, ethyl acetate, acetone, dimethylformamide, acetonitrile, DMSO, propylene carbonate, a derivative thereof, or a combination thereof. In some embodiments, the solvent comprises a polar protic solvent. In certain embodiments, the polar protic solvent comprises formic acid, n-butanol, isopropyl alcohol, n-propanol, t-butanol, ethanol, methanol, acetic acid, water, a derivative thereof, or a combination thereof. In some embodiments, the curable resin comprises less than 90 wt % less than 80 wt %, less than 70 wt %, less than 60 wt %, less than 50 wt %, less than 40 wt %, less than 30 wt %, less than 20 wt %, less than 15 wt %, less than 10 wt %, less than 5 wt %, less than 3 wt %, less than 2 wt %, or less than 1 wt % of the solvent, based on the total weight of the composition. In some cases, the solvent is configured to evaporate or separate from the curable reins following curing.

In some embodiments, the curable resin herein comprises a component that can alter the glass transition temperature of the cured polymeric material. In such instances, a glass transition temperature modifier (also referred to herein as a T_{g} modifier or a glass transition modifier) can be present in a curable resin from about 0 to 50 wt %, based on the total weight of the composition. The T_{g} modifier can have a high glass transition temperature, which leads to a high heat deflection temperature, which can be necessary to use a material at elevated temperatures. In some embodiments, the curable resin comprises 0 to 80 wt %, 0 to 75 wt %, 0 to 70 wt %, 0 to 65 wt %, 0 to 60 wt %, 0 to 55 wt %, 0 to 50 wt %, 1 to 50 wt %, 2 to 50 wt %, 3 to 50 wt %, 4 to 50 wt %, 5 to 50 wt %, 10 to 50 wt %, 15 to 50 wt %, 20 to 50 wt %, 25 to 50 wt %, 30 to 50 wt %, 35 to 50 wt %, 0 to 40 wt %, 1 to 40 wt %, 2 to 40 wt %, 3 to 40 wt %, 4 to 40 wt %, 5 to 40 wt %, 10 to 40 wt %, 15 to 40 wt %, or 20 to 40 wt % of a T_{g} modifier, based on the total weight of the composition. In certain embodiments, the curable resin comprises 0-50 wt % of a glass transition modifier, based on the total weight of the composition. In some instances, the number average molecular weight of the T_{g} modifier is 0.4 to 5 kDa. In some embodiments, the number average molecular weight of the T_{g} modifier is from 0.1 to 5 kDa, from 0.2 to 5 kDa, from 0.3 to 5 kDa, from 0.4 to 5 kDa, from 0.5 to 5 kDa, from 0.6 to 5 kDa, from 0.7 to 5 kDa, from 0.8 to 5 kDa, from 0.9 to 5 kDa, from 1.0 to 5 kDa, from 0.1 to 4 kDa, from 0.2 to 4 kDa, from 0.3 to 4 kDa, from 0.4 to 4 kDa, from 0.5 to 4 kDa, from 0.6 to 4 kDa, from 0.7 to 4 kDa, from 0.8 to 4 kDa, from 0.9 to 4 kDa, from 1 to 4 kDa, from 0.1 to 3 kDa, from 0.2 to 3 kDa, from 0.3 to 3 kDa, from 0.4 to 3 kDa, from 0.5 to 3 kDa, from 0.6 to 3 kDa, from 0.7 to 3 kDa, from 0.8 to 3 kDa, from 0.9 to 3 kDa, or from 1 to 3 kDa.

In some embodiments, the curable resin herein comprises a toughness modifier that can alter the toughness of the cured polymeric material. In some cases, a toughness modifier may provide for high elongation at break and toughness via strengthening effects, and syringol-derived monomer described herein may improve the processability of the formulations, e.g., by acting as a reactive diluent, particularly of those compositions comprising high amounts of toughness modifiers, while maintaining high values for strength and T_{g}. The toughness modifier can have an average number average molecular weight from 2 to 20 kDa. The toughness modifier may comprise a polyolefin, a polyester, a polyurethane, a polyvinyl, a polyamide, a polyether, a polyacrylic acid, a polycarbonate, a polysulfone, a polyacrylate, a cellulose-based resin, a polyvinyl chloride, a polyvinylidene fluoride, a polyvinylidene chloride, a cycloolefin-based resin, a polybutadiene, a glycidyl methacrylate, or a methyl acrylic ester. For example, the toughness modifier may comprise a urethane group, a carbonate group, or both a urethane group and a carbonate group. In some embodiments, the toughness modifier includes a telechelic polymer. In some embodiments, the telechelic polymer is a polyol with (meth)acrylate end groups. In some embodiments, telechelic polyols include polyether diols, polyester diols, or polycarbonate diols. In some embodiments, the curable resin can comprise 10 to 70 wt %, 10 to 60 wt %, 10 to 50 wt %, 10 to 40 wt %, 10 to 30 wt %, 10 to 25 wt %, 20 to 60 wt %, 20 to 50 wt %, 20 to 40 wt %, 20 to 35 wt %, 20 to 30 wt %, 25 to 60 wt %, 25 to 50 wt %, 25 to 45 wt %, 25 to 40 wt %, or 25 to 35 wt %, of the toughness modifier, based on the total weight of the composition.

In some embodiments, the curable resin herein comprises a polymerization catalyst. In some embodiments, the polymerization catalyst comprises a tin catalyst, a platinum catalyst, a rhodium catalyst, a titanium catalyst, a silicon catalyst, a palladium catalyst, a metal triflate catalyst, a boron catalyst, a bismuth catalyst, or any combination thereof. Non-limiting examples of a titanium catalyst include di-n-butylbutoxychlorotin, di-n-butyldiacetoxytin, di-n-butyldilauryltin, dimethyldincodecanoatetin, dioctyldilauryltin, tetramethyltin, and dioctylbis (2-ethylhexylmaleate) tin. Non-limiting examples of a platinum catalyst include platinum-divinyltetramethyl-disiloxane complex, platinum-cyclovinylmethyl-siloxane complex, platinum-octanal complex, and platinum carbonyl cyclovinylmethylsiloxane complex. A non-limiting example of a rhodium catalyst includes tris (dibutylsulfide) rhodium trichloride. Non-limiting examples of a titanium catalyst includes titanium isopropoxide, titanium 2-ethyl-hexoxide, titanium chloride triisopropoxide, titanium ethoxide, and titanium diisopropoxide bis(ethylacetoacetate). Non-limiting examples of a silicon catalyst include tetramethylammonium siloxanolate and tetramethylsilylmethyl-trifluoromethane sulfonate. A non-limiting example of a palladium catalyst includes tetrakis (triphenylphosphine) palladium (0). Non-limiting examples of a metal triflate catalyst include scandium trifluoromethane sulfonate, lanthanum trifluoromethane sulfonate, and ytterbium trifluoromethane sulfonate. A non-limiting example of a boron catalyst includes tris (pentafluorophenyl) boron. Non-limiting examples of a bismuth catalyst include bismuth-zinc neodecanoate, bismuth 2-ethylhexanoate, a metal carboxylate of bismuth and zinc, and a metal carboxylate of bismuth and zirconium.

In some embodiments, the curable resin herein comprises a polymerization inhibitor in order to stabilize the composition and prevent premature polymerization. In some embodiments, the polymerization inhibitor is a photopolymerization inhibitor (e.g., oxygen). In some embodiments, the polymerization inhibitor is a phenolic compound (e.g., BHT). In some embodiments, the polymerization inhibitor is a stable radical (e.g., 2,2,4,4-tetramethylpiperidinyl-1-oxy radical, 2,2-diphenyl-1-picrylhydrazyl radical, galvinoxyl radical, or triphenylmethyl radical). In some embodiments, more than one polymerization inhibitor is present in the resin. In some embodiments, the polymerization inhibitor polymerization inhibitor is an antioxidant, a hindered amine light stabilizer (HAL), a hindered phenol, or a deactivated radical (e.g., a peroxy compound). In some embodiments, the polymerization inhibitor is selected from the group consisting of 4-tert-butylpyrocatechol, tert-butylhydroquinone, 1,4-benzoquinone, 6-tert-butyl-2,4-xylenol, 2-tertbutyl-1,4-benzoquinone, 2,6-di-tert-butyl-p-cresol, 2,6-ditert-butylphenol, 1,1-diphenyl-2-picrylhydrazyl free radical, hydroquinone, 4-methoxyphenol, phenothiazine, derivative thereof, and any combination thereof.

In some embodiments, the curable resin herein comprises a light blocker in order to dissipate UV radiation. In some embodiments, the light blocker absorbs a specific UV energy value and/or range. In some embodiments, the light blocker is a UV light absorber, a pigment, a color concentrate, or an IR light absorber. In some embodiments, the light blocker comprises a benzotriazole (e.g., 2-(2′-hydroxy-phenyl benzotriazole), 2,2-dihydroxy-4-methoxy benzophenone, 9,10-diethoxyanthracene, a hydroxyphenyl triazine, an oxanilide, a benzophenone, or a combination thereof. In some embodiments, the curable resin comprises from 0 to 10 wt %, from 0 to 9 wt %, from 0 to 8 wt %, from 0 to 7 wt %, from 0 to 6 wt %, from 0 to 5 wt %, from 0 to 4 wt %, from 0 to 3 wt %, from 0 to 2 wt %, from 0 to 1 wt %, or from 0 to 0.5 wt % of the light blocker, based on the total weight of the composition. In more specific embodiments, the curable resin comprises from 0 to 0.5 wt % of the light blocker.

In some embodiments, the curable resin herein comprises a filler. In some embodiments, the filler comprises calcium carbonate (i.e., chalk), kaolin, metakolinite, a kaolinite derivative, magnesium hydroxide (i.e., talc), calcium silicate (i.e., wollastonite), a glass filler (e.g., glass beads, short glass fibers, or long glass fibers), a nanofiller (e.g., nanoplates, nanofibers, or nanoparticles), a silica filler (e.g., a mica, silica gel, fumed silica, or precipitated silica), carbon black, dolomite, barium sulfate, Al(OH)₃, Mg(OH)₂, diatomaceous earth, magnetite, halloysite, zinc oxide, titanium dioxide, cellulose, lignin, a carbon filler (e.g., chopped carbon fiber or carbon fiber), a derivative thereof, or a combination thereof. The filler can be a minor constituent of the curable resin, for example accounting for less than 5 wt %, based on the total weight of the composition, or can account for a majority of the weight of the curable resin. In some embodiments, the filler is present as at least 0.05 wt %, at least 0.5 wt %, at least 1 wt %, at least 2 wt %, at least 3 wt %, at least 5 wt %, at least 8 wt %, at least 10 wt %, at least 12 wt %, at least 15 wt %, at least 20 wt %, at least 25 wt %, at least 30 wt %, at least 40 wt %, at least 50 wt %, at least 60 wt %, at least 70 wt %, at least 75 wt %, or at least 80 wt % of the curable resin. In some embodiments, the filler is present as at most 80 wt %, at most 75 wt %, at most 70 wt %, at most 60 wt %, at most 50 wt %, at most 40 wt %, at most 30 wt %, at most 25 wt %, at most 20 wt %, at most 15 wt %, at most 10 wt %, at most 8 wt %, at most 5 wt %, at most 3 wt %, at most 2 wt %, at most 1 wt %, or at most 0.5 wt % of the curable resin. In some embodiments, the filler is present between 0.05 and 60 wt %, between 1 and 5 wt %, between 1 and 10 wt %, between 1 and 20 wt %, between 2 and 5 wt %, between 2 and 10 wt %, between 2 and 20 wt %, between 3 and 6 wt %, between 3 and 10 wt %, between 3 and 20 wt %, between 5 and 10 wt %, between 5 and 25 wt %, between 8 and 20 wt %, between 10 and 60 wt %, between 12 and 25 wt %, between 15 and 30 wt %, between 15 and 40 wt %, between 20 and 35 wt %, between 25 and 50 wt %, between 30 and 50 wt %, between 35 and 65 wt %, between 40 and 65 wt %, between 40 and 80 wt %, between 50 and 75 wt %, or between 60 and 80 wt % of the curable resin. In some embodiments, the filler is present between 10 and 60 wt % of the curable resin. In some embodiments, the filler is present between 20 and 60 wt % of the curable resin. In some embodiments, the filler is present between 20 and 40 wt % of the curable resin. In some embodiments, the filler is present between 30 and 50 wt % of the curable resin.

In some embodiments, the curable resin herein comprises a pigment, a dye, or a combination thereof. A pigment is typically a suspended solid that may be insoluble in the resin. A dye is typically dissolved in the curable resin. In some embodiments, the pigment comprises an inorganic pigment. In some embodiments, the inorganic pigment comprises an iron oxide, barium sulfide, zinc oxide, antimony trioxide, a yellow iron oxide, a red iron oxide, ferric ammonium ferrocyanide, chrome yellow, carbon black, or aluminum flake. In some embodiments, the pigment comprises an organic pigment. In some embodiments, the organic pigment comprises an azo pigment, an anthraquinone pigment, a copper phthalocyanine (CPC) pigment (e.g., phthalo blue or phthalo green) or a combination thereof. In some embodiments, the dye comprises an azo dye (e.g., a diarylide or Sudan stain), an anthraquinone (e.g., Oil Blue A or Disperse Red 11), or a combination thereof. In some embodiments, the curable resin comprises from about 0.001 to about 3 wt % of the pigment. In some embodiments, the curable resin comprises from about 0.005 to about 2 wt % of the pigment, based on the total weight of the composition. In some cases, the curable resin comprises from about 0.005 to about 0.5 wt % of the pigment, based on the total weight of the composition. In some embodiments, the curable resin comprises from about 0.01 to about 0.3 wt % of the pigment, based on the total weight of the composition. In some embodiments, the curable resin comprises from about 0.005 to about 0.1 wt % of the pigment, based on the total weight of the composition.

In some embodiments, the curable resin herein comprises a surface energy modifier. In some embodiments, the surface energy modifier can aid the process of releasing a polymer from a mold. In some embodiments, the surface energy modifier can act as an antifoaming agent. In some embodiments, the surface energy modifier comprises a defoaming agent, a deaeration agent, a hydrophobization agent, a leveling agent, a wetting agent, or an agent to adjust the flow properties of the curable resin. In some embodiments, the surface energy modifier comprises an alkoxylated surfactant, a silicone surfactant, a sulfosuccinate, a fluorinated polyacrylate, a fluoropolymer, a silicone, a star-shaped polymer, an organomodified silicone, or any combination thereof. In some embodiments, the curable resin comprises from between about 0.01 to about 3 wt % of the surface energy modifier, based on the total weight of the composition. In some embodiments, the curable resin comprises from about 0.05 to about 1.5 wt %, from about 0.1 to about 1.5 wt %, from about 0.3 to about 1.5 wt %, from about 0.1 to about 1 wt %, from about 0.1 to about 0.5 wt %, from about 0.2 to about 1 wt %, from about 0.3 to about 0.7 wt %, or from about 0.4 to about 1 wt % of the surface energy modifier, based on the total weight of the composition.

In some embodiments, the curable resin herein comprises a plasticizer. A plasticizer can be a nonvolatile material that can reduce interactions between polymer chains, which can decrease glass transition temperature, melt viscosity, and elastic modulus. In some embodiments, the plasticizer comprises a dicarboxylic ester plasticizer, a tricarboxylic ester plasticizer, a trimellitate, an adipate, a sebacate, a maleate, or a bio-based plasticizer. In some embodiments, the plasticizer comprises a dicarboxylic ester or a tricarboxylic ester comprising a dibasic ester, a phthalate, bis(2-ethylhexyl) phthalate (DEHP), bis(2-propylheptyl) phthalate (DPHP), diisononyl phthalate (DINP), di-n-butyl phthalate (DBP), butyl benzyl phthalate (BBZP), diisodecyl phthalate (DIDP), dioctyl phthalate (DOP), diisooctyl phthalate (DIOP), diethyl phthalate (DEP), diisobutyl phthalate (DIBP), di-n-hexyl phthalate, a derivative thereof, or a combination thereof. In some embodiments, the plasticizer comprises a trimellitate comprising trimethyl trimellitate (TMTM), tri-(2-ethylhexyl)trimellitate (TEHTM), tri-(n-octyl, n-decyl)trimellitate (ATM), tri (heptyl, nonyl)trimellitate (LTM), n-octyl trimellitate (OTM), trioctyl trimellitate, a derivative thereof, or a combination thereof. In some embodiments, the plasticizer comprises an adipate comprising bis(2-ethylhexyl) adipate (DEHA), dimethyl adipate (DMAD), monomethyl adipate (MMAD), dioctyl adipate (DOA), Bis [2-(2-butoxyethoxy)ethyl] adipate, dibutyl adipate, diisobutyl adipate, diisodecyl adipate, a derivative thereof, or a combination thereof. In some embodiments, the plasticizer comprises a sebacate comprising dibutyl sebacate (DBS), Bis (2-ethylhexyl) sebacate, diethyl sebacate, dimethyl sebacate, a derivative thereof, or a combination thereof. In some embodiments, the plasticizer comprises a maleate comprises Bis (2-ethyl-hexyl) maleate, dibutyl maleate, diisobutyl maleate, a derivative thereof, or a combination thereof. In some embodiments, the plasticizer comprises a bio-based plasticizer comprising an acetylated monoglyceride, an alkylcitrate, a methyl ricinoleate, or a green plasticizer. In some embodiments, the alkyl citrate is selected from the group consisting of triethyl citrate, acetyl triethyl citrate, tributyl citrate, acetyl tributyl citrate, trioctyl citrate, acetyl trioctyl citrate, trihexyl citrate, acetyl trihexyl citrate, butyryl trihexyl citrate, trimethyl citrate, a derivative thereof, or a combination thereof. In some embodiments, the green plasticizer is selected from the group consisting of epoxidized soybean oil, epoxidized vegetable oil, epoxidized esters of soybean oil, a derivative thereof, or a combination thereof. In some embodiments, the plasticizer comprises an azelate, a benzoate (e.g., sucrose benzoate), a terephthalate (e.g., dioctyl terephthalate), 1, 2-cyclohexane dicarbonxylic acid diisononyl ester, alkyl sulphonic acid phenyl ester, a sulfonamide (e.g., N-ethyl toluene sulfonamide, N-(2-hydroxy propyl)benzene sulfonamide, N-(n-butyl)benzene sulfonamaide), an organophosphate (e.g., tricresyl phosphate or tributyl phosphate), a glycol (e.g., tricthylene glycol dihexanoate or tetraethylene glycol diheptanoate), a polyether, polybutene, a derivative thereof, or a combination thereof.

In some embodiments, the curable resin herein comprises a biologically significant chemical. In some embodiments, the biologically significant chemical comprises a hormone, an enzyme, an active pharmaceutical ingredient, an antibody, a protein, a drug, or any combination thereof. In some embodiments, the biologically significant chemical comprises a pharmaceutical composition, a chemical, a gene, a polypeptide, an enzyme, a biomarker, a dye, a compliance indicator, an antibiotic, an analgesic, a medical grade drug, a chemical agent, a bioactive agent, an antibacterial, an antibiotic, an anti-inflammatory agent, an immune-suppressive agent, an immune-stimulatory agent, a dentinal desensitizer, an odor masking agent, an immune reagent, an anesthetic, a nutritional agent, an antioxidant, a lipopolysaccharide complexing agent or a peroxide.

In some embodiments, the added component (e.g., a crosslinking modifier, a glass transition temperature modifier, a toughness modifier, a polymerization catalyst, a polymerization inhibitor, a light blocker, a plasticizer, a solvent, a surface energy modifier, a pigment, a dye, a filler, or a biologically significant chemical) is functionalized so that it can be incorporated into the polymeric material so that it cannot readily be extracted from the final cured material. In certain embodiments, the polymerization catalyst, polymerization inhibitor, light blocker, plasticizer, surface energy modifier, pigment, dye, and/or filler, are functionalized to facilitate their incorporation into the cured polymeric material.

### Resin Properties

The curable resin disclosed herein can be characterized by having one or more properties. In some embodiments, the syringol-derived monomer according to the present disclosure can be used as a reactive diluent in curable resins disclosed herein. Hence, in some instances, the syringol-derived monomer can reduce the viscosity of the curable resin (e.g., a photo-curable resin). In such cases, the syringol-derived monomer can reduce the viscosity of the curable resin by at least about 5% compared to a resin that does not comprise the polymerizable monomer. In some instances, the syringol-derived monomer can reduce the viscosity of the curable resin by at least about 5%, 10%, 20%, 30%, 40%, or 50%. In some instances, the curable resin of this disclosure can have a viscosity from about 30 cP to about 50,000 cP at a printing temperature. In some embodiments, the curable resin has a viscosity less than or equal to 30,000 cP, less than or equal to 25,000 cP, less than or equal to 20,000 cP, less than or equal to 19,000 cP, less than or equal to 18,000 cP, less than or equal to 17,000 cP, less than or equal to 16,000 cP, less than or equal to 15,000 cP, less than or equal to 14,000 cP, less than or equal to 13,000 cP, less than or equal to 12,000 cP, less than or equal to 11,000 cP, less than or equal to 10,000 cP, less than or equal to 9,000 cP, less than or equal to 8,000 cP, less than or equal to 7,000 cP, less than or equal to 6,000 cP, or less than or equal to 5,000 cP at 25° C. In some embodiments, the curable resin has a viscosity less than 15,000 cP at 25° C. In some embodiments, the curable resin has a viscosity less than or equal to 100,000 cP, less than or equal to 90,000 cP, less than or equal to 80,000 cP, less than or equal to 70,000 cP, less than or equal to 60,000 cP, less than or equal to 50,000 cP, less than or equal to 40,000 cP, less than or equal to 35,000 cP, less than or equal to 30,000 cP, less than or equal to 25,000 cP, less than or equal to 20,000 cP, less than or equal to 15,000 cP, less than or equal to 10,000 cP, less than or equal to 5,000 cP, less than or equal to 4,000 cP, less than or equal to 3,000 cP, less than or equal to 2,000 cP, less than or equal to 1,000 cP, less than or equal to 750 cP, less than or equal to 500 cP, less than or equal to 250 cP, less than or equal to 100 cP, less than or equal to 90 cP, less than or equal to 80 cP, less than or equal to 70 cP, less than or equal to 60 cP, less than or equal to 50 cP, less than or equal to 40 cP, less than or equal to 30 cP, less than or equal to 20 cP, or less than or equal to 10 cP at a printing temperature. In some embodiments, the curable resin has a viscosity from 50,000 cP to 30 cP, from 40,000 cP to 30 cP, from 30,000 cP to 30 cP, from 20,000 cP to 30 cP, from 10,000 cP to 30 cP, or from 5,000 cP to 30 cP at a printing temperature. In some embodiments, the printing temperature is from 0° C. to 25° C., from 25° C. to 40° C., from 40° C. to 100° C., or from 20° C. to 150° C. In some embodiments, the curable resin has a viscosity from 30 cP to 50,000 cP at a printing temperature, wherein the printing temperature is from 20° C. to 150° C. In yet other embodiments, the curable resin has a viscosity less than 20,000 cP at a print temperature. In some embodiments, the print temperature is from 10° C. to 200° C., from 15° C. to 175° C., from 20° C. to 150° C., from 25° C. to 125° C., or from 30° C. to 100° C. In preferred embodiments, the print temperature is from 20° C. to 150° C.

The curable resin of the present disclosure can be capable of being 3D printed at a temperature greater than 25° C. In some embodiments, the printing temperature is at least about 30° C., 40° C., 50° C., 60° C., 80° C., or 100° C. As described herein, the syringol-derived monomer of the present disclosure can have a low vapor pressure and/or mass loss at the printing temperature, thereby providing improved printing conditions compared to conventional resins used in additive manufacturing.

In some embodiments, the curable resin herein has a melting temperature greater than room temperature. In some embodiments, the curable resin has a melting temperature greater than 20° C., greater than 25° C., greater than 30° C., greater than 35° C., greater than 40° C., greater than 45° C. greater than 50° C., greater than 55° C., greater than 60° C., greater than 65° C., greater than 70° C., greater than 75° C., or greater than 80° C. In some embodiments, the curable resin has a melting temperature from 20° C. to 250° C., from 30° C. to 180° C., from 40° C. to 160° C., or from 50° C. to 140° C. In some embodiments, the curable resin has a melting temperature greater than 60° C. In other embodiments, the curable resin has a melting temperature from 80° C. to 110° C. In some instances, the curable resin can have a melting temperature of about 80° C. before polymerization, and after polymerization, the resulting polymeric material can have a melting temperature of about 100° C.

In certain instances, it may be advantageous that the curable resin is in a liquid phase at an elevated temperature. As an example, a conventional curable resin can comprise polymerizable components that may be viscous at a process temperature, and thus can be difficult to use in the fabrication of objects (e.g., using 3D printing). As a solution for that technical problem, the present disclosure provides curable resins comprising syringol-derived monomers that have a decreased viscosity at the elevated temperature, which can make such resin more applicable and usable for uses such as 3D printing. Hence, in some embodiments, provided herein are curable resins that are a liquid at an elevated temperature. In some embodiments, the elevated temperature is at or above the melting temperature (Tm) of the curable resin. In certain embodiments, the elevated temperature is a temperature in the range from about 40° C. to about 100° C., from about 60° C. to about 100° C., from about 80° C. to about 100° C., from about 40° C. to about 150° C., or from about 150° C. to about 350° C. In some embodiments, the elevated temperature is a temperature above about 40° C., above about 60° C., above about 80° C., or above about 100° C. In some embodiments, the curable resin disclosed herein is a liquid at an elevated temperature with a viscosity less than about 50 Pa's, less than about 20 Pa's, less than about 10 Pas, less than about 5 Pas, or less than about 1 Pa·s. In some embodiments, the curable resin disclosed herein is a liquid at an elevated temperature of above about 40° C. with a viscosity less than about 20 Pa·s. In yet other embodiments, the curable resin disclosed herein is a liquid at an elevated temperature of above about 40° C. with a viscosity less than about 1 Pa s.

In some embodiments, at least a portion of the curable resin disclosed herein has a melting temperature below about 100° C., below about 90° C., below about 80° C., below about 70° C., or below about 60° C. In some embodiments, at least a portion of the curable resin disclosed herein melts at an elevated temperature between about 100° C. and about 20° C., between about 90° C. and about 20° C., between about 80° C. and about 20° C., between about 70° C. and about 20° C., between about 60° C. and about 20° C., between about 60° C. and about 10° C., or between about 60° C. and about 0° C.

In various embodiments, the curable resin herein as well as its photo-polymerizable components can be biocompatible, bioinert, or a combination thereof. In various instances, the photo-polymerizable monomers of a resin herein can have biocompatible and/or bioinert metabolic (e.g., hydrolysis) products.

The curable resin of the present disclosure can comprise less than about 20 wt % or less than about 10 wt % hydrogen bonding units. In some embodiments, the curable resin herein comprises less than about 15 wt %, less than about 10 wt %, less than about 9 wt %, less than about 8 wt %, less than about 7 wt %, less than about 6 wt %, less than about 5 wt %, less than about 4 wt %, less than about 3 wt %, less than about 2 wt %, or less than about 1 wt % hydrogen bonding units, wherein wt % is the weight percent of species, including monomeric units in polymerized, oligomerized, and monomeric form, capable of forming at least one hydrogen bond.

### Polymeric Materials

The present disclosure provides polymeric materials formed from the curable resins disclosed herein. In preferred embodiments, the polymeric materials are formed from the curable resins via additive manufacturing.

In some embodiments, the polymeric material disclosed herein is characterized by one or more of: a tensile modulus greater than or equal to 200 MPa after 24 hours in a wet environment at 37° C.; a flexural stress of greater than or equal to 1.5 MPa remaining after 24 hours in a wet environment at 37° C.; a hardness from 60 Shore A to 85 Shore D after 24 hours in a wet environment at 37° C.; an elongation at break greater than or equal to 15% before 24 hours in a wet environment at 37° C.; and an elongation at break greater than or equal to 15% after 24 hours in a wet environment at 37° C.

Property values of the polymeric material can be determined, for example, by using the following methods:
stress relaxation properties can be assessed using an RSA-G2 instrument from TA Instruments, with a 3-point bending, according to ASTM D790; stress relaxation can be measured at 30° C. and submerged in water, and reported as either the remaining load after 24 hours, and/or as the percent (%) of initial load;
storage modulus can be measured at 37° C. and is reported in MPa;
T_{g} of the cured polymeric material can be assessed using dynamic mechanical analysis (DMA) and is provided herein as the tan δ peak when run at 1 Hz with a temperature ramp of 2° C. a minute;
tensile modulus, tensile strength, elongation at yield and elongation at break can be assessed according to ISO 527-2 5B;
tensile strength at yield, elongation at break, tensile strength, and Young's modulus can be assessed according to ASTM D1708 or ASTM D638; and
flexural stress remaining after 24 hours in wet environment at 37° C. (“flexural stress remaining”) can be assessed according to ASTM E328.

In some embodiments, the polymeric material is characterized by a tensile stress-strain curve that displays a yield point after which the test specimen continues to elongate, but there is no increase in load. Such yield point behavior typically occurs “near” the glass transition temperature, where the material is between the glassy and rubbery regimes and may be characterized as having viscoelastic behavior. In embodiments, viscoelastic behavior is observed in the temperature range 20° C. to 40° C. The yield stress is determined at the yield point. In some embodiments, the yield point follows an elastic region in which the slope of the stress-strain curve is constant or nearly constant. In embodiments, the modulus is determined from the initial slope of the stress-strain curve or as the secant modulus at 1% strain (e.g., when there is no linear portion of the stress-strain curve). The elongation at yield is determined from the strain at the yield point. When the yield point occurs at a maximum in the stress, the ultimate tensile strength is less than the yield strength. For a tensile test specimen, the strain is defined by In (l/l₀), which may be approximated by (l-l₀)/l₀ at small strains (e.g., less than approximately 10%) and the elongation is l/l₀, where 1 is the gauge length after some deformation has occurred and l₀ is the initial gauge length. The mechanical properties can depend on the temperature at which they are measured. The test temperature may be below the expected use temperature for a dental appliance such as 35° C. to 40° C. In some embodiments, the test temperature is 23±2° C.

Properties of the polymeric materials can be determined after a soak time in a wet environment. Determination of values after a soak time in a wet environment can be conducted on a 1-mm thick sample. For example, material properties of a polymeric material disclosed herein can be determined by obtaining a 1-mm thick sample of said polymeric material, and soaking in a wet environment for 24 hours at 37° C. (i.e., the material after 24 hours in a wet environment at 37° C.).

In some embodiments, the polymeric material has an elongation at break greater than 5%, greater than 10%, greater than 15%, greater than 20%, greater than 25%, greater than 30%, greater than 35%, greater than 40%, greater than 45%, or greater than 50% after 24 hours in a wet environment at 37° C. In some embodiments, the polymeric material has an elongation at break greater than 5%, greater than 10%, greater than 15%, greater than 20%, greater than 25%, greater than 30%, greater than 35%, greater than 40%, greater than 45%, or greater than 50% before 24 hours in a wet environment at 37° C. In preferred embodiments, the polymeric material has an elongation at break greater than 15% before and after 24 hours in a wet environment at 37° C.

In some embodiments, the polymeric material has a tensile strength at yield from 1 MPa to 100 MPa, from 5 MPa to 85 MPa, from 10 MPa to 75 MPa, from 15 MPa to 65 MPa, from 20 MPa to 55 MPa, or from 25 MPa to 45 MPa after 24 hours in a wet environment at 37° C. In preferred embodiments, the polymeric material has a tensile strength at yield from 10 MPa to 55 MPa after 24 hours in a wet environment at 37° C. In some embodiments, the polymeric material is characterized by a tensile strength at yield greater than or equal to 0.1 MPa, greater than or equal to 0.5 MPa, greater than or equal to 1 MPa, greater than or equal to 2 MPa, greater than or equal to 3 MPa, greater than or equal to 4 MPa, greater than or equal to 5 MPa, greater than or equal to 6 MPa, greater than or equal to 7 MPa, greater than or equal to 8 MPa, greater than or equal to 9 MPa, or greater than or equal to 10 MPa after 24 hours in a wet environment at 37° C. In preferred embodiments, the polymeric material is characterized by a tensile strength at yield greater than or equal to 5 MPa after 24 hours in a wet environment at 37° C.

In some embodiments, the polymeric material has an ultimate tensile strength from 10 MPa to 100 MPa, from 15 MPa to 80 MPa, from 20 MPa to 60 MPa, from 25 MPa to 50 MPa, from 25 MPa to 45 MPa, from 25 MPa to 40 MPa, from 30 MPa to 45 MPa, or from 30 MPa to 40 MPa after 24 hours in a wet environment at 37° C. In preferred embodiments, the polymeric material has an ultimate tensile strength from 10 MPa to 50 MPa after 24 hours in a wet environment at 37° C.

In some embodiments, the polymeric material has a tensile modulus from 100 MPa to 3000 MPa, from 200 MPa to 3000 MPa, from 250 MPa to 2750 MPa, from 400 MPa to 2500 MPa, from 600 MPa to 2250 MPa, or from 800 MPa to 2000 MPa after 24 hours in a wet environment at 37° C. In some embodiments, the polymeric material has a tensile modulus of greater or equal to 100 MPa, greater or equal to 200 MPa, greater or equal to 300 MPa, greater or equal to 400 MPa, greater or equal to 500 MPa, greater or equal to 600 MPa, greater or equal to 700 MPa, greater or equal to 800 MPa, greater or equal to 900 MPa, greater or equal to 1000 MPa, greater or equal to 1100 MPa, greater or equal to 1200 MPa, greater or equal to 1300 MPa, greater or equal to 1400 MPa, or greater or equal to 1500 MPa after 24 hours in a wet environment at 37° C. In some preferred embodiments, the polymeric material has a tensile modulus of greater than 200 MPa after 24 hours in a wet environment at 37° C. In some preferred embodiments, the polymeric material has a tensile modulus from 1.0 GPa to 1.4 GPa after 24 hours in a wet environment at 37° C. In some preferred embodiments, the polymeric material is characterized by a tensile modulus greater than or equal to 200 MPa after 24 hours in a wet environment at 37° C.

In some embodiments, the polymeric material has a flexural stress relaxation remaining (“flexural stress remaining”) greater than or equal to 5%, greater than or equal to 10%, greater than or equal to 15%, greater than or equal to 20%, greater than or equal to 35%, greater than or equal to 40%, greater than or equal to 45%, greater than or equal to 50%, greater than or equal to 55%, or greater than or equal to 60% after 24 hours in a wet environment at 37° C. In some preferred embodiments the polymeric material has a flexural stress remaining of greater than or equal to 10% after 24 hours in a wet environment at 37° C. In some embodiments, the polymeric material is characterized by a flexural stress remaining from 5% to 45%, from 10% to 45%, from 15% to 45%, from 20% to 45%, from 25% to 45%, or from 30% to 45% of the initial load after 24 hours in a wet environment at 37° C. In some preferred embodiments, the polymeric material is characterized by a flexural stress remaining from 20% to 45% of the initial load after 24 hours in a wet environment at 37° C.

In some embodiments, the polymeric material is characterized by a flexural stress remaining from 0.01 MPa to 15 MPa, from 0.05 MPa to 15 MPa, from 0.1 MPa to 15 MPa, from 0.5 MPa to 15 MPa, from 1 MPa to 15 MPa, from 2 MPa to 15 MPa, from 3 MPa to 15 MPa, from 4 MPa to 15 MPa, from 5 MPa to 15 MPa, or from 10 MPa to 15 MPa after 24 hours in a wet environment at 37° C. In some preferred embodiments, the polymeric material is characterized by a flexural stress remaining from 2 MPa to 15 MPa after 24 hours in a wet environment at 37° C. In some embodiments, the polymeric material is characterized by a flexural stress of greater than or equal to 0.1 MPa, greater than or equal to 0.5 MPa, greater than or equal to 1 MPa, greater than or equal to 1.5 MPa, greater than or equal to 2 MPa, greater than or equal to 2.5 MPa, greater than or equal to 3 MPa, greater than or equal to 4 MPa, greater than or equal to 5 MPa, greater than or equal to 6 MPa, greater than or equal to 7 MPa, greater than or equal to 8 MPa, greater than or equal to 9 MPa, greater than or equal to 10 MPa, or greater than or equal to 15 MPa remaining after 24 hours in a wet environment at 37° C. In some preferred embodiments, the polymeric material is characterized by a flexural stress of greater than or equal to 1.5 MPa remaining after 24 hours in a wet environment at 37° C.

In certain embodiments, it is advantageous that the polymeric materials have a high flexural stress remaining, forming relatively stiff materials. In some applications relating to use of hard materials (e.g., aeronautical engineering, medical implants), a polymeric material formed from the curable resins disclosed herein would be advantageous due to the availability of conventional 3D printers to form these polymeric materials having the desired characteristics. In some embodiments, the polymeric material has a flexural modulus remaining of 50 MPa or more, 60 MPa or more, 70 MPa or more, 80 MPa or more, 90 MPa or more, 100 MPa or more, 125 MPa or more, or 150 MPa or more after 24 hours in a wet environment at 37° C.

In certain other embodiments, it is advantageous that the polymeric materials have a relatively low flexural stress remaining, forming materials that are not overly-stiff. In some embodiments, the polymeric material has a flexural modulus remaining of 80 MPa or less, 70 MPa or less, 60 MPa or less, 55 MPa or less, 50 MPa or less, or 45 MPa or less after 24 hours in a wet environment at 37° C. In some embodiments, the flexural modulus is measured under static conditions at a loading rate of 32 mm/min and a strain of 5%.

In some embodiments, a polymeric material will have a flexural stress remaining after a period of time of use. As a non-limiting example, an orthodontic appliance (e.g., an aligner) can be formed of a polymeric material having a high flexural stress. In some embodiments, following application of the appliance to the teeth of a patient, there can be a significant and fast decrease of flexural stress (e.g., over the course of minutes). Such decreases in flexural stress can follow an exponential curve of decrease leading towards an asymptote during the intended lifetime of the appliance (e.g., over the course of weeks for an orthodontic appliance such as an aligner). Orthodontic appliances may have an initial period of discomfort that, following a period of use, decreases corresponding with a decrease of flexural stress remaining. In some embodiments, the polymeric material has a flexural stress remaining of 90 MPa or less, 85 MPa or less, 80 MPa or less, 75 MPa or less, 70 MPa or less, 65 MPa or less, 60 MPa or less, 55 MPa or less, or 50 MPa or less after a time period of use. In preferred embodiments, the polymeric material has a flexural stress remaining of 80 MPa or less after a time period of use. In some embodiments, the time period of use is 1 minute, 5 minutes, 10 minutes, 30 minutes, 1 hour, or 24 hours. As a non-limiting example, an aligner composed of polymeric material placed onto a patient's teeth that is removed after 10 minutes and has a flexural stress of 70 MPa would have a polymeric material characterized by a flexural stress remaining of 70 MPa after a time period of use, wherein said time period is 10 minutes.

In certain embodiments, the polymeric material is characterized by an elongation at yield of greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 9%, greater than or equal to 10%, greater than or equal to 15%, or greater than or equal to 20% after 24 hours in a wet environment at 37° C. (i.e., following exposure to a wet environment for 24 hours). In some embodiments, the polymeric material is characterized by an elongation at yield of greater than or equal to 1%, greater than or equal to 2%, greater than or equal to 3%, greater than or equal to 4%, greater than or equal to 5%, greater than or equal to 6%, greater than or equal to 7%, greater than or equal to 8%, greater than or equal to 9%, greater than or equal to 10%, greater than or equal to 15%, or greater than or equal to 20% before 24 hours in a wet environment at 37° C. In preferred embodiments, the polymeric material is characterized by an elongation at yield of greater than or equal to 4% before and after 24 hours in a wet environment at 37° C.

In some embodiments, the polymeric material has a maximum force at 5% strain greater than or equal to 3 pound-force per 3.57 cm². As a non-limiting example, the sample is a rectangular slab having a width of 2.1 cm and a distance between support of 1.7 cm. In some embodiments, the polymeric material has a remaining force greater than 0.1 pound-force per 3.57 cm² after being submerged for 24 hours in a wet environment having a temperature of 37° C.

In some embodiments, the polymeric material is characterized by an elongation at yield from 1% to 10%, from 2% to 10%, from 3% to 10%, from 4% to 10%, from 5% to 10%, from 1% to 15%, from 1% to 20%, or from 1% to 25% after 24 hours in a wet environment at 37° C. In some embodiments, the polymeric material is characterized by an elongation at yield from 1% to 10%, from 2% to 10%, from 3% to 10%, from 4% to 10%, from 5% to 10%, from 1% to 15%, from 1% to 20%, or from 1% to 25% before 24 hours in a wet environment at 37° C. In preferred embodiments, the polymeric material is characterized by an elongation at yield from 4% to 10% before and after 24 hours in a wet environment at 37° C.

In some embodiments, the polymeric material is characterized by an elongation at break of greater than 5%, greater than 10%, greater than 15%, greater than 20%, greater than 25%, greater than 30%, greater than 35%, greater than 40%, greater than 45%, or greater than 50% after 24 hours in a wet environment at 37° C. In some embodiments, the polymeric material is characterized by an elongation at break of greater than 5%, greater than 10%, greater than 15%, greater than 20%, greater than 25%, greater than 30%, greater than 35%, greater than 40%, greater than 45%, or greater than 50% before 24 hours in a wet environment at 37° C. In preferred embodiments, the polymeric material is characterized by an elongation at break of greater than 15% before and after 24 hours in a wet environment at 37° C. In some embodiments, the polymeric material is characterized by an elongation at break from 5% to 250%, from 10% to 250%, from 15% to 250%, from 20% to 250%, from 25% to 250%, from 30% to 250%, from 35% to 250%, from 40% to 250%, from 45% to 250%, from 50% to 250%, from 75% to 250%, or from 100% to 250% after 24 hours in a wet environment at 37° C. In some embodiments, the polymeric material is characterized by an elongation at break from 5% to 250%, from 10% to 250%, from 15% to 250%, from 20% to 250%, from 25% to 250%, from 30% to 250%, from 35% to 250%, from 40% to 250%, from 45% to 250%, from 50% to 250%, from 75% to 250%, or from 100% to 250% before 24 hours in a wet environment at 37° C. In preferred embodiments, the polymeric material is characterized by an elongation at break from 40% to 250% before and after 24 hours in a wet environment at 37° C.

In some embodiments, the polymeric material is characterized by a storage modulus from 0.1 MPa to 4000 MPa, from 50 MPa to 2750 MPa, from 100 MPa to 2500 MPa, from 200 MPa to 2250 MPa, from 300 MPa to 3000 MPa, from 500 MPa to 3000 MPa, from 750 MPa to 3000 MPa, or from 1000 MPa to 3000 MPa after 24 hours in a wet environment at 37° C. In preferred embodiments, the polymeric material is characterized by a storage modulus from 750 MPa to 3000 MPa after 24 hours in a wet environment at 37° C.

In some embodiments, the polymeric material has at least one glass transition temperature (T_{g}) from 0° C. to 150° C. In preferred embodiments, the polymeric material has at least one glass transition temperature greater than 60° C. In even more preferred embodiments, the polymeric material has at least one glass transition temperature greater than 75° C. In some embodiments, the at least one glass transition temperature is from 0° C. to 200° C., from 0° C. to 140° C., from 0° C. to 20° C., from 20° C. to 40° C., from 40° C. to 60° C., from 60° C. to 80° C., from 80° C. to 100° C., from 100° C. to 120° C., from 120° C. to 140° C., from 140° C. to 160° C., from 160° C. to 180° C., from 180° C. to 200° C., from 0° C. to 35° C., from 35° C. to 65° C., from 65° C. to 100° C., from 0° C. to 50° C., or from 50° C. to 100° C. In some embodiments, the polymeric material has at least one glass transition temperature from 0° C. to 10° C., from 10° C. to 20° C., from 20° C. to 30° C., from 30° C. to 40° C., from 40° C. to 50° C., from 50° C. to 60° C., from 60° C. to 70° C., from 70° C. to 80° C., or from 80° C. to 90° C. In some embodiments, the polymeric material has at least one glass transition temperature from −100° C. to 40° C., from −80° C. to 10° C., from −70° C. to 0° C., from −70° C. to −10° C., from −70° C. to −20° C., from −70° C. to −30° C., from −70° C. to −40° C., from −70° C. to −50° C., or from −80° C. to −40° C. In some embodiments, the polymeric material has at least two glass transition temperatures. In certain embodiments, the polymeric material has a first glass transition temperature less than or equal to 40° C. and a second glass transition temperature greater than or equal to 60° C. In some embodiments, the polymeric material has a first glass transition temperature less than or equal to 0° C. and a second glass transition temperature greater than or equal to 60° C. In some embodiments, the polymeric material has a first glass transition temperature less than or equal to 0° C. and a second glass transition temperature greater than or equal to 75° C. In some embodiments, the polymeric material has a first glass transition temperature less than −20° C. and a second glass transition temperature greater than 80° C.

Low levels of water absorption are favorable for polymeric materials described herein. Water absorption can occur when a polymeric material is exposed to a wet environment (e.g., a patient's mouth using an orthodontic appliance formed from a polymeric material). Properties of the polymeric material can degrade when water absorption reaches a threshold value, typically greater than 22 wt %. It is preferred herein that the polymeric materials have low levels of water uptake. In some embodiments, the polymeric material formed from the curable resin comprises a water uptake of less than 25 wt %, less than 20 wt %, less than 15 wt %, less than 10 wt %, less than 5 wt %, less than 4 wt %, less than 3 wt %, less than 2 wt %, less than 1 wt %, less than 0.5 wt %, less than 0.25 wt %, or less than 0.1 wt %. In preferred embodiments, the polymeric material formed from the curable resin comprises a water uptake of less than 2 wt %. In more preferred embodiments, the polymeric material formed from the curable resin comprises a water uptake of less than 1 wt %. In even more preferred embodiments, the polymeric material formed from the curable resin comprises a water uptake of less than 0.5 wt %. In embodiments described herein, the water uptake is measured after 24 hours in a wet environment at 37° C. In some embodiments, a polymer formed from the oligomer of the resin is hydrophobic. In preferred embodiments, the polymeric material formed from the curable resin is hydrophobic.

In some embodiments, the polymeric material is characterized by having a low water uptake. In some embodiments, the polymeric material comprises less than 40 wt %, less than 35 wt %, less than 30 wt %, less than 25 wt %, less than 20 wt %, less than 15 wt %, less than 10 wt %, less than 5 wt %, less than 4 wt %, less than 3 wt %, less than 2 wt %, or less than 1 wt % water after 24 hours in a wet environment at 37° C. In preferred embodiments, the polymeric material comprises less than 2 wt % water after 24 hours in a wet environment at 37° C. In more preferred embodiments, the polymeric material comprises less than 1 wt % water after 24 hours in a wet environment at 37° C. In even more preferred embodiments, the polymeric material comprises less than 0.5 wt % water after 24 hours in a wet environment at 37° C.

The polymeric materials formed from the curable resins can have high conversion rates, or high extent of reactions. In some embodiments, the polymeric material comprises 20-100 wt % of the polymer formed from the oligomer(s) and/or monomer(s) of the curable resin. In some embodiments, the polymeric material comprises greater than 20 wt %, greater than 30 wt %, greater than 40 wt %, greater than 50 wt %, greater than 60 wt %, greater than 70 wt %, greater than 80 wt %, greater than 85 wt %, greater than 90 wt %, greater than 95 wt %, greater than 98 wt %, or greater than 99 wt % of the polymer formed from the oligomer(s) and/or monomer(s) of the curable resin. In certain embodiments, it is preferable to have a high conversion percentage of oligomer(s) and/or monomer(s) into polymer when forming the polymeric material. In some embodiments, the formed polymer is hydrophobic.

In some embodiments, the polymeric materials formed from the curable resins have high conversion rates of reactive group double bonds (e.g., acrylates or methacrylates) to single bonds, indicating incorporation into the polymeric material. In some embodiments, the conversion of reactive double bonds in the resin to single bonds in the polymeric material can be measured by FTIR (e.g., by measuring relative amounts before and after curing). In some embodiments, the polymeric material formed from the curable resin has greater than 60%, greater than 70%, greater than 80%, greater than 90%, or greater than 95% conversion of double bonds to single bonds.

In some embodiments, the polymeric materials formed from the curable resins have low levels of extractable materials (e.g., unreacted monomers from said curable resin). The amount of extractable materials can be determined by weight loss of the polymeric material after soaking in water for 1 week, after soaking in ethanol for 48 hours, or after soaking in hexane for 48 hours. A general experiment for determining the amount of extractable material includes the steps of (i) weighing a dried sample of the polymeric material; (ii) soaking the sample in a solvent at a given temperature (e.g., 25° C.) for a period of time; (iii) refreshing the solvent until extraction is completed; (iv) drying the sample in an oven; (v) weighing the extracted sample; and (vi) calculating the weight loss. In some embodiments, the polymeric materials formed from the curable resin have less than 5 wt %, less than 4 wt %, less than 3 wt %, less than 2 wt %, less than 1 wt %, less than 0.75 wt %, less than 0.5 wt %, or less than 0.25 wt % extractable materials.

In some embodiments, the polymeric material has less than 20 wt %, less than 15 wt %, less than 10 wt %, less than 9 wt %, less than 8 wt %, less than 7 wt %, less than 6 wt %, less than 5 wt %, less than 4 wt %, less than 3 wt %, less than 2 wt %, or less than 1 wt % hydrogen bonding units, as calculated or measured by weight percentage of hydrogen bonding groups, further described herein. In preferred embodiments, the polymeric material has less than or equal to 10 wt % hydrogen bonding units.

In some cases, the polymeric material comprises a color defined by an L* value between 70 and 95, an a* value of between −11 and 1, and a b* value of between 2 and 22. In some cases, the polymeric material comprises a color defined by an L* value between 78 and 87, an a* value of between −7 and −3, and a b* value of between 8 and 16.

In some embodiments, the polymeric material is clear, substantially clear, mostly clear, or opaque. In certain embodiments, the polymeric material is clear. In certain embodiments, the polymeric material is substantially clear. In certain embodiments, the polymeric material is mostly clear. In some embodiments, greater than 70% of visible light passes through the polymeric material. In certain embodiments, greater than 80% of visible light passes through the polymeric material. In certain embodiments, greater than 90% of visible light passes through the polymeric material. In certain embodiments, greater than 95% of visible light passes through the polymeric material. In certain embodiments, greater than 99% of visible light passes through the polymeric material. Transparency can be measured using a UV-Vis spectrophotometer. In some embodiments, the transparency is measured by measuring the passage of a wavelength of transparency. In some embodiments, greater than 70%, greater than 80%, greater than 90%, greater than 95%, or greater than 99% of the wavelength of transparency can pass through the polymeric material. In some embodiments, the wavelength of transparency is in the visible light range (i.e., from 400 nm to 800 nm), is in the infrared light range, or is in the ultraviolet light range. In some embodiments, the polymeric material does not have color. In other embodiments, the polymeric material appears white, off-white, or mostly transparent with white coloring, as detected by the human eye.

In some embodiments, greater than 20%, greater than 30%, greater than 40%, greater than 50%, greater than 60%, greater than 70%, greater than 80%, greater than 90%, or greater than 95% of visible light passes through the polymeric material after 24 hours in a wet environment at 37° C. In preferred embodiments, greater than 70% of visible light passes through the polymeric material after 24 hours in a wet environment at 37° C.

In some embodiments, the polymeric material is biocompatible, bioinert, or a combination thereof.

In some embodiments, the polymeric material is formed using 3D printing (i.e., by additive manufacturing) using photopolymerization. In certain embodiments, the polymeric material is formed using conventional 3D printers. In some embodiments, the polymeric material can be used in coatings, molds, injection molding machines, or other manufacturing methods that use or could use light during the curing process. In some embodiments, the polymeric material is well suited for applications that require, e.g., solvent resistance, humidity resistance, water resistance, creep resistance, or heat deflection resistance.

### Methods of Producing Polymeric Materials

Further provided herein is a method of polymerizing (e.g., photo-curing) a curable resin (e.g., a photo-curable resin) comprising a syringol-derived monomer and one or more additional components selected from the group consisting of telechelic polymers, telechelic oligomers, polymerization initiators, polymerization inhibitors, solvents, fillers, antioxidants, pigments, colorants, surface modifiers, and mixtures thereof, to obtain a crosslinked polymer, the method comprising a step of mixing the resin components, optionally after heating, with a syringol-derived monomer before inducing polymerization by heating and/or irradiating the composition.

The present disclosure provides methods for producing polymeric materials using curable resins described herein. In various embodiments, provided herein are methods for photo-curing photo-curable resins. Hence, in various instances, provided herein is a method of forming a polymeric material, the method comprising: (i) providing a photo-curable resin of the present disclosure; (ii) exposing the photo-curable resin to a light source; and curing the photo-curable resin to form the polymeric material.

In some embodiments, the photo-curing comprises a single curing step. In some embodiments, the photo-curing comprises a plurality of curing steps. In yet other embodiments, the photo-curing comprises at least one curing step which exposes the curable resin to light. Exposing the curable resin to light can initiate and/or facilitate photo-polymerization. In some instances, a photoinitiator can be used as part of the resin to accelerate and/or initiate photo-polymerization. In some embodiments, the resin is exposed to UV (ultraviolet) light, visible light, IR (infrared) light, or any combination thereof. In some embodiments, the cured polymeric material is formed from the photo-curable resin using at least one step comprising exposure to a light source, wherein the light source comprises UV light, visible light, and/or IR light. In some embodiments, the light source comprises a wavelength from 10 nm to 200 nm, from 200 nm to 350 nm, from 350 nm to 450 nm, from 450 nm to 550 nm, from 550 nm to 650 nm, from 650 nm to 750 nm, from 750 nm to 850 nm, from 850 nm to 1000 nm, or from 1000 nm to 1500 nm.

In some instances, the polymeric material has the glass transition temperature (T_{g}) of at least about 40° C., 50° C., 60° C., 80° C., 90° C., 100° C., 110° C. or at least about 120° C.

In some embodiments, a method of forming a polymeric material from a curable resin described herein can further comprise initiating and/or enhancing formation of crystalline phases in the forming polymeric material. In certain embodiments, the triggering comprises cooling the cured material, adding seeding particles to the resin, providing a force to the cured material, providing an electrical charge to the resin, or any combination thereof. In some cases, polymer crystals can yield upon application of a strain (e.g., a physical strain, such as twisting or stretching a material). The yielding may include unraveling, unwinding, disentangling, dislocation, coarse slips, and/or fine slips in the crystallized polymer. In some embodiments, the methods disclosed herein further comprise the step of growing polymer crystals. As described further herein, polymer crystals comprise the crystallizable polymeric material.

Thus, in various embodiments, a method of forming a polymeric material from a curable resin described herein can comprise inducing phase separation in the forming polymeric material (i.e., during photo-curing), wherein such phase separation can yield polymeric materials that comprise one or more amorphous phases, one or more crystalline phases, or both one or more amorphous phases and one or more crystalline phases.

As described herein, a polymeric material produced by the methods provided herein can be characterized by one or more of: (i) a storage modulus greater than or equal to 200 MPa; (ii) a flexural stress of greater than or equal to 1.5 MPa remaining after 24 hours in a wet environment at 37° C.; (iii) an elongation at break greater than or equal to 5% before and after 24 hours in a wet environment at 37° C.; (iv) a water uptake of less than 25 wt % when measured after 24 hours in a wet environment at 37° C.; and (v) transmission of at least 30% of visible light through the polymeric material after 24 hours in a wet environment at 37° C. In various cases, such polymeric material can be characterized by at least 2, 3, 4, or all of these properties.

### Fabrication and Use of 3D Printed Objects

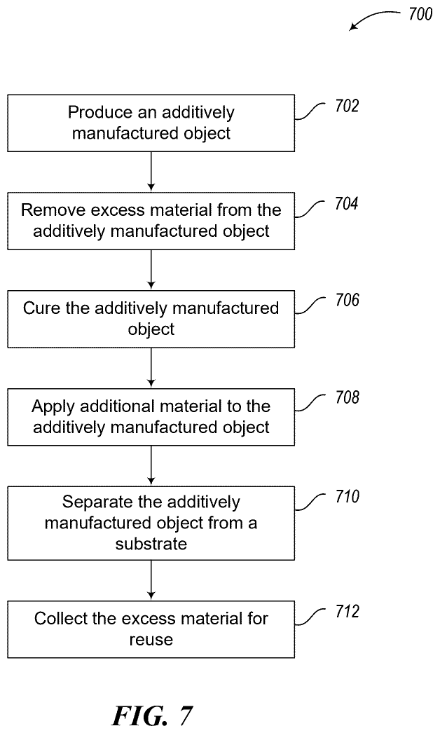

FIG. 7 is a flow diagram providing a general overview of a method 700 for fabricating and post-processing an additively manufactured object, in accordance with embodiments of the present disclosure. The method 700 can be used to produce many different types of additively manufactured objects, such as orthodontic appliances (e.g., aligners, palatal expanders, attachments, attachment templates, retainers), restorative objects (e.g., crowns, veneers, implants), and/or other dental appliances (e.g., oral sleep apnea appliances, mouth guards). Additional examples of orthodontic appliances and associated methods that are applicable to the present disclosure are described below.

The method 700 begins at operation 702 with producing an additively manufactured object. The additively manufactured object can be produced using any suitable additive manufacturing technique known to those of skill in the art. Additive manufacturing (also referred to herein as “3D printing”) includes a variety of technologies which fabricate 3D objects directly from digital models through an additive process. In some embodiments, additive manufacturing includes depositing a precursor material (e.g., a photo-curable resin) onto a build platform. The precursor material can be cured, polymerized, melted, sintered, fused, and/or otherwise solidified to form a portion of the object and/or combine the portion with previously formed portions of the object. In some embodiments, the additive manufacturing techniques provided herein build up the object geometry in a layer-by-layer fashion, with successive layers being formed in discrete build steps. Alternatively, or in combination, the additive manufacturing techniques described herein can allow for continuous build-up of an object geometry.

Examples of additive manufacturing techniques suitable for use with the methods described herein include, but are not limited to, the following: (1) vat photopolymerization, in which an object is constructed from a vat of liquid photopolymer resin, including techniques such as stercolithography (SLA), digital light processing (DLP), continuous liquid interface production (CLIP), two-photon induced photopolymerization (TPIP), and volumetric additive manufacturing; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer-by-layer, and direct ink writing (DIW); (5) powder bed fusion, including techniques such as direct metal laser sintering (DMLS), electron beam melting (EBM), selective heat sintering (SHS), selective laser melting (SLM), and selective laser sintering (SLS); (6) sheet lamination, including techniques such as laminated object manufacturing (LOM) and ultrasonic additive manufacturing (UAM); and (7) directed energy deposition, including techniques such as laser engineering net shaping, directed light fabrication, direct metal deposition, and 3D laser cladding.

For example, the additively manufactured object can be fabricated using a vat photopolymerization process in which light is used to selectively cure a vat or reservoir of a curable material (e.g., a polymeric resin). Each layer of curable material can be selectively exposed to light in a single exposure (e.g., DLP) or by scanning a beam of light across the layer (e.g., SLA). Vat polymerization can be performed in a “top-down” or “bottom-up” approach, depending on the relative locations of the vat, light source, and build platform.

As another example, the additively manufactured object can be fabricated using high temperature lithography (also known as “hot lithography”). High temperature lithography can include any photopolymerization process that involves heating a photopolymerizable material (e.g., a polymeric resin). For example, high temperature lithography can involve heating the material to a temperature of at least 30° C., 40° C., 50° C., 60° C., 70° C., 80° C., 90° C., 100° C., 110° C., or 120° C. In some embodiments, the material is heated to a temperature within a range from 50° C. to 120° C., from 90° C. to 120° C., from 100° C. to 120° C., from 105° C. to 115° C., or from 108° C. or 110° C. The heating can lower the viscosity of the photopolymerizable material before and/or during curing. Accordingly, high temperature lithography can be used to fabricate objects from highly viscous and/or poorly flowable materials, which, when cured, may exhibit improved mechanical properties (e.g., stiffness, strength, stability) compared to other types of materials. For example, high temperature lithography can be used to fabricate objects from a material having a viscosity of at least 5 Pas, 10 Pas, 15 Pas, 20 Pas, 30 Pas, 40 Pas, or 50 Pa's at 20° C. Representative examples of high-temperature lithography processes that may be incorporated in the methods herein are described in International Publication Nos. WO 2015/075094, WO 2016/078838, WO 2018/032022, WO 2020/070639, WO 2021/130657, and WO 2021/130661, the disclosures of each of which are incorporated herein by reference in their entirety.

In a further example, the additively manufactured object can be fabricated using a selective laser sintering process involving using a laser beam to selectively melt and fuse a layer of powdered material according to a desired cross-sectional shape to build up the object geometry. As another example, the additively manufactured object can be fabricated using a fused deposition modeling process involving melting and selectively depositing a thin filament of thermoplastic polymer in a layer-by-layer manner to form an object. In yet another example, the additively manufactured object can be fabricated using a material jetting process involving jetting or extruding one or more materials onto a build surface to form successive layers of the object geometry.

In some embodiments, the additively manufactured object is fabricated using continuous liquid interphase production (also known as “continuous liquid interphase printing”) in which the object is continuously built up from a reservoir of photo-curable resin by forming a gradient of partially cured resin between the building surface of the object and a polymerization-inhibited “dead zone.” In some embodiments, a semi-permeable membrane is used to control transport of a photopolymerization inhibitor (e.g., oxygen) into the dead zone to form the polymerization gradient. Representative examples of continuous liquid interphase production processes that may be incorporated in the methods herein are described in U.S. Patent Publication Nos. 2015/0097315, 2015/0097316, and 2015/0102532, the disclosures of each of which are incorporated herein by reference in their entirety.

As another example, a continuous additive manufacturing method can achieve continuous build-up of an object geometry by continuous movement of the build platform (e.g., along the vertical or Z-direction) during the irradiation phase, such that the hardening depth of the irradiated photopolymer is controlled by the movement speed. Accordingly, continuous polymerization of material on the build surface can be achieved. Such methods are described in U.S. Pat. No. 7,892,474, the disclosure of which is incorporated herein by reference in its entirety. In another example, a continuous additive manufacturing method can involve extruding a composite material composed of a curable liquid material surrounding a solid strand. The composite material can be extruded along a continuous three-dimensional path to form the object. Such methods are described in U.S. Patent Publication No. 2014/0061974, the disclosure of which is incorporated herein by reference in its entirety. In yet another example, a continuous additive manufacturing method can utilize a “heliolithography” approach in which the liquid photopolymer is cured with focused radiation while the build platform is continuously rotated and raised. Accordingly, the object geometry can be continuously built up along a spiral build path. Such methods are described in U.S. Patent Publication No. 2014/0265034, the disclosure of which is incorporated herein by reference in its entirety.

In a further example, the additively manufactured object can be fabricated using a volumetric additive manufacturing (VAM) process in which an entire object is produced from a 3D volume of resin in a single print step, without requiring layer-by-layer build up. During a VAM process, the entire build volume is irradiated with energy, but the projection patterns are configured such that only certain voxels will accumulate a sufficient energy dosage to be cured. Representative examples of VAM processes that may be incorporated into the present disclosure include tomographic volumetric printing, holographic volumetric printing, multiphoton volumetric printing, and xolography. For instance, a tomographic VAM process can be performed by projecting 2D optical patterns into a rotating volume of photosensitive material at perpendicular and/or angular incidences to produce a cured 3D structure. A holographic VAM process can be performed by projecting overlapping light patterns into a stationary reservoir of photosensitive material. A xolography process can use photoswitchable photoinitiators to induce local polymerization inside a volume of photosensitive material upon linear excitation by intersecting light beams of different wavelengths. Additional details of VAM processes suitable for use with the present disclosure are described in U.S. Pat. No. 11,370,173, U.S. Patent Publication No. 2021/0146619, U.S. Patent Publication No. 2022/0227051, International Publication No. WO 2017/115076, International Publication No. WO 2020/245456, International Publication No. WO 2022/011456, and U.S. Provisional Patent Application No. 63/181,645, the disclosures of each of which are incorporated herein by reference in their entirety.

The additively manufactured object can be made of any suitable material or combination of materials. As discussed above, in some embodiments, the additively manufactured object is made partially or entirely out of a polymeric material, such as a curable polymeric resin. The resin can be composed of one or more monomer components that are initially in a liquid state. The resin can be in the liquid at room temperature (e.g., 20° C.) or at an elevated temperature (e.g., a temperature within a range from 50° C. to 120° C.). When exposed to energy (e.g., light), the monomer components can undergo a polymerization reaction such that the resin solidifies into the desired object geometry. Representative examples of curable polymeric resins and other materials suitable for use with the additive manufacturing techniques herein are described in International Publication Nos. WO 2019/006409, WO 2020/070639, and WO 2021/087061, the disclosures of each of which are incorporated herein by reference in their entirety.

Optionally, the additively manufactured object can be fabricated from a plurality of different materials (e.g., at least two, three, four, five, or more different materials). The materials can differ from each other with respect to composition, curing conditions (e.g., curing energy wavelength), material properties before curing (e.g., viscosity), material properties after cured (e.g., stiffness, strength, transparency), and so on. In some embodiments, the additively manufactured object is formed from multiple materials in a single manufacturing step. For instance, a multi-tip extrusion apparatus can be used to selectively dispense multiple types of materials from distinct material supply sources to fabricate an object from a plurality of different materials. Examples of such methods are described in U.S. Pat. Nos. 6,749,414 and 11,318,667, the disclosures of which are incorporated herein by reference in their entirety. Alternatively, or in combination, the additively manufactured object can be formed from multiple materials in a plurality of sequential manufacturing steps. For instance, a first portion of the object can be formed from a first material in accordance with any of the methods herein, then a second portion of the object can be formed from a second material in accordance with methods herein, and so on, until the entirety of the object has been formed.

After the additively manufactured object is fabricated, the object can undergo one or more additional process steps, also referred to herein as “post-processing.” As described in detail below with respect to operations 704-712, post-processing can include removing excess material from the object, applying additional material(s) to the object, performing additional curing, separating the object from any supports or other structures that are not intended to be present in the final product, and/or collecting the removed excess material for reuse.

For example, at operation 704, the method 700 continues with removing excess material from the additively manufactured object. The excess material can include uncured material (e.g., unpolymerized liquid resin) and/or other unwanted material (e.g., debris) that remains on the additively manufactured object after fabrication. For example, certain materials used in additive manufacturing (e.g., highly viscous polymeric resins used in high temperature lithography) may adhere to the surface of the additively manufactured object. Additionally, excess material may accumulate on or within certain object features, such as cavities, crevices, indentations, apertures, etc. Accordingly, the additively manufactured object may need to be cleaned before further processing and use.

The excess material can be removed in many ways. In some embodiments, for example, the excess material is removed by rotating the additively manufactured object to centrifugally separate the excess material from the surfaces of the object. The rotation can be performed using a suitable device or system (e.g., a centrifuge) including components for supporting and applying rotational force to the additively manufactured object. Alternatively or in combination, the excess material can be removed by spraying or otherwise applying fluids (e.g., water, solvents) to the object, partially or fully immersing the object in a fluid, blowing a gas (e.g., air) on the object, applying a vacuum to the object, applying other types of mechanical forces to the object (e.g., vibration, agitation, tumbling, brushing), and/or other cleaning techniques known to those of skill in the art.

At operation 706, the method 700 can optionally include curing the additively manufactured object. This additional curing step (also known as “post-curing”) can be used in situations where the additively manufactured object is still in a partially cured “green” state after fabrication. For example, the curing energy used to fabricate the additively manufactured object at operation 702 may only partially polymerize the resin forming the object. Accordingly, the post-curing step may be needed to fully cure (e.g., fully polymerize) the additively manufactured object to its final, usable state. Post-curing can provide various benefits, such as improving the mechanical properties (e.g., stiffness, strength) and/or temperature stability of the additively manufactured object. Post-curing can be performed by heating the object, applying radiation (e.g., ultraviolet (UV), visible, microwave) to the object, or suitable combinations thereof. Post-curing can be performed by a specialized device (e.g., an oven or curing station) or can be performed by the same device used to rotate the additively manufactured object at operation 704. In other embodiments, however, the post-curing process of operation 706 is optional and can be omitted.

At operation 708, the method 700 can optionally include applying an additional material to the additively manufactured object. For example, the additional material can be a coating, such as a polymeric coating. The coating can be applied to one or more surfaces of the object for various purposes, including, but not limited to: providing a smooth surface finish, which can be beneficial for aesthetics and/or to improve user comfort if the object is intended to be in contact with the user's body (e.g., an orthodontic appliance worn on the teeth); coloring and/or applying other aesthetic features to the object; improving scratch resistance and/or other mechanical properties; providing antimicrobial properties; and incorporating therapeutic agents into the object for controlled release.

At operation 710, the method 700 can include separating the additively manufactured object from a substrate. In some embodiments, the substrate is a build platform which mechanically supports the object during fabrication and the post-processing steps described herein. The additively manufactured object can be connected to the substrate via a sacrificial region of cured material. Accordingly, the additively manufactured object can be detached from the substrate by applying pressure to fracture the sacrificial region. Once separated, the additively manufactured object can then be prepared for packaging, shipment, and use.

At operation 512, the method 700 can optionally include collecting the excess material removed from the additively manufactured object at operation 704. The excess material can include uncured material that is still suitable for reuse in subsequent additive manufacturing processes (e.g., the fabrication process of operation 702). Accordingly, operation 512 can include collecting the excess material (e.g., via containers, absorbent elements, piping, etc.) and, optionally, separating reusable excess material from other unwanted components that may be present (e.g., water, solvents, debris) via filtration, distillation, centrifugation, and/or other suitable techniques.

The method 700 can be modified in many ways. For example, although the above steps of the method 700 are described with respect to a single additively manufactured object, the method 700 can be used to concurrently fabricate and post-process any suitable number of additively manufactured objects, such as tens, hundreds, or thousands of additively manufactured objects. As another example, the ordering of the steps shown in FIG. 7 can be varied, e.g., the material application process of operation 708 can be performed before the curing process of operation 706. Some of the steps of the method 700 can be omitted, such as any of operations 706, 708, and/or 512. The method 700 can also include additional steps not shown in FIG. 7.

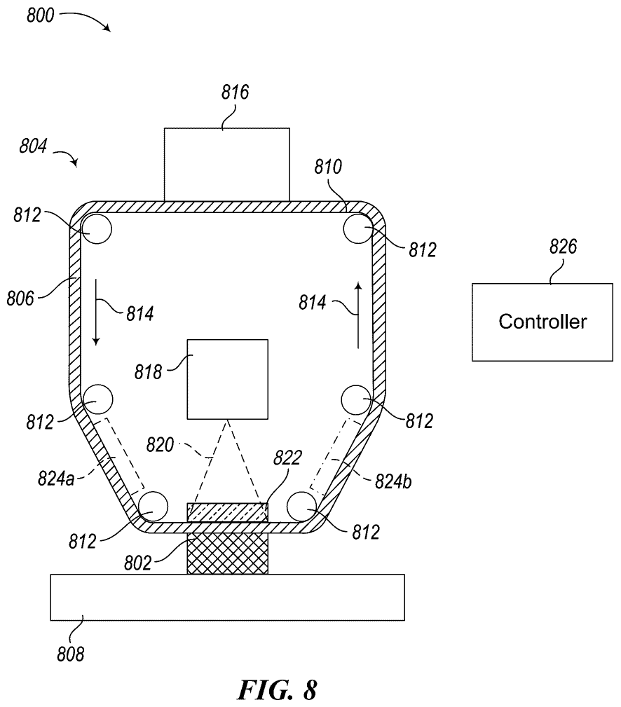

FIG. 8 illustrates a representative example of an additive manufacturing device 800 (“device 800”) configured in accordance with embodiments of the present disclosure. The device 800 can be used to fabricate any embodiment of the additively manufactured objects described herein. For example, the device 800 can be used to produce an additively manufactured object in accordance with operation 702 of the method 700 of FIG. 7.

As shown in FIG. 8, the device 800 is used to fabricate an additively manufactured object 802 (“object 802”). The device 800 includes a printer assembly 804 configured to deposit resin 806 on a build platform 808 (e.g., a tray, plate, film, sheet, or other planar substrate) to form the object 802. The printer assembly 804 includes a carrier film 810 configured to deliver the resin 806 to the build platform 808. The carrier film 810 can be a flexible loop of material having an outer surface and an inner surface. The outer surface of the carrier film 810 can adhere to and carry a thin layer of the resin 806. The inner surface of the carrier film 810 can contact one or more rollers 812 that rotate to move the carrier film 810 in a continuous loop trajectory, e.g., as indicated by arrows 814.

The printer assembly 804 can also include a resin source 816 (shown schematically) configured to apply the resin 806 to the carrier film 810. In the illustrated embodiment, the resin source 816 is located at the upper portion of the printer assembly 804 near an upper horizontal segment of the carrier film 810. In other embodiments, however, the resin 806 can be positioned at a different location in the printer assembly 804. The resin source 816 can include nozzles, ports, reservoirs, etc., that deposit the resin 806 onto the outer surface of the carrier film 810. The resin source 816 can also include one or more blades (e.g., doctor blades, recoater blades) that smooth the deposited resin 806 into a relatively thin, uniform layer. In some embodiments, the resin 806 is formed into a layer having a thickness within a range from 200 microns to 300 microns.

The resin 806 can be carried by the carrier film 810 toward the build platform 808. In the illustrated embodiment, the build platform 808 is located below the printer assembly 804 near a lower horizontal segment of the carrier film 810. In other embodiments, however, the build platform 808 can be positioned at a different location relative to the printer assembly 804. The printer assembly 804 includes a light source 518 (e.g., a projector or light engine) that outputs light 820 (e.g., UV light) having a wavelength configured to cure the resin 806 partially or fully. The carrier film 810 can be optically transparent so that the light 820 from the light source 818 passes through the carrier film 810 and onto the portion of the resin 806 above the build platform 808, thus forming a layer of cured resin 806 onto the build platform 808 and/or a previously formed portion of the object 802. The light 820 can be patterned or scanned in a suitable pattern corresponding to the desired cross-section geometry for the object 802. Optionally, a transparent plate 822 can be disposed between the light source 818 and the carrier film 810 to guide the carrier film 810 into a specific position (e.g., height) relative to the build platform 808.

Once the object cross-section has been formed, the build platform 808 can be lowered by a predetermined amount to separate the cured resin from the carrier film 810. The remaining, uncured resin 806 can be carried by the carrier film 810 away from the build platform 808 and back toward the resin source 816. The resin source 816 can deposit additional resin 806 onto the carrier film 810 and/or smooth the resin 806 to re-form a uniform layer of resin 806 on the carrier film 810. The resin 806 can then be recirculated back to the build platform 808 to fabricate an additional layer of the object 802. This process can be repeated to iteratively build up individual object layers on the build platform 808 until the object 802 is complete. The object 802 and build platform 808 can then be removed from the device 800 for post-processing.

In some embodiments, the device 800 is used in a high temperature lithography process utilizing a highly viscous resin. Accordingly, the printer assembly 804 can include one or more heat sources (heating plates, infrared lamps, etc.) for heating the resin 806 to lower the viscosity to a range suitable for additive manufacturing. For example, the printer assembly 804 can include a first heat source 824a positioned against the segment of the carrier film 810 before the build platform 808, and a second heat source 824b positioned against the segment of the carrier film 810 after the build platform 808. Alternatively, or in combination, the printer assembly 804 can include heat sources at other locations.

The device 800 also includes a controller 826 (shown schematically) that is operably coupled to the printer assembly 804 and build platform 808 to control the operation thereof. The controller 826 can be or include a computing device including one or more processors and memory storing instructions for performing the additive manufacturing operations described herein. For example, the controller 826 can receive a digital 3D model of the object 802 to be fabricated, determine a plurality of object cross-sections to build up the object 802 from the resin 806, and can transmit instructions to the light source 818 to output light 820 to form the object cross-sections. As another example, the controller 826 can also determine and control other operational parameters, such as the positioning of the build platform 808 (e.g., height) relative to the carrier film 810, the movement speed and direction of the carrier film 810, the amount of resin 806 deposited by the resin 806, the thickness of the resin layer on the carrier film 810, and/or the amount of heating applied to the resin 806.

Although FIG. 8 illustrates a representative example of an additive manufacturing device, this is not intended to be limiting, and the embodiments described herein can be used in combination with other types of additive manufacturing devices (e.g., vat-based systems) and/or other types of additive manufacturing processes (e.g., material jetting, binder jetting, FDM, powder bed fusion, sheet lamination, directed energy deposition).

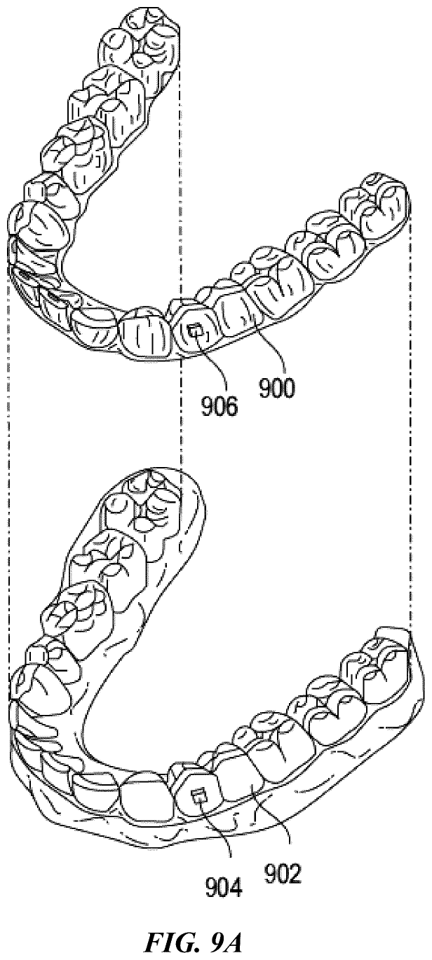

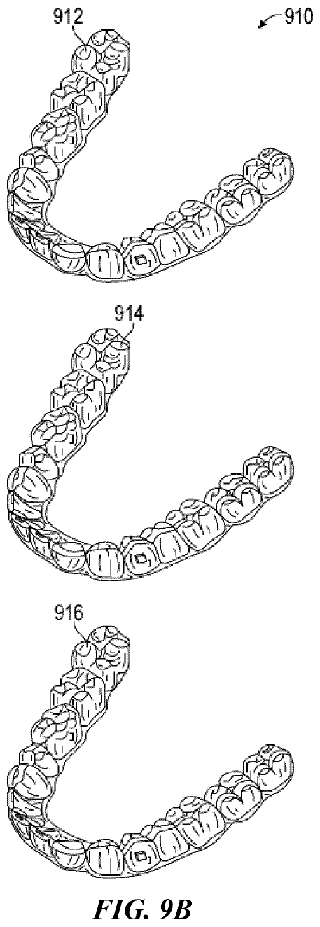

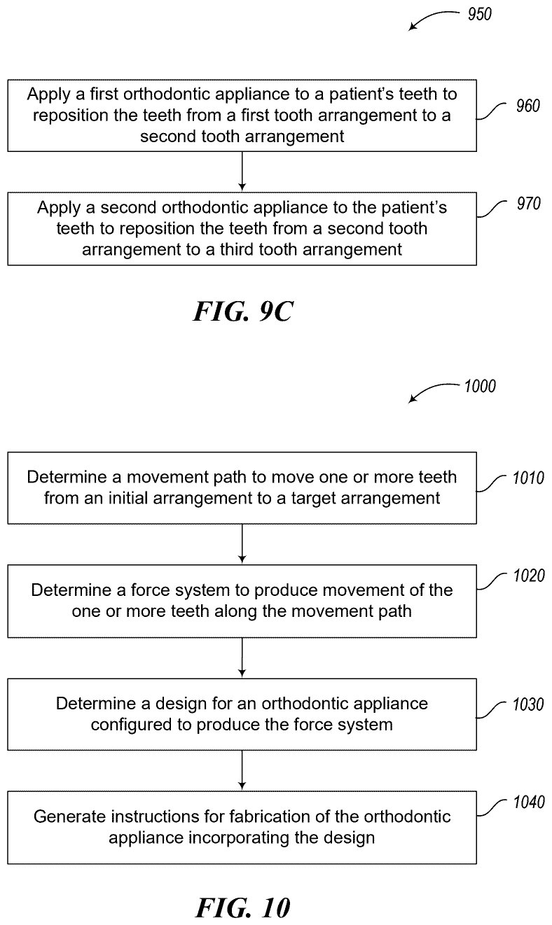

FIG. 9A illustrates a representative example of a tooth repositioning appliance 900 configured in accordance with embodiments of the present disclosure. The appliance 900 can be manufactured and post-processed using any of the systems, methods, and devices described herein. The appliance 900 (also referred to herein as an “aligner”) can be worn by a patient to achieve an incremental repositioning of individual teeth 902 in the jaw. The appliance 900 can include a shell (e.g., a continuous polymeric shell or a segmented shell) having teeth-receiving cavities that receive and resiliently reposition the teeth. The appliance 900 or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using rapid prototyping fabrication techniques, from a digital model of an appliance. In some embodiments, direct fabrication involves forming an object (e.g., an appliance or a portion thereof) without using a physical template (e.g., mold, mask etc.) to define the object geometry. Though the discussion herein refers to “aligners,” any or all the discussion herein can refer to clear retainers used to retain a person's dentition at a target, final, intermediate, etc. stage of a treatment plan.

The appliance 900 can fit over all teeth present in an upper or lower jaw, or less than all the teeth. The appliance 900 can be designed specifically to accommodate the teeth of the patient (e.g., the topography of the tooth-receiving cavities matches the topography of the patient's teeth) and may be fabricated based on positive or negative models of the patient's teeth generated by impression, scanning, and the like. Alternatively, the appliance 900 can be a generic appliance configured to receive the teeth, but not necessarily shaped to match the topography of the patient's teeth. In some cases, only certain teeth received by the appliance 900 are repositioned by the appliance 900 while other teeth can provide a base or anchor region for holding the appliance 900 in place as it applies force against the tooth or teeth targeted for repositioning. In some cases, some, most, or even all the teeth can be repositioned at some point during treatment. Teeth that are moved can also serve as a base or anchor for holding the appliance as it is worn by the patient. In preferred embodiments, no wires or other means are provided for holding the appliance 900 in place over the teeth. In some cases, however, it may be desirable or necessary to provide individual attachments 904 or other anchoring elements on teeth 902 with corresponding receptacles 906 or apertures in the appliance 900 so that the appliance 900 can apply a selected force on the tooth. Representative examples of appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 9B illustrates a tooth repositioning system 910 including a plurality of appliances 912, 914, 916, in accordance with embodiments of the present disclosure. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 910 can include a first appliance 912 corresponding to an initial tooth arrangement, one or more intermediate appliances 914 corresponding to one or more intermediate arrangements, and a final appliance 916 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 9C illustrates a method 950 of orthodontic treatment using a plurality of appliances, in accordance with embodiments of the present disclosure. The method 950 can be practiced using any of the appliances or appliance sets described herein. At operation 960, a first orthodontic appliance is applied to a patient's teeth to reposition the teeth from a first tooth arrangement to a second tooth arrangement. At operation 970, a second orthodontic appliance is applied to the patient's teeth to reposition the teeth from the second tooth arrangement to a third tooth arrangement. The method 950 can be repeated as necessary using any suitable number and combination of sequential appliances to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied to compensate for any inaccuracies or limitations of the appliance.

FIG. 10 illustrates a method 1000 for designing an orthodontic appliance, in accordance with embodiments of the present disclosure. The method 1000 can be applied to any embodiment of the orthodontic appliances described herein. Some or all the steps of the method 1000 can be performed by any suitable data processing system or device, e.g., one or more processors configured with suitable instructions.

At operation 1010, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

At operation 1020, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully formed suture. Thus, in juvenile patients and others without fully closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients can require lower forces to expand the suture than older patients, as the suture has not yet fully formed.

At operation 1030, a design for an orthodontic appliance configured to produce the force system is determined. The design can include the appliance geometry, material composition, and/or material properties, and can be determined in various ways, such as using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, Calif. For creating finite element models and analyzing them, program products from several vendors can be used, including finite element analysis packages from ANSYS, Inc., of Canonsburg, Pa., and SIMULIA (Abaqus) software products from Dassault Systèmes of Waltham, Mass.

Optionally, one or more designs can be selected for testing or force modeling. As noted above, a desired tooth movement, as well as a force system required or desired for eliciting the desired tooth movement, can be identified. Using the simulation environment, a candidate design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

At operation 1040, instructions for fabrication of the orthodontic appliance incorporating the design are generated. The instructions can be configured to control a fabrication system or device to produce the orthodontic appliance with the specified design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Although the above steps show a method 1000 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 1000 may be performed with any suitable fabrication system or device, such as the embodiments described herein. Some of the steps may be optional, and the order of the steps can be varied as desired.

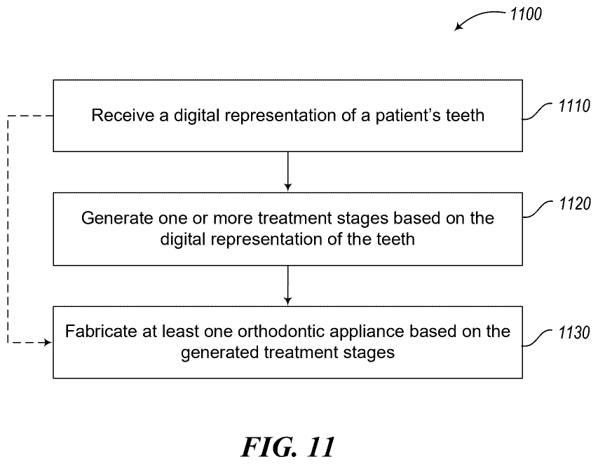

FIG. 11 illustrates a method 1100 for digitally planning an orthodontic treatment and/or design or fabrication of an appliance, in accordance with embodiments. The method 1100 can be applied to any of the treatment procedures described herein and can be performed by any suitable data processing system.

At operation 1110, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

At operation 1120, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

At operation 1130, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 11, design and/or fabrication of an orthodontic appliance, and perhaps a particular orthodontic treatment, may include use of a representation of the patient's teeth (e.g., including receiving a digital representation of the patient's teeth (operation 1110)), followed by design and/or fabrication of an orthodontic appliance based on a representation of the patient's teeth in the arrangement represented by the received representation.

As noted herein, the techniques described herein can be used for the direct fabrication of dental appliances, such as aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

The techniques used herein can also be used to manufacture attachment fabrication templates, e.g., appliances used to position prefabricated attachments on a person's teeth in accordance with one or more aspects of a treatment plan. Examples of attachment fabrication templates can be found at least in: U.S. application Ser. No. 17/249,218, entitled, “Flexible 3D Printed Orthodontic Device,” filed Feb. 24, 2021; U.S. application Ser. No. 16/366,686, entitled, “Dental Attachment Placement Structure,” filed Mar. 27, 2019; U.S. application Ser. No. 15/674,662, entitled, “Devices and Systems for Creation of Attachments,” filed Aug. 11, 2017; U.S. Pat. No. 11,103,330, entitled, “Dental Attachment Placement Structure,” filed Jun. 14, 2017; U.S. application Ser. No. 14/963,527, entitled, “Dental Attachment Placement Structure,” filed Dec. 9, 2015; U.S. application Ser. No. 14/939,246, entitled, “Dental Attachment Placement Structure,” filed Nov. 12, 2015; U.S. application Ser. No. 14/939,252, entitled, “Dental Attachment Formation Structures,” filed Nov. 12, 2015; and U.S. Pat. No. 9,700,385, entitled, “Attachment Structure,” filed Aug. 22, 2014; all of which are incorporated by reference herein in their entirety.

The techniques described herein can be used to make incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled, “Releasable Palatal Expanders,” filed Apr. 10, 2019; U.S. application Ser. No. 16/022,552, entitled, “Devices, Systems, and Methods for Dental Arch Expansion,” filed Jun. 28, 2018; U.S. Pat. No. 11,045,283, entitled, “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled, “Methods and Apparatuses for Customizing a Rapid Palatal Expander,” filed Dec. 4, 2017; and U.S. Pat. No. 7,192,273, entitled, “System and Method for Palatal Expansion,” filed Aug. 7, 2003; all of which are incorporated by reference herein in their entirety.

### EXAMPLES

The following examples are given for the purpose of illustrating various embodiments of the disclosure and are not meant to limit the present disclosure in any fashion. The present examples, along with the methods described herein are presently representative of some embodiments, are exemplary, and are not intended as limitations on the scope of the disclosure. Changes therein and other uses which are encompassed within the spirit of the disclosure as defined by the scope of the claims will occur to those skilled in the art.

### Example 1

### Catalytic Depolymerization of Walnut Shell

5 g granular or powdered walnut shell, 1 g powdered Co₃O₄ catalyst, and 200 ml acetone were placed in a Parr reactor. The mixture was stirred at 300-700 rpm. The depolymerization reaction was carried out at 500 psi and 190° C., with 6% O₂ in N₂ for 6 hours. Crude oil product and residual solids were collected. Residual solids containing walnut shell and catalyst residues were separated from the crude oil by sieves. 1.8 g crude oil comprising syringic acid and syringaldehyde was obtained (2.7% yield).

### Example 2

### Catalytic Depolymerization of Walnut Shell

5 g granular or powdered walnut shell, 1 g powdered Co/silica catalyst, and 200 ml acetone were placed in a Parr reactor. The mixture was stirred at 300-700 rpm. The depolymerization reaction was carried out at 500 psi and 190° C., with 6% O₂ in N₂ for 1-2 hours. Crude oil product and residual solids were collected. Residual solids containing walnut shell and catalyst residues were separated from the crude oil by sieves. 1.8 g crude oil comprising syringic acid and syringaldehyde was obtained (2.7% yield).

### Example 3

### Catalytic Depolymerization of Walnut Shell

5 g granular or powdered walnut shell, 2 g powdered Co/silica catalyst, and 200 ml acetone were placed in a Parr reactor. The mixture was stirred at 300-700 rpm. The depolymerization reaction was carried out at 500 psi and 190° C., with 6% O₂ in N₂ for 6 hours. Crude oil product and residual solids were collected. Residual solids containing walnut shell and catalyst residues were separated from the crude oil by sieves. 1.8 g crude oil comprising syringic acid and syringaldehyde was obtained (2.1% yield).

### Example 4

### Catalytic Depolymerization of Walnut Shell

1 kg granular or powdered walnut shell, 1 g powdered Co/silica catalyst, and 9 L acetonitrile were placed in a Parr reactor. The mixture was stirred at 300-700 rpm. The depolymerization reaction was carried out at 500 psi and 190° C., with 6% O₂ in N₂ for 1 h. Crude oil product comprising a mixture of syringic acid, syringaldehyde, vanillin, and vanillic acid and residual solids were collected. Residual solids containing walnut shell and catalyst residues were separated from the crude oil by sieves. 207 g crude oil comprising syringic acid (1.5% yield) and syringaldehyde was obtained (1.5% yield).

### Example 5

Non-catalytic Depolymerization of Walnut Shell with Batch-Wise Oxygen Feed

Powdered walnut shell and acetonitrile were placed in a Parr reactor and stirred at 1200 rpm. After introducing an oxygen source containing 8% oxygen gas balanced with N₂ into the reactor to fill the headspace, the depolymerization reaction was carried out at 500 psi and 190° C. Once the oxygen inside the reactor was depleted, the reactor was opened, and the assay was analyzed to identify components in the crude oil.

To further increase the yield, additional oxygen source was introduced into the headspace to initiate another reaction cycle. The depolymerization reaction was repeated four times. The results of conversion yield after each cycle are summarized in Table 1.

TABLE 1
Aromatic Compounds Identified in Crude Oil Obtained
from Depolymerization of Walnut Shell with 8% Oxygen

| Aromatic | Assay yields after each cycle (wt %) | | | |
|---|---|---|---|---|
| Compounds | 1st Cycle | 2nd Cycle | 3rd Cycle | 4th Cycle |
| Syringic acid | 0.3 | 0.7 | 1.1 | 1.3 |
| Syringaldehyde | 0.3 | 0.7 | 1.0 | 0.9 |
| Vanillic acid | 0.1 | 0.3 | 0.5 | 0.6 |
| Vanillin | 0.2 | 0.3 | 0.4 | 0.6 |

### Example 6

Non-catalytic Depolymerization of Walnut Shell with Batch-Wise Air Feed

Powdered walnut shell and acetonitrile were placed in a Parr reactor and stirred at 1200 rpm. After introducing air into the reactor to fill the headspace, the depolymerization reaction was carried out at 500 psi and 190° C. Once the oxygen inside the reactor was depleted, the reactor was opened, and the assay was analyzed to identify components in the crude oil.

To further increase the yield, additional air was introduced into the headspace to initiate another reaction cycle. The depolymerization reaction was repeated four times. The results of conversion yield after each cycle are summarized in Table 2.

TABLE 2
Aromatic Compounds Identified in Crude Oil Obtained
from Depolymerization of Walnut Shell with Air

| Aromatic | Assay yields after each cycle (wt %) | | | |
|---|---|---|---|---|
| Compounds | 1st Cycle | 2nd Cycle | 3rd Cycle | 4th Cycle |
| Syringic acid | 0.9 | 1.8 | 2.1 | 2.3 |
| Syringaldehyde | 0.9 | 1.0 | 0.9 | 1.0 |
| Vanillic acid | 0.5 | 1.0 | 1.3 | 1.5 |
| Vanillin | 0.4 | 0.6 | 0.7 | 0.75 |

### Example 7

Non-catalytic Depolymerization of Walnut Shell with Continuous Oxygen Feed

400 g granular or powdered walnut shell and 2 L acetonitrile were placed in a 4 L reactor. The mixture was stirred at 1200 rpm. An oxygen source containing 8% oxygen gas balanced with N₂ gas was continuously flowed into the reactor, while the depolymerization reaction proceeded at 500 psi and 190° C. for 16 h.

The crude oil product comprising syringol along with syringic acid, syringaldehyde, vanillin, and vanillic acid and residual solids was collected. The residual solids containing walnut shell were separated from the crude oil by sieves. The crude oil was then purified to afford 2.3 g of syringol.

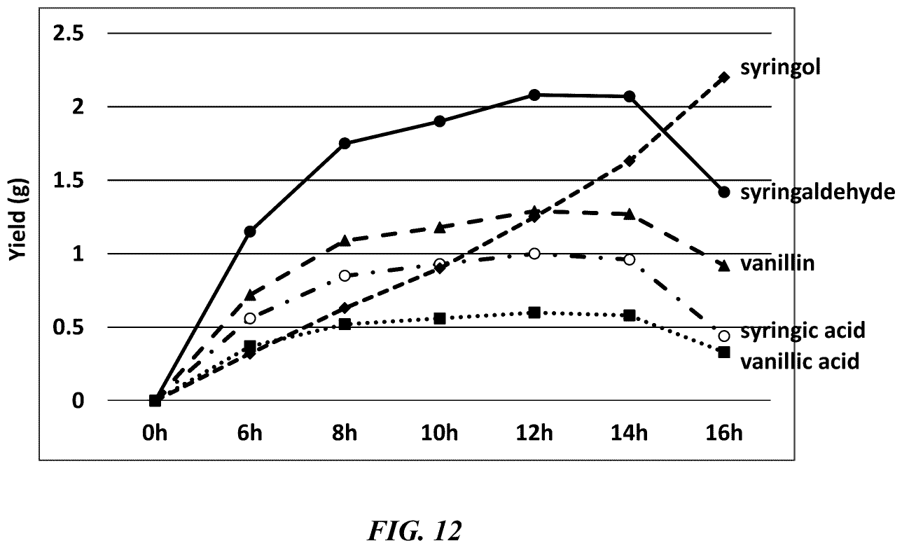

FIG. 12 illustrates the effect of continuous oxygen feed on the yield of aromatic compounds during the depolymerization of lignin in walnut shell. The continuous oxygen feed has a significant impact on syringol formation, with its yield steadily increasing over time. In contrast, the amounts of syringaldehyde, syringic acid, vanillin, and vanillic acid initially increased as lignin depolymerization progressed, peaked at 14 hours, and then declined with further reaction time.

### Example 8

### Chemical Upgrading of Syringaldehyde to Syringol

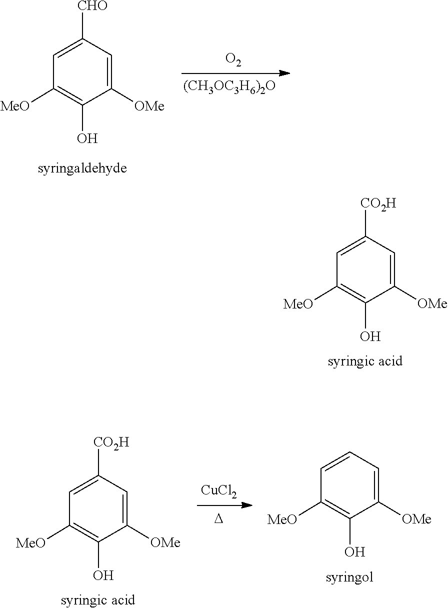

Syringaldehyde (2 g) was treated with bis(methoxypropyl) ether at 200 psi with 8% O₂ in N₂ for 5 days, affording syringic acid with 50% yield.

Syringic acid (1 g) in 600 ml water was decarboxylated using a copper-based catalyst at 170° C.-200° C. for 6-24 hours to convert syringic acid into syringol. Table 3 summarizes the conversion yields using different copper-based catalysts.

TABLE 3
Decarboxylation of Syringic Acid Using Copper-Based Catalysts

| Syringic | | Catalyst | Reaction | Syringol |
|---|---|---|---|---|
| Acid | Catalyst | Amount | Conditions | Yield |
| 1 g | CuCl₂ | 2.5 eq | 200° C., 6 hours | 33% |
| 1 g | CuSO₄ | 2.5 eq | 190° C., 6 hours | 84% |
| 1 g | Cu(OAc)₂ | 2.5 eq | 190° C., 6 hours | 60% |
| 10 | CuSO₄ | 0.1 eq | 170° C., 24 hours | 85% |

### Example 9

### Chemical Upgrading of Syringaldehyde to Syringol

To a solution of syringaldehyde (1 g, 5.5 mmol, 1 equiv) in dimethyl sulfoxide (7 mL) was added a solution of sodium dihydrogen phosphate (0.2 g, 1.6 mmol, 0.3 equiv) in water (2 mL). Then a solution of sodium chlorite (1.1 g, 2.2 equiv) in water (3 mL) was added slowly, keeping the temperature below 60° C. After the addition was completed, the reaction mixture was stirred at 60° C. for 12 h. Additional sodium chlorite (0.5 g, 1.0 equiv) in water (1 mL) was then added slowly, and the mixture was stirred at 60° C. for 4 more hours. LC-MS analysis indicated 90%-99% conversion. The mixture was cooled to room temperature and diluted with 2 N hydrochloric acid (1 mL) and ethyl acetate (20 mL). The organic layer was separated and the aqueous layer was extracted with ethyl acetate (2×20 mL). The organic layers were combined, washed with brine (10 mL), dried over sodium sulfate, and concentrated under reduced pressure. The crude syringic acid was purified over silica gel (30 g) using 10% methanol in ethyl acetate to give syringic acid as an off-white solid (0.7 g, 64% yield). 1H NMR (400 MHZ, DMSO-d6) δ=12.6 (br, 1H), 9.18 (s, 1H), 7.21 (s, 2H), 3.80 (s, 6H).

Syringic acid was then decarboxylated using a copper-based catalyst at 190° C.-200° C. for 6 hours to convert syringic acid into syringol.

### Example 10

### Chemical Upgrading of Syringaldehyde to Syringol

Syringaldehyde (1.8 g) was treated with silver nitrate (2 equiv) and potassium hydroxide (8 equiv) in water at 60° C. for 1 hour, resulting in a 14% conversion to syringic acid. Syringic acid was then decarboxylated using a copper-based catalyst at 190° C.-200° C. for 6 hours to convert syringic acid into syringol.

### Example 11

### Chemical Upgrading of Syringaldehyde to Syringol

Syringaldehyde (1 g) was treated with sodium chlorite (1.2 equiv) and sodium dihydrogen phosphate (0.3 equiv) in a mixture of dimethyl sulfoxide and water at 40° C. for 18 hours, resulting in 17% conversion to syringic acid. Syringic acid was then decarboxylated using a copper-based catalyst at 190° C.-200° C. for 6 hours to convert syringic acid into syringol.

### Example 12

### Purification Crude Syringol Obtained From Chemical Upgrading

A 5 g crude syringol was suspended in 20 mL of water and 10 mL of ethyl acetate. The pH of the mixture was determined to be 3.02. The pH was adjusted to 10.0 using NaOH (1.27 g). The mixture was concentrated under vacuum to remove the ethyl acetate. The remaining aqueous phase was extracted with 10 mL of MTBE. The phases were separated and the aqueous phase was extracted with 10 mL of MTBE. The organic phase contained 60-70 wt % syringol balanced with 30-40 wt % guaiacol, while vanillin and syringaldehyde were in the aqueous phase. Syringol (60-70 wt %) was separated from guaiacol with the distillation column. After distillation, the concentration of syringol reached up to 99 wt %.

### Example 13

### Purification Crude Syringol Obtained From Lignin Direct Pyrolysis Depolymerization (A) Selective Solvent Extraction

200 mL of water was added to 20 g of crude syringol. The pH of the resulting solution was adjusted to 13.0 using KOH pellets. The solution was first extracted with 100 mL of MTBE, and both phases were sampled and analyzed using GCMS. The MTBE volume was increased in 100 mL increments, ending at 600 mL. Both phases were sampled and analyzed after each MTBE volume increase. The MTBE phase was discarded and replaced with 100 mL of fresh MTBE and the pH was adjusted to 9.5 using HCl (conc.). The extraction process was performed as above, proceeding from 100 mL to 600 mL MTBE in 100 mL increments. An emulsion formed during the first four extractions at pH 9.5 and phase separation was slow. The emulsion dissipated after the fifth portion of MTBE was added. Both phases were sampled and analyzed after each extraction. The final organic phase was dried over sodium sulfate and concentrated under vacuum to obtain 2.9 g of syringol enriched bio-oil in dark color (15% yield).

### (B) Distillation

Oldershaw distillation column with 10 plates was used to separate syringol from the enriched syringol bio-oil was used. The vacuum pressure was varied between 2 mbar and 6 mbar. The syringol enriched bio-oil was placed in the pot at the bottom of the distillation column, and then heated to 115-140° C. Vapor-liquid equilibrium was reached at each plate and the distillate temperature at the top was recorded as well. The distillate was condensed into 5-10 fractions and each fraction was analyzed.

### Example 14

Producing Syringol from Crude Oil Obtained by Depolymerization of Walnut Shell

Crude oil containing syringic acid (6 g, 4.9 wt % concentration) was treated with copper (II) sulfate (0.05 equiv. relative to syringic acid) in water at 170° C. for 24 hours, achieving 99% conversion of syringic acid to syringol with 17 wt % dimer formation. The reaction mixture was then extracted using MTBE to yield 2 g of crude syringol (14 wt % concentration).

### Example 15

Producing Syringol from Crude Oil Obtained by Depolymerization of Walnut Shell

Crude oil containing syringic acid (6 g, 5.0 wt % concentration) was treated with copper (II) sulfate (0.1 equiv. relative to syringic acid) in water at 170° C. for 24 hours, achieving 99% conversion of syringic acid to syringol with 3 wt % dimer formation. The reaction mixture was then extracted using MTBE to yield 3.2 g crude syringol (15 wt % concentration).

### Example 16

### Methylation of Pyrogallol

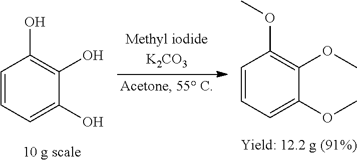

A slurry of 10 g of pyrogallol (79.2 mmol, 1.0 eq), 29.6 mL of iodomethane (475.7 mmol, 6.0 eq), and 65.7 g of potassium carbonate (475.7 mmol, 6.0 eq) in 100 mL of acetone was heated to 50° C. under nitrogen. The slurry was collected the next morning; it was dissolved in 300 mL of water and extracted with 2×100 mL of MTBE. The aqueous phase was discarded, and the combined organic phases were dried over sodium sulfate and concentrated under vacuum to obtain 12.87 g of dark oil. The oil crystallized soon after standing at room temperature

### Example 17

### Demethylation of 1,2,3-Trimethoxybenzene

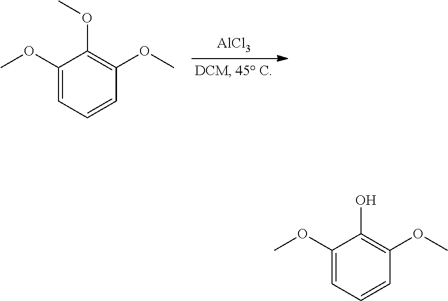

To a dry and clean 3-neck 3 L round-bottomed flask was added 1,2,3 trimethoxybenzene (100 g, 594.5 mmol) under argon atmosphere and the anhydrous DCM (1.2 L) was added to the flask. Then the mixture was stirred at room temperature until all the starting material was dissolved. Under the flow of argon, AlCl₃ (118.90 g, 891.7 mmol, 1.5 eq) was added in a portion wise (in three portions) over a period of 15 min. After the addition, the reaction mixture was placed on a heating mantle and a condenser was attached with water circulation and controlled the temperature to 45° C. After 16 hours, there was no sign of starting material. Then the reaction mixture was cooled down to room temperature and the flask was placed in an ice bath to cool down to 0° C. Then water (1 L) was added slowly dropwise to quench the reaction (observed severe exothermic nature while quenching). The reaction mixture was transferred into a separatory funnel and layers were separated. The organic layer was discarded after making sure that there was no product. The aqueous layer was acidified to pH=1 and extracted with MTBE (3×500 mL) and the combined organic layers were dried over Na₂SO₄ (100 g). The organic layer was filtered and concentrated on a rotavapor to get crude syringol (104.21 g) which was taken to the crystallization step. Various reaction conditions were screened and Table 4 provides a summary of the screening results.

TABLE 4
Reaction Condition Screening Results

| | | | | | Concentration of | |
|---|---|---|---|---|---|---|
| | | | AlCl₃ | | 1,2,3- | |
| Entry | Scale | | (equiv) | Temperature | trimethoxylbenzene | Result |
| 1 | 50 | mg | 0.75 | Room temp | 1M | Traces of desired product |
| 2 | 50 | mg | 0.75 | 50° C. | 1M | Desired product observed |
| | | | | | | with undesired isomer |
| 3 | 50 | mg | 0.75 | 40° C. | 1M | After 24 h, no progress |
| 4 | 50 | mg | 0.75 | 45° C. | 3M | After 24 h, no progress |
| 5 | 50 | mg | 1.5 (0.75 eq | 45° C. | 3M | Reaction completed to 100% |
| | | | added twice) | | | conversion and only traces |
| | | | | | | of impurities |
| 6 | 50 | mg | 1.05 | 45° C. | 3M | After 36 h, no progress. |
| 7 | 50 | mg | 1.5 | 45° C. | 3M | After 36 h, no progress |
| 8 | 100 | mg | 1.5 (added | 45° C. | 3M | Reaction completed to 100% |
| | | | over 15 min) | | | conversion with the major |
| | | | | | | product |
| 9 | 10 | g | 1.5 (added | 45° C. | 3M | Reaction completed to 100% |
| | | | over 15 min) | | | conversion, observed major |
| | | | | | | product, purified by column |
| | | | | | | chromatograph, 73% yield |
| 10 | 100 | g | 1.5 (added | 45° C. | 3M | Crud yield: 104.21 g |
| | | | over 15 min) | | | |
| 11 | 3 | g | 1.1 | 90° C. | 3M | 88% yield of syringol |

### Example 18

Demethylation of 1,2,3-Trimethoxybenzene Using BF₃·OEt₂

To a dry and clean 3-neck 3 L round-bottomed flask was added 1,2,3 trimethoxybenzene (0.168 g, 1 mmol) under argon atmosphere and the anhydrous 1,4-dioxane was added to the flask. Then the mixture was stirred at room temperature until all the starting material was dissolved. Under the flow of argon, BF₃·OEt₂ (0.142 g, 1 mmol, 1 eq) and KI (0.166 g, 1 eq) were added in a portion wise (in three portions) over a period of 15 min. After the addition, the reaction mixture was placed on a heating mantle, and a condenser was attached with water circulation and controlled the temperature to 40° C. After 48 hours, there was no sign of starting material. Then the reaction mixture was cooled down to room temperature and then flask was placed in an ice bath to cool down to 0° C. Then water was added slowly dropwise to quench the reaction (observed severe exothermic nature while quenching). The reaction mixture was transferred into a separatory funnel and layers were separated. The organic layer was discarded after making sure that there was no product. The aqueous layer was acidified to pH=1 and extracted with MTBE (3×500 mL) and combined organic layers were dried over Na₂SO₄ (100 g). The organic layer was filtered and concentrated on a rotavapor to get crude syringol which was taken to the crystallization step.

### Example 19

### Demethylation of 1,2,3-Trimethoxybenzene Using Me₃Sil

To a dry and clean 3-neck 3 L round-bottomed flask was added 1,2,3 trimethoxybenzene (0.168 g, 1 mmol) under argon atmosphere and the anhydrous DCM (0.32 mL) was added to the flask. Then the mixture was stirred at room temperature until all the starting material was dissolved. Under the flow of argon, MesSil (0.2 g, 1 mmol, 1 eq) was added in a portion wise (in three portions) over a period of 15 min. After the addition, the reaction mixture was placed on a heating mantle and a condenser was attached with water circulation and controlled the temperature to 40° C. After 48 hours, there was no sign of starting material. Then the reaction mixture was cooled down to room temperature and the flask was placed in an ice bath to cool down to 0° C. Then water (1 L) was added slowly dropwise to quench the reaction (observed severe exothermic nature while quenching). The reaction mixture was transferred into a separatory funnel and layers were separated. The organic layer was discarded after making sure that there was no product. The aqueous layer was acidified to pH=1 and extracted with MTBE (3×500 mL) and the combined organic layers were dried over Na₂SO₄ (100 g). The organic layer was filtered and concentrated on a rotavapor to get crude syringol (0.14 g) which was taken to the crystallization step.

### Example 20

### Crystallization of Syringol

100 mg crude was dissolved in a minimal amount of DCM (0.2 mL) and heptane (2 mL) was added and agitated at 0° C. and kept at the same temperature for 1 h (only observed waxy residue). Then cooled down to −10° C. and kept for 2 hours. Crystallization was observed. 42 mg crystals were collected.

The same crystallization procedure was also used with Me-THF as the solvent instead of DCM. Again, pure syringol was obtained. The mass of the crystals was 59 mg. There was still some syringol yet to crystallize from the solution.

Scaled-up crystallization: 10 g crude was dissolved in a minimal amount of Me-THF (9.5 mL) and heptane (30 mL) was added and then cooled down to −10° C. and kept for 2 hours. 7.2 g pure syringol was collected. 94 g crude was dissolved in minimal amount of Me-THF (89 mL) and heptane (282 mL) was added and then cooled down to −10° C. and kept for 2 hours. 58.9 g pure syringol was collected. Solvent system used and crystallization yield is summarized in Table 5 below:

TABLE 5
A summary of solvent system used and crystallization yield

| Solvent | Crude Scale | Crystallization Yield |
|---|---|---|
| Me-THF (1 V) + heptane (3 V) | 10 g | 7.2 g (72%) |
| Me-THF (1 V) + heptane (3 V) | 94 g | 58.9 g (63%) |

### Example 21

### Purification of Syringol

1,2,3-Trimethoxybenzene (150 g) was treated with 1.1 eq AlCl₃ at 90° C. for 2 h as described above to give a mixture of syringol, diol, and 2,3-dimethyl isomer. Aqueous work-up gave crude syringol (130.5 g, 95% yield). Crude syringol was purified via recrystallization under different conditions. The results are summarized in Table 6.

TABLE 6
Purification of Syringol

| | Syringol | MTBE | Heptane | Yield | Purity | Temp |
|---|---|---|---|---|---|---|
| | (g) | (vol) | (vol) | (%) | (%) | (° C.) |
| 1 | 32 | 2 | 4 | 52 | 98 | 18-20 |
| 2 | 1 | 2 | 8 | 55 | 98 | 18-20 |
| 3 | 2 | 1 | 4 | 65 | 98 | 18-20 |
| 4 | 4 | 0.5 | 2 | 83 | 88 | 18-20 |
| 5 | 2 | 1 | 6 | 64 | 98 | 18-20 |
| 6 | 2 | 1 | 8 | 63 | 98 | 18-20 |
| 7 | 10 | 1 | 4 | 80 | 92 | 5 |
| 8 | 10 | 1 | 2 | 72 | 93 | 5 |
| 9 | 10 | 0.8 | 3.2 | 73 | 92 | 20 |
| 10 | 10 | 0.9 | 3.6 | 71 | 93 | 20 |
| 11 | 10 | 1 | 3 | 67 | 96 | 20 |
| 12 | 10 | 1.5 | 3 | 66 | 98 | 5 |

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for producing syringol from a biomass, comprising:
introducing a composition comprising the biomass and a solvent into a reactor;
feeding an oxygen source into the reactor;
heating the composition at a temperature below 200° C. while continuously feeding the oxygen source into the reactor to depolymerize lignin in the biomass, thereby yielding a depolymerized lignin composition comprising syringol; and
obtaining syringol from the depolymerized lignin composition.

2. The method of claim 1, wherein the oxygen source is fed into the reactor at a rate of 0.2 L/min to 5 L/min.

3. The method of claim 1, wherein the depolymerized lignin composition further comprises syringaldehyde, syringic acid, vanillin and vanillic acid.

4. A method for producing syringol from a biomass, comprising:
introducing a composition comprising the biomass and a solvent into a reactor;
feeding a first amount of an oxygen source into the reactor;
heating the composition at a temperature below 200° C. until oxygen in the first amount of oxygen source is depleted to depolymerize lignin in the biomass, thereby yielding a depolymerized lignin composition comprising syringaldehyde and syringic acid; and
converting syringaldehyde and syringic acid into syringol.

5. The method of claim 4, further comprising steps of:
(a) feeding a second amount of the oxygen source into the reactor;
(b) heating the composition at a temperature blow 200° C. until oxygen in the second amount of the oxygen source is depleted; and
(c) optionally repeating steps (a) and (b) prior to converting syringaldehyde and syringic acid into syringol.

6. The method of claim 5, wherein steps (a) and (b) are repeated one or more times.

7. (canceled)

8. The method of claim 1, wherein;
(A) the solvent comprises water, anisole, methanol, ethanol, acetone, acetonitrile, methylene chloride (MEC), perchloroethylene (PCE), trichloroethylene (TCE), polyethylene glycol, sulfolane, γ-valerolactone (GVL), N-methylpyrrolidone (NMP) or a mixture thereof; or
(B) the solvent comprises supercritical water, supercritical methanol or supercritical ethanol.

9. (canceled)

10. The method of claim 1, wherein the oxygen source comprises an oxygen gas or air.

11. The method of claim 10, wherein the oxygen gas is balanced with a nitrogen gas.

12. The method of claim 1, wherein the oxygen source comprises at least 2 vol % of oxygen.

13. The method of claim 1, wherein the depolymerization of lignin is carried out at a pressure below 600 psi.

14. The method of claim 1, wherein the depolymerization of lignin is carried out at 190° C.

15.-36. (canceled)

37. The method of claim 4, wherein producing syringol from the depolymerized lignin composition comprises:
treating the depolymerized lignin composition with an oxidation agent, wherein the oxidation agent converts syringaldehyde to syringic acid; and
performing a decarboxylation reaction to convert syringic acid to syringol, thereby affording a syringol-containing crude product.

38. The method of claim 37, wherein the oxidation agent comprises an oxygen gas, air or oxygen-enriched air, and wherein the oxidation agent further comprises a carrier gas selected from nitrogen, argon and helium.

39. (canceled)

40. The method of claim 37, wherein treating the depolymerized lignin composition with an oxidation agent is carried out in the presence of bis(methoxypropyl) ether.

41. The method of claim 37, wherein performing the decarboxylation reaction comprises contacting the depolymerized lignin composition with a decarboxylation catalyst, wherein the decarboxylation catalyst converts syringic acid to syringol, and wherein the decarboxylation catalyst comprises CuCl, Cu(NO₃)₂, Cu₂O, CuO, Cu, CuSO₄, Cu(OAc)₂ or CuCl₂ and is the decarboxylation catalyst is present in an amount from 0.05 to 2.5 eq. relative to syringic acid.

42-43. (canceled)

44. The method of claim 37, wherein the decarboxylation reaction is carried out at a temperature of 120° C. or more, 150° C. or more, 180° C. or more, 200° C. or more, or 250° C. or more.

45-52. (canceled)

53. The method of claim 1, further comprising pretreating the biomass by a mechanical means.

54. The method of claim 53, wherein pretreating the biomass comprises milling or grinding the biomass to afford biomass particles.

55. The method of claim 54, wherein an average size of the biomass particles is from 70 μm to 1,200 μm.

56. The method of claim 1, wherein:
(A) the biomass comprises nutshells, wherein the nutshells are selected from shells of walnuts, peanuts, pines, almonds and cashews;
(B) the biomass comprises a part of a hardwood, wherein the hardwood is selected from oak, walnut, beech, maple, ash and poplar;
(C) the biomass comprises corn stover, wheat straw, rice stalk, barley straw, sugarcane bagasse, peanut shell or soybean hull; or
(D) the biomass comprises a bioengineered source obtained from microbes.

57-118. (canceled)

119. A reactive diluent comprising syringol methacrylate or syringol acrylate formed from a syringol produced by the method according to claim 1.

120-144. (canceled):
